(19) United States
(12) Patent Application Publication
(10) Pub. No.: US 2005/0079613 A1
(43) Pub. Date: Apr. 14, 2005

(54) Downregulation of cell surface glycoproteins by a family of human ubiquitin ligases

(75) Inventors: Klaus Fruh, Portland, OR (US); Bianca Nerenberg, Beaverton, OR (US); Eric Bartee, Portland, OR (US); Mandana Mansouri, Portland, OR (US); Kristine Gouveia, Portland, OR (US)

Correspondence Address: DAVIS WRIGHT TREMAINE, LLP, 2600 CENTURY SQUARE, 1501 FOURTH AVENUE, SEATTLE, WA, 98101-1688, US

(73) Assignee: Oregon Health & Science University, Portland, OR (US)

(21) Appl. No.: 10/624,727

(22) Filed: Jul. 21, 2003

Related U.S. Application Data
(60) Provisional application No. 60/397,136, filed on Jul. 19, 2002.

(51) Int. Cl. C12N 9/64; C12N 15/85

(52) U.S. Cl. 435/455; 435/226

(57) ABSTRACT

According to the present invention, eight human RING-CH-containing genes are human transmembrane ubiquitin ligase proteins, are homologues of the viral K3-family, and perform functions similar to their viral counterparts. One of these proteins, MARCH-IV, is able to downregulate MHC I and CD4 in a fashion similar to that afforded by the viral immune evasion proteins. This is the first cellular gene product identified that downregulates surface expression of MHC I. The MARCH-family of proteins regulates endocytosis of cell surface receptors (e.g., transferrin receptor, histocompatibility antigens and Fas; type I as well as type II transmembrane domains) via ubiquitination. Particular embodiments provide drug targets for inhibiting the internalization and degredation of various cell surface receptors. Further embodiments provide methods for treating or preventing cancer and other disorders (e.g., leukemia, mental retardation, and L-thalassemia), which methods comprise the administration of a MARCH antagonist or pharmaceutical composition thereof. Screening methods for identification of therapeutic compounds that are modifiers of MARCH activity, are also encompassed by the present invention.

### CROSS-REFERENCE TO RELATED APPLICATIONS

This application claims benefit of priority to U.S. Provisional Patent Application No. 60/397,136, filed 19 Jul. 2002.

### STATEMENT REGARDING FEDERALY FUNDED RESEARCH

This work was supported by grant NIH R01 CA64011. The United States government has certain rights in this invention.

### FIELD OF THE INVENTION

The present application is directed to a family of mammalian transmembrane ubiquitin ligase proteins and their method of use. The application discloses that human homologues of the viral K3-family perform functions similar to their viral counterparts. In particular, the dislclosed MARCH-family of proteins regulates endocytosis of cell surface receptors via ubiquitination.

### BACKGROUND OF THE INVENTION

Ubiquitination plays a central role in diverse cellular functions such as signal transduction, cell cycle progression, differentiation and apoptosis (Hershko & Ciechanover, Annu Rev Biochem 67:425-479, 1998). Many of these functions are the consequence of ubiquitin-mediated proteasomal degradation (Hochstrasser, M., Annu Rev Genet 30:405-439, 1996). Ubiquitination also regulates the sorting of proteins along the endocytic route (Hicke, L., Faseb J 11:1215-1226, 1997); for instance, ubiquitination redirects recycling epidermal growth factor receptor (EGFR) into late endosomes, and ultimately lysosomes (Joazeiro et al., Science 286;309-312, 1999). A key step along this route is the formation of multivesicular bodies (MVBs) that are generated by invagination and intravesicular budding of endosomal membranes. Membranes inside these MVBs are delivered to lysosomes enabling the lysosomal destruction of lipids as well as transmembrane proteins (Lemmon & Traub, Curr Opin Cell Biol 12:457-466, 2000). Ubiquitin-dependent sorting of transmembrane proteins into MVB's is directed by a series of endosomal sorting complexes called ESCRT-I, II and III (Babst et al., Dev Cell 3:271-282, 2002; Babst et al., Dev Cell 3:283-289, 2002; and Katzmann et al., Cell 106:145-155, 2001). Most of the proteins in these complexes belong to a group of 15 genes called the class E vacuolar protein sorting (vps) genes that are conserved from yeast to man (Odorizzi et al., Cell 95:847-858, 1998). In yeast, deletion of any of these genes results in the mislocalization of protein cargo to the vacuole membrane and the formation of large aberrant endosomal structures called class E compartments (Raymond et al., Mol Biol Cell 3:1389-1402, 1992).

Protein targets are selected for ubiquitination by ubiquitin-ligases, also called E3s. E3s perform their function by simultaneously interacting with a substrate as well as one of the ubiquitin conjugating enzymes (E2s), which receive activated ubiquitin from the ubiquitin activating enzyme (E1). The specificity of ubiquitination is thus largely controlled by E3s (Pickart, C. M., Annu Rev Biochem 70:503-533, 2001). For example, the ubiquitination of EGFR is mediated by the E3 c-cbl (Joazeiro et al., Science 286:309-312, 1999). In the absence of c-cbl, EGFR is not downregulated after ligand binding which leads to malignant growth. The two major families of E3s contain either HECT-domains (homologous to E6 AP c-terminus) or RING-domains (really interesting new gene) (Joazeiro & Weissman, Cell 102;549-552, 2000; and Pickart, 2001, supra) Although these domains show little sequence similarity, they share a common binding site on an E2 molecule, as revealed by two co-crystal structures of the E2 enzyme UbCH7 bound to either the RING-domain of c-cbl or the HECT domain of E6AP (Zheng et al., Cell 102:533-539, 2000).

The RING-domain belongs to a large class of zinc finger motifs and is characterized by a conserved series of cysteines and histidines: the RING-finger (C3HC4), the RING-H2-finger (C3H2C3) (Saurin et al., Trends Biochem. Sci. 21:208-214, 1996), the LIM-finger (C2HC5) (Dawid et al., Trends Genet. 14:156-162, 1998) and the TRIAD-finger (C6HC) (van der Reijden et al., Protein Sci. 8:1557-1561, 1999). A conserved sequence structurally related to the RING-finger is the plant homeo domain (PHD) or leukemia-associated protein domain (LAP), a double zinc-finger motif of unknown function widely found in eukaryotic organisms (Aasland et al., Trends Biochem. Sci. 20:56-59, 1995 and Saha et al., Proc. Natl. Acad. Sci. USA 92:9737-9741, 1995) which is characterized by the C4HC3-sequence CX₁₋₂CX₉₋₂₁CX₂₋₄CX₄₋₅HX₂CX₁₂₋₄₆CX₂C with X being any amino-acid. The PHD/LAP-domain is involved in several human disorders with deletions and missense mutations leading to several types of leukemia, mental retardation or α-thalassemia (see references in (Capili et al., EMBO J. 20:165-177, 2001)). The solution structure of the PHD/LAP-fingers of the KAP-1 co-repressor (Capili et al., 2001, supra) and the Williams Beuren transcription factor (Pascual et al., J. Mol. Biol. 304:723-729, 2000) revealed that the PHD/LAP-domain forms a cross-braced structure with two zinc-ions similar to the RING-finger. In contrast, the LIM-domain binds two zinc-ions in a sequential fashion (Capili et al., 2001, supra).

The PHD/LAP motif is closely related to other double zinc-fingers such as the RING-finger (C3HC4), the RING-H2-finger (C3H2C3) (Saurin, A. J. et al, 1996, supra), the TRIAD-finger (C6HC) (van der Reijden, B. A., et al., 1999, supra), and the LIM-finger (C2HC5) (Dawid, I. B. et al., 1998, supra). LIM-domains are known to mediate protein/protein interactions, mostly by interacting with other LIM-domain proteins, in transcriptional co-activators and cytoskeleton-associated proteins (Dawid, I. B. et al., 1998, supra and Joazeiro, C. A., and A. M. Weissman, Cell 102:549-552, 2000). The solution structures of the PHD/LAP-fingers of the KAP-1 co-repressor (Capili et al., supra) and the Williams Beuren transcription factor (Pascual, J. et al., 2000, supra) revealed that the PHD/LAP-domain forms a cross-braced structure with two zinc-ions that is similar to the RING-finger, but different from the LIM-domain which binds two zinc-ions in a sequential fashion. This structural relationship suggests that the PHD/LAP domain is functionally closely related to the RING domain.

A subfamily of the PHD/LAP domain was discovered in gamma-2 herpesviruses and poxvirus as well as several eukaryotic genomes (Nicholas, J. et al., J. Virol. 71:1963-1974, 1997). The name BKS (Bovine herpesvirus 4, KSHV, Swinepox) was suggested for this motif with the consensus C^{W}/_{R}ICX₁₀₋₁₄CXCX₄₋₇HX₂CX₁₀₋₁₉CX₂C. Yeast contains a single protein with this motif, SSM4 or DOA10. SSM4 is responsible for the ER-associated degradation of a subclass of hydrophobic proteins by the proteasome (Swanson, R., et al., Genes Dev. 15:2660-2674, 2001). The BKS-domain of this protein was further shown to act as an ubiquitin ligase in vitro. Given both the sequence and functional similarity to the RING- and RING-H2 domain, it has been proposed to rename the BKS subtype of PHD/LAP motifs as RING-CH (Swanson et al., 2001, supra), a nomenclature adopted here.

Sequence analysis of the Kaposi's sarcoma associated herpesvirus (KSHV) genome revealed two open reading frames, K3 and K5, encoding PHD/LAP-domain proteins. The PHD/LAP-finger in both of these proteins is present near the aminoterminus and is closely followed by two transmembrane domains. This structure and topology is conserved within other gamma-2 herpesvirus genomes as well as in poxvirus genomes (Nicholas, J. et al., 1997, supra). It was further noted that the PHD/LAP-domains found in the viral proteins represented a subclass of the PHD/LAP motif. This subclass was also found in genomes of lower eukaryotic organisms (Nicholas, J. et al., 1997, supra).

The two RING-CH-proteins, K3 and K5, in the genome of Kaposi's sarcoma associated herpesvirus (KSHV), as well as the single K3-gene of the KSHV-related murine gamma-2 herpesvirus (MHV) 68 were determined by several groups to encode proteins that inhibit the surface expression of major histocompatibility class I molecules (MHC I) (Coscoy, L., and D. Ganem, Proc. Natl. Acad. Sci. USA 97:8051-8056, 2000; Ishido, S. et al., Immunity 13:367-374, 2000a; Stevenson, P. G. et al., Proc. Nat'l. Acad. Sci. USA 97:8455-8460, 2000). In addition to MHC I, KSHV-K5 was found to downregulate surface expression of the co-stimulatory molecules ICAM-1 and B7.2 (Ishido S., et al., J. Virol. 74:5300-5309, 2000b and Coscoy, L. et al., J. Cell. Biol. 155:1265-1974, 2001a). Target molecules for KSHV-K3 and KSHV-K5 are rapidly internalized from the cell surface and destroyed in the lysosomes (Coscoy, L., and D. Ganem, Proc. Nat'l. Acad. Sci. USA 97:8051-8056, 2000; Ishido, S. et al., 2000b, supra). In contrast, MHV68-K3 causes MHC-I to be degraded by the proteasome (Boname, J. M. and P. G. Stevenson, Immunity 15:627-636, 2001). Both KSHV and MHV68 proteins require lysines in the cytoplasmic tail of their target molecules for downregulation (Boname and Stevenson, 2001, supra; Coscoy and Ganem, J. Clin. Invest. 107:1599-1606, 2001b), as well as in KSHV-K5 (Coscoy and Ganem, 2001, supra) and KSHV-K3 transfectants. The isolated RING-CH-domain of KSHV-K5 also displays ubiquitin ligase activity in vitro (Coscoy et al., 2001, supra).

Therefore, the gamma-2 herpesvirus RING-CH proteins act as E3 enzymes, which mediate the ubiquitination of the cytosolic tails of target transmembrane proteins (e.g. MHC-1, CAM-1 AND B7.2) (Boname and Stevenson, 2001, supra; Coscoy et al., 2001, supra).

Moreover, the isolated PHD/LAP-domain of KSHV-K5 displays ubiquitin ligase activity in vitro (Coscoy and Ganem, 2001, supra). A homologous gene, M153R, in the poxvirus Myxoma-virus has been shown to downregulate MHC I surface expression (Guerin, J. L. et al., J. Virol. 76:2912-2913, 2002). Unpublished data suggests that M153R acts by a mechanism very similar to KSHV-K5 and KSHV-K3. This is consistent with scheme by which both the gamma-2 herpesvirus and poxvirus PHD/LAP-proteins act to ubiquitinate the cytoplasmic tail of their targets. Since ubiquitin is known to serve as both a lysosomal and proteasomal targeting signal, this modification could then mediate the destruction of the surface receptors.

Sequences homologous to the gamma-2 herpesvirus RING-CH proteins are also present in poxvirus genomes (Nicholas et al., 1997, supra), and the question as to whether these homologues perform a similar function and act by a similar mechanism than the herpesviral proteins has been investigated (Guerin et al., 2002 supra; Mansouri et al., J. Virol. 77:1427-40, 2003). Cells transfected with the M153R gene of myxomavirus (MV) displayed reduced surface expression of MHC I as well as the T cell co-receptor CD4 whereas deletion of M153R from the genome of MV restored surface expression (Guerin et al., 2002, supra; Mansouri et al., 2003, supra). These surface receptors were rapidly internalized by endocytosis and sorted to lysosomes via the MVB pathway that involves ubiquitination of the cytoplasmic tail (Mansouri et al., 2003, supra). Thus, gamma-2 herpesviruses and poxvirus share a family of “immune evasion proteins” that function as ubiquitin ligases. Several names have been suggested for this family, such as modulators of immune recognition (MIR) (Coscoy et al., 2001, supra) or Scrapins (Guerin et al., 2002, supra). This viral protein family is referred to by the present inventors as the K3-family (Früh et al., Virus Research 88:55-69, 2002).

Since poxviruses and herpesviruses are unrelated viral families, it is likely that the RING-CH proteins in their genomes originated in eukaryotic host genomes. Several examples exist for host-related immune evasion genes shared between herpesviruses and poxviruses (McFadden, G. and P. M. Murphy, Curr. Opin. Microbiol. 3:371-378, 2000).

Although viral proteins, such as in Kaposi's sarcoma associated herpesvirus and the the poxvirus Myxoma-virus, have been identified that exhibit ubiquitin-ligase activity, there is a need to identify mammalian proteins with analagous functions. Further, there is a need to identify mammalian proteins with homologous structure to the viral proteins exhibiting ubiquitin-ligase activity.

These and other limitations and problems of the past are solved by the present invention.

### BRIEF SUMMARY OF THE INVENTION

A family of transmembrane ubiquitin ligases proteins and their method of uses is disclosed and described.

According to the present invention, human homologues of the viral K3-family perform functions similar to their viral counterparts. Eight human RING-CH-containing genes, the membrane-associated RING-CH (MARCH) protein family have been identified and are disclosed herein. One of these proteins, MARCH-IV, is able to downregulate MHC I and CD4 in a fashion similar to that afforded by the viral immune evasion proteins. Moreover, in the presence of MARCH-IV, MHC I was internalized via the MVB pathway in a lysine-dependant manner and degraded in lysosomes. This is the first cellular gene product identified that downregulates surface expression of MHC L Based on the results of the experiments described herein, the MARCH-family of proteins regulates endocytosis of cell surface receptors via ubiquitination, consistent with a scenario in which this protein family gave rise to the viral immune evasion proteins.

In one embodiment, a family of proteins that function as ubiquitin ligases and reduce the surface levels of type I as well as type II transmembrane domains is shown and described. In one aspect of this embodiment, member proteins (MARCH proteins) of this family have an amino-terminal ubiquitin ligase domain (PHD/LAP-domain or RING-CH domain) and two or more transmembrane domains.

In another embodiment, a method is shown and described of using one or more members of the family of transmembrane ubiquitin ligases as drug targets for inhibiting the internalization and degredation of various cell surface receptors.

Yet another disclosed embodiment comprises a family of transmembrane ubiquitin ligases having utility to mediate internalization of cell surface receptors. In one aspect of this embodiment, the cell surface receptors include, but are not limited to, transferrin receptor, histocompatibility antigens and Fas.

In a further embodiment, the present invention provides methods for treating cancer, such as leukemia, which methods comprise the administration of a MARCH antagonist or pharmaceutical composition thereof. In a related aspect, the present invention provides methods for preventing the development of cancer, which methods also comprise the administration of a MARCH antagonist or pharmaceutical composition thereof. More specifically, an amount of a MARCH antagonist or pharmaceutical composition thereof is administered to a patient afflicted with or predisposed to a cancer, such as leukemia, whereby the interaction between MARCH and its receptor (MARCH-R) is inhibited.

In another aspect, the present invention provides methods for preventing or treating cancer. These methods comprise administering to a mammal afflicted with a cancer a therapeutically effective amount of a MARCH antagonist thereby preventing the cancer or reducing the severity of the cancer.

By the present invention, MARCH antagonists may be monoclonal or polyclonal antibodies, including humanized or human antibodies, or epitope binding fragments thereof. Alternatively, MARCH antagonists include suitable proteins or peptides or other small molecules that bind MARCH thereby inhibiting the interaction of MARCH and MARCH-R. The compositions of the present invention may further comprise a pharmaceutically acceptable carrier or stabilizer suitable for in vivo administration. In particular embodiments, these compositions may be further combined with additional agents efficacious against cancer.

The present invention thus provides novel compositions and methods that are effective in treating cancer, such as leukemia, mental retardation, and L-thalassemia.

In particular embodiments, the antagonist blocks the interaction between MARCH and its receptor (target), MARCH-R, and in a specific embodiment, the antagonist is or comprises an antibody that binds to MARCH. According to the methods of the invention, the antibody may be a monoclonal antibody (or is or comprises an epitope-binding fragment thereof), and in one embodiment the antibody is a humanized monoclonal antibody (or is or comprises an epitope-binding fragment thereof). In another embodiment, the antibody is a human monoclonal antibody (or is or comprises an epitope-binding fragment thereof).

The invention also provides a method for treating a mammal afflicted with a cancer comprising administering to the mammal a therapeutically effective amount of a MARCH antagonist thereby reducing the severity of the cancer, wherein the mammal is human.

In particular embodiments, the MARCH antagonist is administered intraperitoneally or intradermally.

This family of proteins may be used as novel targets in many different therapeutic areas, including but not limited to cancer, autoimmune diseases, and neurological diseases.

Screening methods for identification of therapeutic compounds that are modifiers of MARCH activity, are also encompassed by the present invention.

Specifically, the present invention provides a method for treating or preventing a condition affected by endocytosis or down-regulation of cell surface receptors, comprising administering to a patient in need thereof a therapeutically effective amount of a MARCH-protein antagonist or pharmaceutical composition thereof, wherein the antagonist inhibits MARCH-protein-mediated endocytosis or down-regulation of type I or type II cell surface receptors. Preferably, the MARCH antagonist is selected from the group consisting of anti-MARCH-protein antibodies or eptitope-binding fragments thereof, MARCH-specific antisense oligomers, proteins and polypeptides, small molecule MARCH inhibitors of MARCH-protein-mediated ubiquitination, and combinations thereof. Preferably, the MARCH-specific antisense oligonucloetide comprises a sequences of at least 12 contiguous nucleotides of a sequence selected from the group consisting of SEQ ID NOS:38, 40, 42, 44, 46, 48, 50 and SEQ ID NO:52, and complements thereof. Preferably, the MARCH-protein is selected from the group consisting of MARCH-I through MARCH-VII, and MARCH-VIII, corresponding to SEQ ID NOS:39 and 54, SEQ ID NOS:41 and 55, SEQ ID NOS:43 and 56, SEQ ID NOS:45 and 57, SEQ ID NOS:47 and 58, SEQ ID NOS:49 and 59, SEQ ID NOS:51 and 60, SEQ ID NOS:53 and 61, respectively. Preferably, the MARCH-protein-mediated endocytosis of type I or type II cell surface receptors, involves a receptor selected from the group consisting of transferrin receptor, FAS (APO-1/CD95), MHC-I and other histocompatibility antigens, HLA-A2.1 or CD4, ICAM-1, B7.2, and combinations thereof. Preferably, the condition is selected from the group consisting of leukemia and other cancers, mental retardation, thalessemia, autoimmune disease and neurological disease.

Additional embodiments provide methods for identifying test compounds having therapeutic activity for a condition affected by endocytosis of cell surface receptors, comprising: contacting a test compound with a MARCH-protein target/receptor and a functional MARCH-protein wherein at least one of the proteins bears a detectable label; assaying any resulting MARCH-protein target/receptor:MARCH-protein complex for the presence of the label; and determining whether the test compound antagonizes binding of the MARCH-protein target/receptor to the MARCH-protein, whereby test compounds that antagonize said binding are, at least in part, identified as therapeutic compounds. Preferably, the MARCH-protein is selected from the group consisting of MARCH-I through MARCH-VII, and MARCH-VIII, corresponding to SEQ ID NOS:39 and 54, SEQ ID NOS:41 and 55, SEQ ID NOS:43 and 56, SEQ ID NOS:45 and 57, SEQ ID NOS:47 and 58, SEQ ID NOS:49 and 59, SEQ ID NOS:51 and 60, SEQ ID NOS:53 and 61, respectively. Preferably, the MARCH-protein target/receptor is selected from the group consisting of transferrin receptor, FAS (APO-1/CD95), MHC-I and other histocompatibility antigens, HLA-A2.1 or CD4, ICAM-1, B7.2, and combinations thereof. Preferably, either the functional MARCH-protein target/receptor or the functional MARCH-protein is immobilized onto a solid phase. Preferably, the MARCH-protein target/receptor or the MARCH-protein is labeled with a radiolabel, a fluorescent reporter or quencher moiety, an enzymic label that catalyzes a colorometric or fluorometric change, or combinations thereof.

Further embodiments provide methods for identifying test compounds having therapeutic activity for a condition affected by endocytosis of cell surface receptors, comprising: contacting a test compound with a cell expressing a functional MARCH-protein target/receptor and a MARCH-protein; and determining whether the test compound antagonizes at least one of MARCH-protein-mediated ubiquination activity, or MARCH-protein-mediated receptor endocytosis, whereby test compounds that antagonize at least one of such activity are identified as therapeutic compounds. Preferably, the cell expresses a recombinant MARCH-protein target/receptor or a recombinant MARCH-protein. Preferably, determination of antagonism of MARCH-protein-mediated activity is based on an assay selected from the group consisting of: inhibition of MARCH-protein-mediated endocytosis assays, receptor up- or down-regulation assays, cell motility assays, cell growth rate assays, apoptosis assays, ubiquination assays, and MARCH-protein-mediated or MARCH-protein target/receptor-mediated signal transduction assays. Preferably, the MARCH-protein-mediated endocytosis assay is based on measurement of a cell-surface receptor selected from the group consisting of transferrin receptor, FAS (APO-1/CD95), MHC-I and other histocompatibility antigens, HLA-A2.1 or CD4, ICAM-1, B7.2, and combinations thereof. Preferably, the MARCH-protein-mediated or MARCH-protein target/receptor-mediated signal transduction assay is based on measurement of cellular processes selected from the group consisting of phosphorylation or activation of an intracellular protein, organization of the actin cytoskeleton, gene transcription, lipid metabolism, vesicle trafficking, cellular transformation, cellular death, and combinations thereof, whereby test compounds that alter said cellular process, relative to those of control cells, are identified as therapeutic compounds. Preferably, the condition characterized by MARCH-protein-mediated cell growth, cell motility, or inhibition of endocytosis is cancer (e.g., leukemia), mental retardation or thalessemia.

Yet further embodiments provide pharmaceutical compositions, comprising an antagonist of a MARCH-protein, and a pharmaceutically acceptable carrier or diluant. Preferably, the MARCH-protein antagonist is a MARCH-specific antisense oligonucleotide comprising at least 12 contiguous nucleotides of a sequence selected from the group consisting of of SEQ ID NOS:38-45, and complements thereof.

Still further embodiments provide anti-MARCH-protein antibodys. Preferably, the antibody is a monoclonal antibody. Preferably, the monoclonal antibody is a single-chain antibody, chimeric antibody, humanized antibody or Fab fragment.

Additional embodiments provide methods for inhibiting endocytosis or down-regulation of cell-suface receptors, comprising contacting a cell, having a cell-surface receptor that is at least to some extent subject to MARCH-protein-meidated endocytosis, with a MARCH-protein antagonist, whereby said endocytosis or down-regulation is, at least to some extent, inhibited. Preferably, the MARCH antagonist is selected from the group consisting of anti-MARCH-protein antibodies or eptitope-binding fragments thereof, MARCH-specific antisense oligomers, proteins and polypeptides, small molecule MARCH inhibitors of MARCH-protein-mediated ubiquitination, and combinations thereof. Preferably, the MARCH-specific antisense oligonucloetide comprises a sequences of at least 12 contiguous nucleotides of a sequence selected from the group consisting of SEQ ID NOS:38, 40, 42, 44, 46, 48, 50 and SEQ ID NO:52, and complements thereof. Preferably, the MARCH-protein is selected from the group consisting of MARCH-I through MARCH-VII, and MARCH-VIII, corresponding to SEQ ID NOS:39 and 54, SEQ ID NOS:41 and 55, SEQ ID NOS:43 and 56, SEQ ID NOS:45 and 57, SEQ ID NOS:47 and 58, SEQ ID NOS:49 and 59, SEQ ID NOS:51 and 60, SEQ ID NOS:53 and 61, respectively. Preferably, the MARCH-protein-mediated endocytosis or down-regulation of type I or type II cell surface receptors, involves a receptor selected from the group consisting of transferrin receptor, FAS (APO-1/CD95), MHC-I and other histocompatibility antigens, HLA-A2.1 or CD4, ICAM-1, B7.2, and combinations thereof.

The invention will best be understood by reference to the following detailed description of the preferred embodiment, taken in conjunction with the accompanying drawings. The discussion below is descriptive, illustrative and exemplary and is not to be taken as limiting the scope defined by any appended claims.

### BRIEF DESCRIPTION OF THE DRAWINGS

The patent or application file contains at least one drawing executed in color. Copies of this patent or application publication with color drawing(s) will be provided by the Office upon request and payment of the necessary fee.

FIGS. 1a, 1b and 1c show that the K3-family of proteins is conserved among viruses and a broad range of eukaryotic organisms, including human. Specifically, FIGS. 1a, 1b and 1c show an alignment of various PHD/LAP finger-containing proteins, including that of gamma2-herpesvirus and poxvirus, as well as several eukaryotes.

FIGS. 1d and 1e show that the MARCH family of proteins is characterized by an N-terminal PHD/LAP-finger and several predicted transmembrane domains. Specifically, an alignment is shown of the PHD/LAP finger from the eight human MARCH proteins (1-VIII) and the known viral K-3-family proteins. The totally conserved C₄HC₃ motif present in all PHD/LAP fingers is shown boxed in dark grey, while several other highly conserved amino acids are shown boxed in light grey. The hydoropathy plots (FIG. 1e) for each MARCH protein are analyzed using TMPRED (ch.embnet.org), and the number of transmembrane domains predicted is shown to the left of the PHD/LAP alignment. The location of the PHD/LAP-finger in each protein is shown by a black box under the hydorphathy plot (FIG. 1e). All human MARCH proteins as well as all viral K3-family proteins are predicted to have an even number of transmembrane segments, likely placing both their N and C-termini on the same side of the membrane. All PHD/LAP-fingers are located at the N-terminal end of the proteins prior to any predicted transmembrance domain. This is particularly striking in MARCH-VI, which contains 12 predicted transmembrane segments and MARCH-VII, which is predicted to have 500 amino-acids prior to the PHD/LAP-finger. Pubmed accession numbers are given for all proteins as well as Unigene numbers for all human MARCH genes.

FIG. 2 shows that MARCH-I and MARCH-II have different tissue distributions. Real-time PCR was used to quantitate mRNA levels of MARCH-I (white bars) and MARCH-II (black bars) in various tissues. A known amount of plasmid containing either the MARCH-I or MARCH-II open reading frame (ORF) was used to calculate copy number. MARCH-I was expressed predominantly in the brain while MARCH-II was ubiquitinously expressed.

Fiure 3 shows that MARCH-I and MARCH-II both localize primarily to the lysosomal compartment. HeLa cells were transfected with Flag tagged MARCH-I (A) or MARCH-II (B). 20 hours post-transfection, cells were stained with antibodies against various cellular markers (EEA-1 (early endosomes), Lamp-i (lysosomes), calnexin (ER), golgin-97 (Golgi)) using an Alexa Fluor:568 (Molecular Probes) secondary antibody (red). MARCH-I and MARCH-II were stained with an α-Flag:FitC (Sigma) conjugated antibody (green). Colocalization is visualized as yellow in the merged panel.

FIG. 4 shows down-regulation of surface receptors by MARCH-I, MARCH-II, and MARCH-VIII. HeLa cells were transfected with GFP and either pUHD10-1 (black), or MARCH-I, MARCH-II, MARCHVIII (white). At 24 hours post-transfection, cells were trypsinized and stained with monoclonal antibodies against either transferrin receptor, Fas, or MHC-I using a PE conjugated secondary. Cells were then analyzed via flow cytometry. The plots shown are gated on GFP-expressing live cells. MARCH-I exhibited a strong downregulation of TFr, but had no effect on Fas: or MHC-I (not shown). MARCH-II down regulated TFr very slightly and did not affect MHC-I (not shown), but had a strong effect on Fas. MARCH-VIII down regulated both TFr and MHC-I but did not affect Fas.

FIG. 5 shows that both MARCH-I and MARCH-II form high molecular weight complexes. MARCH-I and MARCH-II were transfected into HeLa cells, metabolically labeled, and immunoprecipitated with anti-Flag anitbody. Bands corresponding to the molecular weight of monomeric MARCH-I (30 kDa) and MARCH-II (26 kDa) (dark arrows) are clearly visible in the initial immunoprecipitation (left panel A) as well as several additional bands of higher molecular weight. Several of these additional bands (white arrows) were confirmed to contain MARCH-I or MARCH-II by western blotting the initial immunoprecipitation with anti-Flag antibody (right panel B). The heavy and light chains of the anti-Flag antibody used in the immunoprecipitation are also shown in the western blot.

FIG. 6 shows that purified PHD domains of both MARCH-I and MARCH-II function as E3 ubiquitin ligases in vitro. Purified MARCH-I and MARCH-II PHD domains expressed as GST-fusion proteins were incubated at 30° C. in the presence of ubiquitin, ATP, E1 and various E2's. Samples were boiled in SDS sample buffer and examined via western blotting with an anti-ubiquitin antibody. The appearance of a high molecular weight species upon the addition of purified PHD domain was interpreted as positive E3 activity. The purified RING domain of HSV1-ICPO and reaction buffer lacking either PHD domain are included as positive and negative controls, respectively.

ures 7A, 7B and 7C show the alignment and tissue distribution of Human homologues of viral K3 family proteins. FIG. 7A shows a sequence comparison of human K3 family homologues. The RING-CH domain from the eight identified human MARCH proteins (I-VIII) and the known viral K3-family proteins were aligned using Vector NTI 7.0 software. The C₄HC₃ motif characterizing the RING-CH-domain is shown boxed in dark gray, while other conserved amino acids are shown boxed in light gray. The hydropathy plots (FIG. 7B) for each MARCH protein were generated using TMPRED (www.ch.embnet.org). The location of the RING-CH domain is shown by a black box under the hydropathy plot, while the number of transmembrane domains predicted is shown to the right of the RING-CH alignment. DNA accession numbers and Unigene numbers are given for the MARCH proteins, while protein accession numbers are given for viral proteins.

FIG. 7C shows expression of MARCH-I, II, IV and VIII transcripts as measured by real-time PCR in a panel of human tissue cDNAs (Clonetech), or in HeLa cell-derived cDNA. Expression of each gene was quantified using absolute standards created from plasmid DNA. Concentrations of cDNAs were normalized using the housekeeping genes GAPDH and β-actin. There are several protein predictions for MARCH-IV. Unigene predicts a truncated protein of 132 amino acids due to the exclusion of an exon found in several cloned cDNA's. The hydropathy plot shown is for the longest predicted open reading frame of KIAA1399 which contains an amino-terminal hydrophobic domain. However, multiple forms of MARCH-IV are expressed (see FIG. 11).

FIG. 8 shows the localization of MARCH proteins to intracellular membranous organelles. HeLa cells were transfected with C-terminally Flag-tagged versions of each MARCH protein. 20 hours post-transfection, cells were stained with antibodies against various cellular markers (top panel in red): (EEA-1 (early endosomes), Lamp-1 (lysosomes), calnexin (ER), golgin-97 (Golgi)) using an Alexa Fluor:568 (Molecular Probes) secondary antibody. MARCH proteins (bottom panel in green) were stained with an α-Flag:FitC (Sigma) conjugated antibody. Colocalization is visualized as yellow in the merged panel (middle).

FIG. 9 shows that MARCH proteins act as ubiquitin ligases in vitro. Purified MARCH-I, MARCH-II (top panel), MARCH-IV or MARCH-VIII (bottom panel) RING-CH domains, expressed as GST-fusions, were incubated at 30° C. in the presence of ubiquitin, ATP, E1 and various E2's. The addition of reaction buffer lacking any RING-CH domain (none) is included as a negative control for each E2. Samples were boiled in SDS sample buffer and examined via immunoblotting with an anti-ubiquitin antibody. Ubiquitin ligase activity is indicated by the appearance of a high molecular weight ubiquitinated species not found in the control lanes.

FIGS. 10A through 10E show downregulation of cell-surface glycoproteins by the MARCH-family. FIG. 10A shows HeLa cells that were transfected with 100 ng of plasmid coding for each MARCH protein, as well as GFP, to track transfected cells. Twenty-four hours post-transfection, cells were harvested and surface expression of various surface proteins was measured via flow-cytometry using a PE conjugated secondary. Black arrows label experiments where a surface protein was down-regulated.

FIGS. 10B and 10C show confirmation of expression of all MARCH proteins by immunoprecipitation (FIG. 10B), or by measuring steady-state levels by immunoblot (Figure 10C). For the data of FIGS. 10B and 10C, 200 ng of each MARCH-cDNA was transfected, except for MARCH-I and MARCH-III, where 1000 ng was used.

FIG. 10D shows HeLa cells that were transfected with either MARCH-IV, MARCH-IV full length (which contains an N-terminal extension of 63 amino acids), or MARCH-IV RING-CH mutant (in which two conserved cysteines in the RING-CH domain were mutated to serines). Twenty-four hours post-transfection, the effect of each construct on MHC I was measured via flow cytometry.

FIG. 10E shows HeLa cells that were transfected with MARCH-IV or full length MARCH-IV. Twenty-four hours post-transfection, cells were lysed and MARCH-IV was immune precipitated. Samples were separated and examined via immunoblotting with anti-FLAG antibody.

FIGS. 11A and 11B show that expression levels of MARCH-IV and KSHV-K5 were required for MHC I downregulation. FIG. 11A shows a comparison between the efficiency of MARCH-IV downregulation of MHC-I, to that afforded by KSHV-K5, using the tetracycline-responsive system. HeLa cells were transfected in the presence of 1 μg/ml tetracycline with constitutively expressed A2.1-331/pUHD10-1, and either MARCH-IV/pUHG10-3 or K5/pBI, both under tet-regulation (Paulson, E., Tran, C., Collins, C., and Früh, K., Virology 288:369-378, 2001). GFP was used to track transfected cells. Eight hours post-transfection, the media was removed and replaced with media containing the given amount of tetracycline. Thirty-six hours later the cells were harvested and A2.1 surface expression was measured by flow cytometry using antibody BB7.2 and a PE conjugated secondary antibody. The graph depicts the average percent of GFP+ cells that exhibited A2.1 downregulation. Error bars depict the range of the results from two separate experiments.

FIG. 11B shows the expression of each protein as measured by immunoblotting whole cell lysates with an anti-Flag antibody conjugated to alkaline phosphatase, and quantified using a Typhoon 8600 variable mode imager. The graph depicts protein expression as relative units at each tetracycline concentration.

FIGS. 12A through 12C show endocytosis and lysosomal degradation of MHC I by MARCH-IV. FIG. 12A shows HeLa cells that were transfected with either MARCH-IV/pUHD10-1 (bottom) or empty vector (top), to determine if MARCH-IV functions via enhancing endocytosis. Twenty-four hours post-transfection, the cells were transferred to 4° C. and incubated with the anti-MHC I antiserum K455. Cells were then either fixed immediately (left column) or transferred to 37° C. for 120 minutes (right column). The location of the antibody bound to MHC I was visualized with an Alexaflur:594 secondary antibody.

FIG. 12B shows HeLa cells that were transfected with either A2.1 or A2.1 and MARCH IV constructs, to determine if MARCH-IV substrates are targeted to the lysosomes. Cells were labeled for 30 minutes and chased up to 8 hours in the presence or absence of the indicated inhibitors. HLA-A2.1 molecules were immunoprecipitated using BB7.2 mAb and treated with Endo H.

FIG. 12C shows flow cytometry of MHC I on MARCH-IV-transfected HeLa cells in the presence or absence of concanamycin A. MARCH-IV-mediated MHC I endocytosis and degradation is reduced in the presence of concanamycin A.

FIGS. 13A through 13C show that MARCH-IV and MARCH-VIII require lysines in the ‘tail’ of their targets for endocytosis via the multivesicular body pathway. FIG. 13A shows HeLa cells that were co-transfected with one of a series of A2.1 constructs and either MARCH-IV (middle) or MARCH-VIII (right). Transfectants were identified by GFP cotransfection. Twenty-four hours post-transfection, the cells were harvested and A2.1 surface expression was assayed with flow cytometry using the A2.1 specific antibody BB7.2 and a PE conjugated secondary. A schematic representation of each A2.1 construct is shown on the left. The white bar represents the cytoplasmic tail of A2.1 while the gray bar represents the C-terminal HA tag. Details of the constructs were described previously (Mansouri et al., 2003, supra; Paulson et al., 2001, supra). The location of lysines in each construct is noted with K's.

FIG. 13B shows that removal of lysines from CD4 by exchanging lysines in the cytoplasmic tail also resulted in resistance to MARCH-IV downregulation. Details of the CD4 construct were published previously (Mansouri et al., 2003, supra; Schubert et al., 1998, supra).

FIG. 13C shows HeLa cells that were transfected with wildtype vps4 or a dominant-negative version of vps4 in the presence (right column) or absence (left column) of MARCH-IV. GFP was used to identify transfected cells. Twenty-four hours post-transfection, cells were harvested and the surface expression of MHC I was measured via flow cytometry using a PE conjugated secondary antibody. Co-transfection of vps4 wt and vps4 DN with MARCH-IV partially restored MHC I levels.

### DETAILED DESCRIPTION OF THE BEST MODE

To escape the cellular immune response, viruses have developed immune escape mechanisms that are usually highly host adapted and virus-specific. However, a conserved family of viral proteins is shared by gamma-2 herpesviruses and poxviruses to down-regulate transmembrane glycoproteins involved in immune recognition. Kaposi's sarcoma associated virus (KSHV) K3 and K5 downregulate major histocompatibility complex class I (MHC I) and display amino-terminal PHD/LAP-zinc finger domains followed by two transmembrane domains. This structure is conserved in several gamma-2 herpesvirus and poxvirus proteins. Moreover, the homologous protein M153R of Myxomavirus also downregulates MHC I. A comparison of the molecular mechanism of MHC I downregulation by K5 and M153R revealed differences in subcellular location and substrate specificity, but suggested that both proteins act as ubiquitin ligases adding ubiquitin to the tail of their substrate molecules with ubiquitin acting as an internalisation signal. The finding that two such unrelated viral families share homologous proteins performing similar functions suggests that these proteins were either transferred laterally between different viral species or they were independently acquired from host genomes.

In support of the latter explanation, several related genes in the human genome were herein identified. This viral family represents homologs of cellular membrane bound ubiquitin ligases.

MARCH-I and MARCH-II are mammalian members of the previously described family of viral PHD/LAP domain proteins. Shared characteristics of this family are their amino-terminal PHD/LAP, or RING-CH domain, an even numbers of transmembrane segments, and the formation of high molecular weight complexes. In viruses, this family of ubiquitin ligases is thought to mediate the ubiquitination of the cytoplasmic tail of their target glycoproteins. Therefore, the cellular homologs are likely involved in the degradation of transmembrane proteins (such as MHC-I, ICAM-1 and B7.2). Further support for a role of this family in protein degradation comes from recent observations in yeast. Hochstrasser and colleagues observed that mutants in the Doa10/Ssm4 protein stabilized the shortlived transcription factor Mat2α as well as the ER-resident transmembrane ubiquitin conjugating enzyme ubc6 (Swanson, R. et al., Genes Dev. 15:2660-2674, 2001). Doa10 is related in sequence and predicted transmembrane topology to MARCH-VI, a human protein also called TEB-4 (Swanson, R. et al., 2001, supra). In contrast to the other MARCH-family members, MARCH-VI/TEB-4 encodes 12 predicted transmembrane domains. Whereas Doa10, and presumably MARCH-VI, locate to the ER, both MARCH-I and MARCH-II are localized to post-ER compartments. It could therefore be speculated that a Doa10-like molecule was the original precursor of this protein family, which then diversified for specific functions with respect to target proteins and subcellular location. Swanson et al. proposed the name RING-CH for the PHD/LAP-domain found in the Doa10 protein. Since RING-CH more accurately reflects the function of this domain as ubiquitin-ligase module and thus its functional relationship to the RING domain, it has been adopted herein.

The two-transmembrane domain proteins MARCH-I and MARCH-II are localized to vesicular compartments. These proteins are capable of functioning similarly to the viral proteins KSHV-K3 and KSHV-K5. Recent observations suggest that MHC-I molecules are destined for late endosomal multivesicular bodies prior to their final, lysosomal destination. Ubiquitin is one of the sorting signals for multivesicular bodies, and proteins with ubiquitin-recognition motifs, such as TSG101, play a crucial role in this sorting process. Since both MARCH-I and MARCH-II locate to late endosomal vesicles, they are capable of playing a role in sorting proteins to multivesicular bodies. By contrast, MARCH-VI is capable of being involved in the degradation of ER-resident proteins via the proteasome as observed for DOA10. Two ER-resident ubiquitin-conjugating enzymes, ubc6 and ubc7, were shown to be involved in this process. RING-CH domains of MARCH-I and MARCH-II were examined for promotion of ubiquitination with the murine homologs of yeast ubc6 and ubc7. Whereas MARCH-II was able to function with both proteins, MARCH-I was only active with MmUbc6. By contrast, MARCH-I was more active with UbCH5a compared to MARCH-II. Thus it seems that these proteins are able to function with various E2s, including ER-membrane bound Ubcs. However, different MARCH-proteins differ in their E2-specificity. Given the subcellular transmembrane location of MARCH-I and II, it is likely that additional, endosome-bound E2s will be identified that are the dominant ubiquitin-providers for MARCH-I and II.

The presence of MARCHs in mammalian genomes, as well as related proteins in lower eukaroyotic organisms, suggests that the viral proteins were derived from their cellular homologs. Herpesviridae and Poxyiridae are entirely unrelated viral families with very different life cycles. Although both groups are large DNA viruses it can be assumed that they were separate families even before mammalian organisms even existed. Therefore, it is likely that each viral family independently obtained MARCHs from host genomes. There is ample precedence for host-derived genes in both herpesviral and poxyiral genomes. Moreover, many of the host-derived genes are involved in evading the innate or adaptive immune response of the host. The viral versions of host-derived proteins, however, often assume a new or modified function, or even act as inhibitors of their host counterparts. The MARCHs are capable of the same with cellular targets, such as but not limited to MHC-I, transferrin receptor, CD95, ICAM-1 and B7.2. Logically, it follows that these genes, the MARCH family, are involved in evading the innate or adaptive immune reponse.

The demonstration that MARCH-I, II and VII reduce the surface levels of both type I (MHC-I and CD95) and type II (transferrin receptor) glycoproteins shows that these transmembrance ubiquitin ligases are capable of operating in a fashion very similar to their viral counterparts. The MARCH proteins are therefore capable of being involved in regulating the turnover of surface glycoproteins. Importantly, each of these proteins seems to have a preference for certain substrates, similar to the viral proteins. In combination with the differential tissue distribution, each protein is likely responsible for the downregulation of certain types of receptors in certain tissues.

The observed downregulation of cell surface receptors suggests that MARCH-II and VII, and presumably other MARCH proteins as well, can be used to target specific classes of membrane proteins and regulate their cell surface expression. This could be achieved, for instance, by genetherapy approaches. On the other hand, it is conceivable that inhibiting the function of MARCH proteins might stabilize the surface levels of certain proteins by slowing down their turnover. Therefore, members of the MARCH family are potential targets to inhibit or slow down the turnover for cell surface proteins, such as the transferrin receptor.

Identification of a Family of March Proteins that are Homologous to Viral Immune Evasion Proteins, and that Function as Ubiquitin Liigases

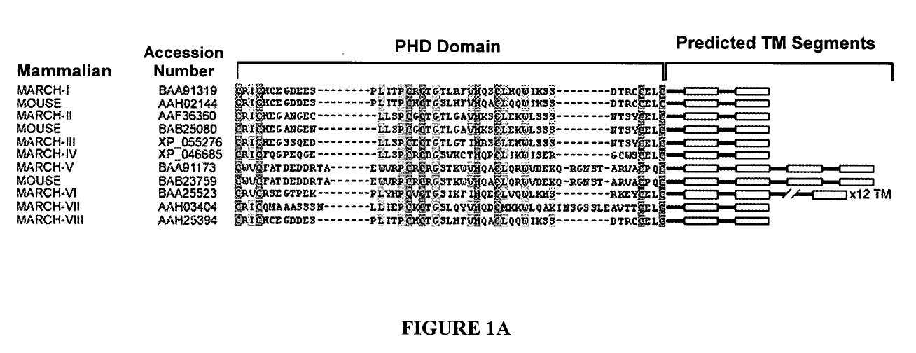

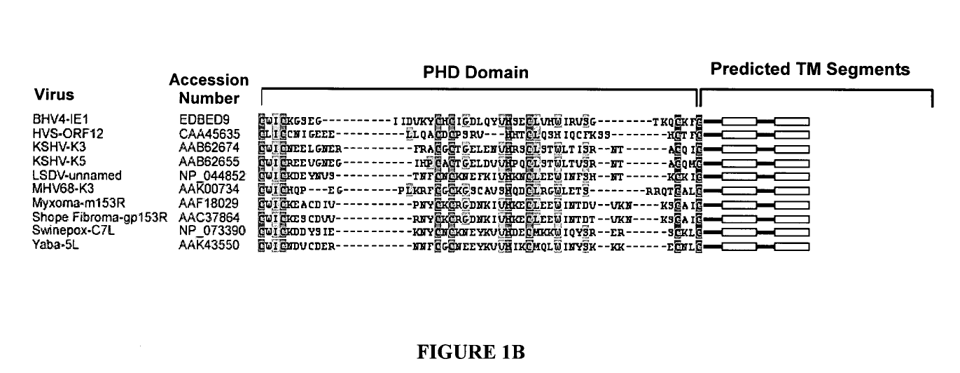

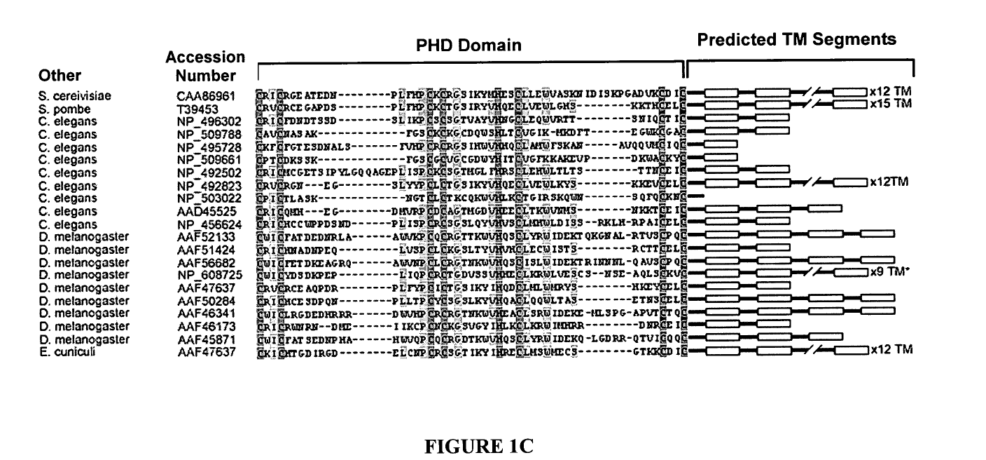

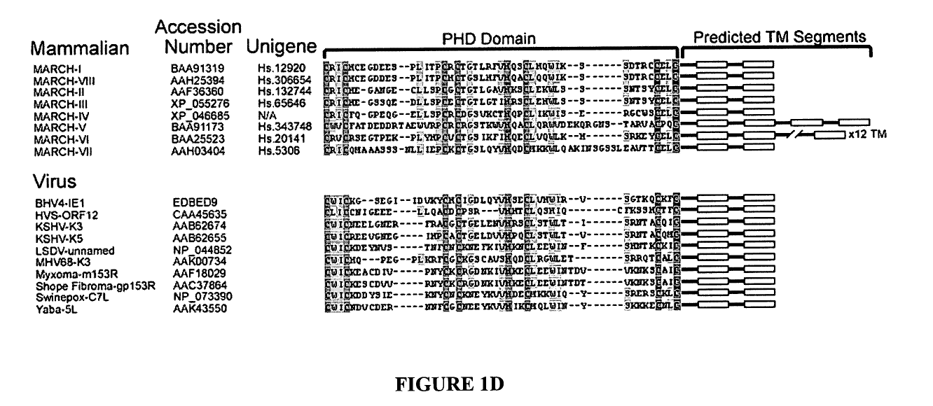

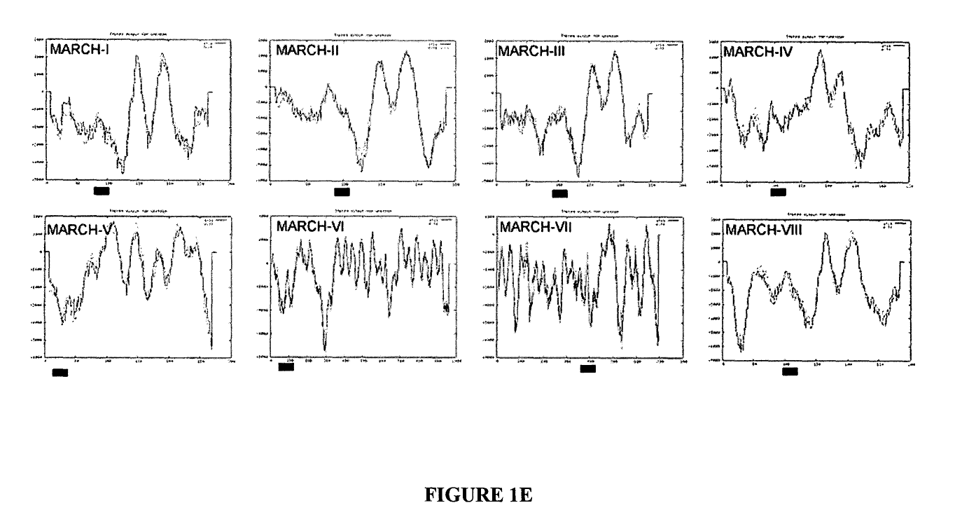

To examine if proteins related to the viral K3-family are also present in mammalian genomes, the Genebank database was searched with the PHD/LAP domains of the viral proteins. BLAST-searches revealed eight hypothetical proteins of unknown function in the human genome that display the viral subclass of PHD/LAP domains (FIGS. 1a through 1e) (TABLE 1, below). For reasons discussed below, this subtype of PHD/LAP domains is referred to herein as the RING-CH domain. Similar to the viral proteins, the human RING-CH domains were all localized at the amino-termini of the predicted proteins. Although other parts of the homologous proteins were not conserved, it is important to note that the RING-CH domains are followed by a central hydrophobic region in all mammalian homologs. This central hydrophobic domain is predicted to represent two, four or 12 transmembrane segments (FIGS. 1a through 1e). All the viral PHD/LAP-finger proteins are predicted to contain two transmembrane domains. Moreover, a topology whereby the membrane of subcellular compartment is traversed twice has been experimentally confirmed. Based on the finding that the RING-CH domain is always followed by predicted transmembrane domains, this mammalian gene family was named herein as Membrane Associated RING-CH Proteins, or MARCH.

The existence of genes homologous to KSHV-K3 and K5 in the genomes of lower eukaryotes was first noted by Nicholas et al., upon completion of the sequence for the corresponding region of the KSHV genome (Nicholas et al., 1997, supra). More recently, several investigators described K3-related genes in mammalian genomes that correspond to the MARCH proteins characterized herein (Holzerlandt et al., Genome Res. 12:1739-1748, 2002; Jenner, R. G. and C. Boshoff, Biochim. Biophys. Acta. 1602:1-22, 2002). The overall sequence homology of these predicted proteins is limited mostly to the RING-CH domain (see FIGS. 7A and 7B).

Using a panel of potential substrates selected as known substrates of K3-family proteins, the present invention provides an efficient and specific downregulation of various cell surface glycoproteins by the cellular homologues. The fact that such a small collection of targets was sufficient to observe downregulation by several of the MARCH-proteins was surprising. The MARCH-proteins that exhibited downregulation showed a RING-CH domain followed by two transmembrane domains typical of the viral proteins. This includes MARCH-IV, which seems to occur in several distinct forms. The partially overlapping, partially distinct substrate specificity is also typical of the viral proteins, e.g., all viral proteins downregulate MHC I, but only KSHV-K5 and MV-M153R downregulate B7.2 and CD4, respectively (Coscoy and Ganem, 2001, supra; Ishido et al., 2000a, supra; Mansouri et al., 2003, supra). The molecular reason for this specificity is not known, but substrates seem to interact transiently with the viral proteins (Hewitt et al., EMBO J. 21:2418-2429, 2002) and substrate recognition seems to reside in the transmembrane regions of the targets (Coscoy et al., 2001, supra).

All MARCH mRNAs are present in HeLa cells at low levels, however, it is not clear whether these low levels play a role in turnover of MHC I or TfR. Nevertheless, the discovery of glycoproteins that are downregulated by the MARCH-family facilitated a comparison of the molecular mechanism of this mammalian protein family with the known K3-family proteins.

The essential, sequence-independent role of lysines in the cytoplasmic tails of substrates strongly suggests that ubiquitination is required for downregulation by the MARCH-family. A central role for ubiquitin is further corroborated by the observed ubiquitin-ligase activity of the RING-CH domain in vitro and by the involvement of the ubiquitin-dependent MVB pathway in removal of the substrates from the cell surface. Very similar observations were reported for the viral proteins. Both herpesviral and poxyiral K3-family proteins were shown to require at least one lysine in the tail (Boname and Stevenson, 2001, supra; Coscoy et al., 2001, supra; Mansouri et al., 2003, supra) and substrate internalization was inhibited by interference with the MVB pathway (Hewitt et al., 2002, supra; Mansouri et al., 2003, supra). For both KSHV-K5 and MV-M153R, it was also shown that the isolated RING-CH domain acts as ubiquitin ligase (Coscoy et al., 2001, supra; Mansouri et al., 2003, supra). The current model for the function of the viral proteins is that they transiently interact with their substrates and mediate the ubiquitination of the cytosolic tail. Our experiments are consistent with the cellular homologues acting in a manner very similar to the one proposed for their viral relatives.

The MARCH-family represents a novel family of ubiquitin ligase with a non-canonical RING-domain for which we adopted the name RING-CH-domain as previously proposed for the SSM4/DOA10 protein (Swanson et al., 2001, supra). This domain expands the RING-E3 family, already the most abundant E3 family with potentially several hundred members (Joazeiro and Weissman, 2000, supra). Each of these E3s is thought to target specific substrates or classes of substrates for ubiquitination (Pickart, C. M., Annu. Rev. Biochem. 70:503-533, 2001). The consequences of ubiquitination mediated by RING-E3s are manifold and range from rapid proteasomal degradation to internalization. Different from the MARCH-family, however, most RING-E3s are either cytosolic, nuclear or peripheral transmembrane proteins (Joazeiro and Weissman, 2000, supra). Only three transmembrane RING-E3s have been described. Der3/Hrd1 (Bordallo et al., Mol. Biol. Cell 9:209-222, 2000), p78 (Fang et al., Proc. Nat'l. Acad. Sci. USA 98:14422-14427, 2001) and SSM4/DOA10 (Swanson et al., 2001, supra). These E3s have all been shown to be involved in the degradation of misfolded proteins in the ER or ER-associated protein degradation (ERAD). During ERAD, misfolded proteins are reverse translocated back into the cytosol for degradation by the proteasome (Plemper, R. K. and D. H. Wolf, Trends. Biochem. Sci. 24:266-270, 1999). MARCH-VI, the human homologue of DOA10/SSM4, is therefore involved in ERAD. In contrast, MARCH-IV directs the internalization of its substrates, a process that ultimately results in lysosomal targeting via MVBs. It seems likely that endocytosis rather than proteasomal degradation will also be the mechanism of the other two-transmembrane MARCH-proteins since the majority seems to locate to the endosomal/lysosomal compartment. Since the only MARCH-related protein found in yeast is DOA10/SSM4 it could be speculated that all MARCH-genes were derived from a DOA10/SSM4-like ancestor. The truncation of the multiple transmembrane segments found in DOA10/SSM4 might have resulted in proteins that localize to compartments other than the ER and ubiquitinate transmembrane proteins at the intersection of exocytosis and endocytosis.

Interestingly, the predicted number of transmembrane domains subsequent to the RING-CH domain is always even. This would be consistent with the type III topology that was experimentally verified for KSHV-K5 and MHV68-K3 (Boname and Stevenson, 2001, supra; Sanchez, D. J., L. Coscoy and D. Ganem, J. Biol. Chem. 18:18, 2001). A cytosolic orientation of the RING-CH domain is also consistent with the cytosolic location of the ubiquitination system.

The membrane association of MARCH-IV implies that the ubiquitin conjugating enzyme is membrane associated or needs to be recruited to the membrane. Indeed, experimental evidence suggests that SSM4/DOA10, p78 and Der3/Hrd1 cooperate with ubc6 and ubc7 (Bordallo et al., 1998, supra; Fang et al., 2001, supra; Gardner, R. G., G. M. Swarbrick, N. W. Bays, S. R. Cronin, S. Wilhovsky, L. Seelig, C. Kim and R. Y. Hampton, J. Cell. Biol. 151:69-82, 2000; Swanson et al., 2001, supra). Both E2s are bound to the ER-membrane via either a transmembrane domain or by interacting with another protein (Biederer, T., C. Volkwein and T. Sommer, Science 278:1806-1809, 1997; Sommer, T. and S. Jentsch, Nature 365:176-179, 1993). It is thus likely that MARCH-VI will interact with the human homologs of ubc6 or ubc7. MARCH-proteins that leave the ER and regulate internalization, however, can be assumed to interact with different E2s. These E2's would need to be accessible at the membrane compartments containing the MARCH-proteins.

In addition to ERAD, MARCH proteins regulate the degradation of misfolded proteins at the cell surface. In the case of MHC I for instance, dissociation of the light chain P2 microglobulin, presumably as a result of dissociation of peptide, results in free heavy chain at the cell surface which undergoes rapid endocytosis and lysosomal degradation (Machold, R. P. and H. L. Ploegh, J. Exp. Med 184:2251-2259, 1996). MARCH-IV plays a role in this process. Ubiquitination is also essential for receptor internalization upon ligand binding as exemplified by EGFR. Many cell surface receptors are internalized upon engagement with ligands, either soluble or bound to the surface of neighboring cells. The involvement of ubiquitin in these processes is just beginning to emerge and the regulation of ubiquitination is not known.

The tissue-specific expression of MARCH-IV mRNA is consistent with transcriptional silence of this gene in most tissues. Interestingly, MARCH-IV was expressed most highly in brain and in placenta both of which are immunologically privileged sites. Neurons do not express MHC I while only non-classical MHC I molecules are expressed at the maternal-fetal interface of the placenta (Furman, M. H., H. L. Ploegh and D. J. Schust, Hum. Immunol. 61:1169-1176, 2000). At present we do not know if MARCH-IV is able to downregulate non-classical HLA molecules but it is tempting to speculate that they will resist downregulation by MARCH-IV, because HLA-G also exists as a peptide-loaded protein that lacks the membrane anchor and is therefore not expected to be affected by MARCH-IV (Lee, N., A. R. Malacko, A. Ishitani, M. C. Chen, J. Bajorath, H. Marquardt and D. E. Geraghty, Immunity 3:591-600, 1995). Likely, MARCH-IV plays a role in eliminating residually expressed MHC I or MHC I induced during viral infection. In contrast, aberrant expression of MARCH-IV during carcinogenesis aids cancer cells in escaping CTL recognition similar to virally infected cells. Such MHC I downregulation is common in human cancers but the mechanism is often unknown (Garrido, F. and I. Algarra, Adv. Cancer Res. 83:117-158, 2001). Noteworthy is the fact that the majority of expressed sequence tags for MARCH-IV were isolated from cancer cells (see Unigene: Hs.66159).

As used herein, the term “antagonist” generally refers to the property of a molecule, compound or other agent to, for example, interfere with the binding of one molecule with another molecule or the stimulation of one cell by another cell either through steric hindrance, conformational alterations or other biochemical mechanism. In one regard, the term antagonist relates to the property of an agent to prevent the binding of a receptor to its ligand, e.g., the binding of MARCH with the MARCH-R, thereby inhibiting the signal transduction pathway triggered by MARCH. The term antagonist is not limited by any specific action mechanism, but, rather, refers generally to the functional property presently defined. Antagonists of the present invention include, but are not limited to, antibodies or peptides as well as other molecules that bind to the MARCH family of proteins.

Effective therapeutics depend on identifying efficacious agents devoid of significant toxicity. Compounds potentially useful in preventing or treating cancer, such as leukemia, may be screened using various assays. For instance, a candidate antagonist may first be characterized in a cultured cell system to determine its ability to neutralize MARCH in inducing carcinogenesis.

As provided herein, the compositions for and methods of treating cancer may utilize one or more antibodies used singularly, or in combination with other therapeutics to achieve the desired effects. Antibodies according to the present invention may be isolated from an animal producing the antibody as a result of either direct contact with an environmental antigen or immunization with the antigen. Alternatively, antibodies may be produced by recombinant DNA methodology using one of the antibody expression systems well known in the art. See, e.g., Harlow and Lane, Antibodies: A Laboratory Manual, Cold Spring Harbor Laboratory (1988). Such antibodies may include recombinant IgGs, chimeric fusion proteins having immunoglobulin derived sequences or “humanized” antibodies that may all be used for the treatment of cancer according to the present invention. In addition to intact, full-length molecules, the term antibody also refers to fragments thereof (such as, e.g., scFv, Fv, Fd, Fab, Fab′ and F(ab)′₂ fragments) or multimers or aggregates of intact molecules and/or fragments that bind to MARCH. These antibody fragments bind antigen and may be derivatized to exhibit structural features that facilitate clearance and uptake, e.g., by incorporation of galactose residues.

In one embodiment of the present invention, MARCH antagonists are monoclonal antibodies prepared essentially as described in Halenbeck et al. U.S. Pat. No. 5,491,065 (1997), incorporated herein by reference.

In other embodiments of the present invention, humanized anti-MARCH monoclonal antibodies are provided. The phrase “humanized antibody” refers to an antibody derived from a non-human antibody, typically a mouse monoclonal antibody. Alternatively, a humanized antibody may be derived from a chimeric antibody that retains or substantially retains the antigen binding properties of the parental, non-human, antibody but which exhibits diminished immunogenicity as compared to the parental antibody when administered to humans. The phrase “chimeric antibody,” as used herein, refers to an antibody containing sequence derived from two different antibodies (see, e.g., U.S. Pat. No. 4,816,567) which typically originate from different species. Most typically, chimeric antibodies comprise human and murine antibody fragments, generally human constant and mouse variable regions.

Because humanized antibodies are less immunogenic in humans than the parental mouse monoclonal antibodies, they can be used for the treatment of humans with far less risk of anaphylaxis. Thus, these antibodies may be preferred in therapeutic applications that involve in vivo administration to a human.

Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. These methods are disclosed in, e.g., Jones et al., Nature 321:522-525, 1986; Morrison et al., Proc. Natl. Acad. Sci., U.S.A., 81:6851-6855, 1984; Morrison and Oi, Adv. Immunol., 44:65-92, 1988; Verhoeyer et al., Science 239:1534-1536, 1988; Padlan, Molec. Immun. 28:489-498, 1991; Padlan, Molec. Immunol. 31(3):169-217, 1994; and Kettleborough, C. A. et al., Protein Eng. 4(7):773-83, 1991, each of which is incorporated herein by reference.

The phrase “complementarity determining region” refers to amino acid sequences which together define the binding affinity and specificity of the natural Fv region of a native immunoglobulin binding site. See, e.g., Chothia et al., J. Mol. Biol. 196:901-917, 1987; Kabat et al., U.S. Dept. of Health and Human Services NIH Publication No. 91-3242, 1991. The phrase “constant region” refers to the portion of the antibody molecule that confers effector functions. In the present invention, mouse constant regions are substituted by human constant regions. The constant regions of the subject humanized antibodies are derived from human immunoglobulins. The heavy chain constant region can be selected from any of the five isotypes: alpha, delta, epsilon, gamma or mu.

One method of humanizing antibodies comprises aligning the non-human heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region which disturb the structure of the CDRs until the predicted conformation of the humanized sequence model closely approximates the conformation of the non-human CDRs of the parent non-human antibody. Such humanized antibodies may be further derivatized to facilitate uptake and clearance, e.g., via Ashwell receptors. See, e.g., U.S. Pat. Nos. 5,530,101 and 5,585,089, which patents are incorporated herein by reference.

Humanized antibodies to the MARCH family of proteins can also be produced using transgenic animals that are engineered to contain human immunoglobulin loci. For example, WO 98/24893 discloses transgenic animals having a human Ig locus wherein the animals do not produce functional endogenous immunoglobulins due to the inactivation of endogenous heavy and light chain loci. WO 91/741 also discloses transgenic non-primate mammalian hosts capable of mounting an immune response to an immunogen, wherein the antibodies have primate constant and/or variable regions, and wherein the endogenous immunoglobulin encoding loci are substituted or inactivated. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy chains, and express an exogenous immunoglobulin locus comprising one or more xenogeneic constant regions.

Using a transgenic animal described above, an immune response can be produced to a selected antigenic molecule, and antibody producing cells can be removed from the animal and used to produce hybridomas that secrete human monoclonal antibodies. Immunization protocols, adjuvants, and the like are known in the art, and are used in immunization of, for example, a transgenic mouse as described in WO 96/33735. This publication discloses monoclonal antibodies against a variety of antigenic molecules including IL-6, IL-8, TNFa, human CD4, L-selectin, gp39, and tetanus toxin. The monoclonal antibodies can be tested for the ability to inhibit or neutralize the biological activity or physiological effect of the corresponding protein. WO 96/3373 discloses that monoclonal antibodies against IL-8, derived from immune cells of transgenic mice immunized with IL-8, blocked IL-8 induced functions of neutrophils. Human monoclonal antibodies with specificity for the antigen used to immunize transgenic animals are also disclosed in WO 96/34096. The MARCH antagonists of the present invention are said to be immuospecific or specifically binding if they bind to MARCH with a Kₐ of greater than or equal to about 10⁴ M⁻¹, preferably of greater than or equal to about 10⁵ M⁻¹, more preferably of greater than or equal to about 10⁶ M⁻¹, and still more preferably of greater than or equal to about 10⁶ M⁻¹. Such affinities may be readily determined using conventional techniques, such as by equilibrium dialysis; by using the BIAcore 2000 instrument, using general procedures outlined by the manufacturer; by radioimmunoassay using ¹²⁵I-labeled MARCH; or by another method known to the skilled artisan. The affinity data may be analyzed, for example, by the method of Scatchard et al., Ann N.Y. Acad. Sci., 51:660, 1949. Thus, it will be apparent that preferred MARCH antagonists will exhibit a high degree of specificity for MARCH and will bind with substantially lower affinity to other molecules.

Identification of additional MARCH antagonists may be achieved by using any of a number of known methods for identifying and obtaining proteins that specifically interact with other proteins or polypeptides, for example, a yeast two-hybrid screening system such as that described in U.S. Pat. No. 5,283,173 or the equivalent may be utilized. In one embodiment of the present invention, a cDNA encoding MARCH, or a fragment thereof, may be cloned into a two hybrid bait vector and used to screen a complementary target library for a protein having MARCH binding activity.

As used herein, the term “protein” includes proteins, oligopeptides, polypeptides, peptides and the like. Additionally, the term protein may also refer to fragments, multimers or aggregates of intact molecules and/or fragments. Proteins may be naturally occurring or may be produced via recombinant DNA means or by chemical and/or enzymatic synthesis. See, e.g., Sambrook et al., Molecular Cloning: A Laboratory Manual, Cold Spring Harbor Laboratories (3rd ed. 2001).

In addition to antibodies and other proteins, this invention also contemplates alternative MARCH antagonists including, but not limited to, small molecules that are also effective in treating cancer. Such small molecules may be identified by assaying their capacity to bind to MARCH and/or to inhibit the interaction between MARCH and its receptors or targets.

Small molecule receptor inhibitors have been isolated by high-throughput screening of compounds (Landro, J. A., J. Pharmacol. Toxicol. Methods 44:273-289, 2000). The source of these compounds varies but includes collections of natural molecules (Munro, M. H. et al., J Biotechnol. 70:15-25, 1999; Harvey, A. L., TIPS 20:196-198, 1999), combinatorial chemical libraries (Floyd, C. D. et al., Prog Med. Chem. 36:91-168, 1999; Ramstrom, O. and J. M. Lehn, Natl. Rev. Drug Discov. 1:26-36, 2002 and Lehn, 2002), or synthetic peptide libraries (Shusta et al. 1999, supra). The present invention includes molecules that specifically bind and inhibit activation of the MARCH family of proteins to be used in targeting the inhibiting, internalization, and degredation of various cell surface receptors, such as leukemia, mental retardation, and L-thalassemia.

Methods for measuring the binding of MARCH with small molecules are readily available in the art and include, for example, competition assays whereby the small molecule interferes with the interaction between MARCH and its receptor, target, or an anti MARCH antibody. Alternatively, direct binding assays may be utilized to measure the interaction of a small molecule with MARCH. By way of example, an ELISA assay may be employed whereby MARCH is adsorbed onto an insoluble matrix such as a tissue culture plate or bead. A labeled MARCH or anti-MARCH antibody is blocked from binding to MARCH by inclusion of the small molecule of interest. Alternatively, the binding of a small molecule to MARCH may be determined by a fluorescence activated cell sorting (FACS) assay. By this method, cells expressing MARCH are incubated with a fluorescent tagged anti-MARCH antibody or an anti-MARCH antibody in the presence of a fluorescent tagged secondary antibody. Binding of a small molecule to MARCH may be assessed by a dose dependent decrease in fluorescence bound to the MARCH expressing cells. Similarly, direct binding of a small molecule may be assessed by labeling, e.g., radiolabeling or fluorescent tagging, the small molecule, incubating with immobilized MARCH or MARCH expressing cells and assaying for the radioactivity or fluorescence of the bound small molecule.

Screening Assays

Screening assays. Screening Assays for drugs useful in treating or preventing conditions affected by MARCH-protein-mediated endocytosis or down-regulation of cell-surface receptors (e.g., Cancer) are within the scope of the present invention. Many different assay systems (e.g., homogeneous, heterogeneous, cell-based and non-cell-based) can be designed and used to identify compounds or compositions that modulate MARCH-protein activity or MARCH-protein gene expression, and therefore, modulate, e.g., cancer. Additionally, the systems described below may be formulated into kits. To this end, the reagents (e.g., MARCH-protein or MARCH-protein-target/receptor protein or cells expressing the MARCH-protein or MARCH-protein-target/receptor proteins) are packaged in a variety of containers, e.g., vials, tubes, microtitre well plates, bottles, and the like. Other reagents can be included in separate containers and provided with the kit; e.g., positive controls samples, negative control samples, MARCH-protein or MARCH-protein-target/receptor proteins, peptides, buffers, cell culture media, antibodies, etc.

Basic design and principle of screening assays. Without being limited to the specific embodiments discussed herein, the following assays are designed to identify compounds or compositions that, among other art-recognized possibilities: bind to MARCH-protein, MARCH-protein-target/receptor or the MARCH-protein-target/receptor:MARCH-protein complex; that interfere with the interaction between MARCH-protein-target/receptor and MARCH-protein; or that effect MARCH-protein-mediated signal transduction, MARCH-protein ubiquitination activity, or MARCH-protein-target/receptor, MARCH-protein-mediated inhibition of endocytosis (e.g., MHC-1, CD4, and transferrin receptors). Those compounds identified as antagonist or agonists of the MARCH-protein-target/receptor:MARCH-protein interaction, or as modifiers of MARCH-protein-mediated signal transduction, ubiquitination activity, inhibition of endocytosis, or other MARCH-protein-mediated activities have therapeutic utility for the treatment and prevention of, e.g., cancer.

The principle of the assays to identify compounds that inhibit the MARCH-protein-target/receptor:MARCH-protein interaction involves preparing a reaction mixture containing the test compound, and functional MARCH-protein-target/receptor and MARCH-protein proteins (or a cellular preparation comprising, in part, functional MARCH-protein), and incubating the reaction mixture for a time sufficient to allow the components to interact and bind (i.e., to form a complex which can be removed and/or detected). For example, to test a compound for antagonist activity, the reaction mixture is prepared in the presence and absence of the test compound. The test compound may be initially included in the reaction mixture, or may be added at a time subsequent to the addition of MARCH-protein-target/receptor and MARCH-proteins. Control reaction mixtures are incubated without the test compound or with a control agent. The formation of any MARCH-protein-target/receptor:MARCH-protein complexes is then detected. The formation of a complex in the control reaction, but not in the reaction mixture containing the test compound, indicates that the compound antagonizes the MARCH-protein-target/receptor:MARCH-protein interaction. Additionally, complex formation within reaction mixtures containing the test compound and normal MARCH-protein-target/receptor and MARCH-proteins may also be compared to complex formation within reaction mixtures containing the test compound and derivatized or mutant MARCH-protein-target/receptor or MARCH-protein proteins.

The principle of the assays to identify compounds that modify MARCH-protein ubiquitination activity involves preparing a reaction mixture containing the test compound, and functional MARCH-protein (or a cellular preparation comprising, in part, functional MARCH-protein), and incubating the reaction mixture for a time sufficient to allow the components to interact. Control reaction mixtures are incubated without the test compound or with a control agent. The MARCH-protein ubiquitination activity is then detected. The modulation of ubiquitination activity in the reaction mixture containing the test compound, but not in the control reaction mixture, indicates that the compound modulates MARCH-protein ubiquitination activity.

The principle of the assays to identify compounds that modify MARCH-protein-mediated signal transduction, or MARCH-protein-target/receptor:MARCH-protein-mediated inhibition of endocytosis (e.g., MHC-1, CD4 and transferrin receptors) involves preparing a reaction mixture containing the test compound, and a cellular preparation comprising, in part, functional MARCH-protein, and incubating the reaction mixture for a time sufficient to allow the components to interact. Control reaction mixtures are incubated without the test compound or with a control agent. The MARCH-protein-mediated signal transduction, or MARCH-protein-target/receptor:MARCH-protein-mediated inhibition of endocytosis is then detected. The modulation of MARCH-protein-mediated signal transduction, or MARCH-protein-target/receptor:MARCH-protein-mediated inhibition of endocytosis in the reaction mixture containing the test compound, but not in the control reaction mixture, indicates that the compound modulates MARCH-protein-mediated signal transduction, or MARCH-protein-target/receptor:MARCH-protein-mediated inhibition of endocytosis.

Homogeneous and heterogeneous assay formats. Homogeneous assays are either cell-based or non-cell-based, wherein the entire reaction is carried out in a liquid phase. Heterogeneous assays are generally (but not always; e.g., immobilized cells displaying appropriate cell-surface proteins) non-cell-based, and involve anchoring, e.g., either the ligand or the receptor (or test compound and target) onto a solid phase (e.g., test tube, microtitre well, column, or a microarray-type support or “chip”) and detecting complexes anchored on the solid phase at the end of the reaction.

In either approach, the order of addition of reactants are varied to obtain different information about the compounds being tested. For example, test compounds that interfere with the ligand/receptor interaction, e.g., by competition, are identified by conducting the reaction in the presence of the test substance; i.e., by adding the test substance to the reaction mixture prior to or simultaneously with the ligand and interactive cellular or extracellular receptor. Alternatively, test compounds that disrupt preformed complexes, e.g., compounds with higher binding constants that displace one of the components from the complex, are tested by adding the test compound to the reaction mixture after ligand/receptor complexes have been formed. The various formats are described briefly below.

Screening assays for antagonist and agonists of the MARCH-protein-target/receptor: MARCH-protein interaction are conducted in either homogeneous or heterogeneous formats. Screening assays to identify compounds that modify MARCH-protein ubiquitination activity involve either cell based, or non-cell-based homogeneous or heterogeneous formats. Screening assays to identify compounds that modify MARCH-protein-mediated signal transduction, or MARCH-protein-target/receptor:MARCH-protein-mediated inhibition of endocytosis involve primarily cell-based homogeneous assays.

Heterogeneous cell-based and non-cell-based assays. In a heterogeneous assay system, either the ligand, or the interactive cellular or extracellular receptor (or test compound and target) is anchored onto a solid surface (e.g., test tube, microtitre well, column, or a microarray-type support or “chip”), while the non-anchored species is labeled, either directly or indirectly. In practice, microtitre plates are utilized. The anchored species is immobilized by non-covalent or covalent attachments. Non-covalent attachment is accomplished by coating the solid surface with a solution of the ligand, receptor proteins or cells expressing the receptor, and optionally drying. Alternatively, an immobilized antibody specific for the species to be anchored is used to anchor the species to the solid surface. The surfaces may be prepared in advance.

In the case of ligand:receptor interactions, the assay is conducted by exposing the binding partner of the immobilized species to the coated surface with or without the test compound. Un-reacted components are removed (e.g., by washing) after the reaction is complete, and any complexes formed will remain immobilized on the solid surface.

Detection of anchored complexes are accomplished in a number of ways not limited to the following. First, where the non-immobilized species is pre-labeled, the detection of immobilized label indicates that complexes were formed. Second, where the non-immobilized species is not pre-labeled, an indirect label is used to detect anchored complexes; e.g., using a labeled antibody specific for the initially non-immobilized species (the antibody, in turn, may be directly labeled or indirectly labeled with a labeled anti-Ig antibody). Depending upon the order of addition of reaction components, test compounds which inhibit complex formation or which disrupt preformed complexes are detected.

Alternatively, the reaction can be conducted in a liquid phase in the presence or absence of the test compound, the reaction products separated from unreacted components, and complexes then heterogeneously detected by, e.g., using an immobilized antibody specific for one of the binding components to anchor any complexes formed in solution, and detecting such anchored complexes using another labeled antibody specific for the other binding partner. Test compounds that inhibit complex formation or that disrupt preformed complexes are identified, depending upon the order of reactant addition in the initial homogeneous liquid phase.

For example, in a heterogeneous assay, involving a ligand and a receptor, the ligand or receptor proteins are first prepared for immobilization using routine recombinant DNA techniques. For example, the ligand gene coding region is fused to a glutathione-S-transferase (GST) gene using a fusion vector, such as pGEX-5X-1, in such a manner that its binding activity is maintained in the resulting fusion protein. The interactive receptor is purified and used to raise a monoclonal antibody that can be labeled with the radioactive isotope ¹²⁵I. The GST-ligand fusion protein is anchored to glutathione-agarose beads. The interactive receptor is then added in the presence or absence of the test compound in a manner that allows interaction and binding to occur. Unbound material is washed away at the end of the reaction period, and the labeled monoclonal antibody is added to the system and allowed to bind to the complexed components. The ligand/receptor interaction is detected by measuring the amount of radioactivity that remains associated with the glutathione-agarose beads. A successful inhibition of the interaction by the test compound results in a decrease in measured radioactivity.

Alternatively, the GST-ligand fusion protein and the interactive receptor are mixed together in liquid in the absence of the solid glutathione-agarose beads. The test compound is added either during or after the species are allowed to interact. This mixture is then added to the glutathione-agarose beads and unbound material is washed away. The extent of inhibition of the ligand/receptor interaction is detected by adding the labeled antibody and measuring the radioactivity associated with the beads.

In the case of ubiquitination activity assays, the assay is conducted by exposing the MARCH-protein, immobilized to the coated surface, to a reaction mixture containing the test compound and a ubiquitination substrate, and incubating the reaction mixture for a time sufficient to allow the components to interact. Ubiquitination activity is detected, based on reaction of the substrate. Ubiquitination assays, both heterogeneous and heterogeneous, are well known and familiar to practitioners of ordinary skill in the relevant art.

Accordingly, the present invention provides cell-based and a non-cell based heterogeneous assays, wherein the recombinantly expressed MARCH-protein, polypeptide or fusion protein, or MARCH-protein-target/receptor:MARCH-protein complex, or cells expressing MARCH-protein are attached to a solid substrate (e.g., test tube, microtitre well, column, or a microarray-type support or “chip”). The test compounds are then assayed for their ability to either bind to the immobilized MARCH-protein protein, polypeptide or fusion protein, or to the MARCH-protein-target/receptor:MARCH-protein complex, or for their ability to antagonize or promote the binding of MARCH-protein-target/receptor protein, polypeptide or fusion protein to the immobilized MARCH-protein, polypeptide or fusion protein.

Thus, the screens are designed to identify compounds that mimic the interaction between MARCH-protein and MARCH-protein ligands, such as MARCH-protein-target/receptor. In such screens, the test compounds are labeled, and are assayed for their ability to bind to immobilized MARCH-protein protein, polypeptide or fusion protein. In another aspect of the invention the screens are designed to identify compounds that antagonize the interaction between MARCH-protein and MARCH-protein ligands, such as MARCH-protein-target/receptor. In such screens, the MARCH-protein ligand is labeled and test compounds are assayed for their ability to antagonize the binding of labeled ligand to MARCH-protein. In yet another aspect of the invention, the assays are designed to identify compounds that modulate ubiquitination activity.

Homogeneous cell-based and non-cell-based assays. Homogeneous assays are either cell-based or non-cell-based, wherein the entire reaction is carried out in a liquid phase. Cell-based assays. A homogeneous, cell-based assay system is used to screen for compounds that modulate the activity of MARCH-protein to identify compounds for the treatment of, e.g., cancer. To this end, cells that endogenously express MARCH-protein and/or MARCH-protein-target/receptor are used to screen for compounds. Alternatively, cell lines, such as 293 cells, COS cells, CHO cells, MCF-7 cells, Hs578T cells, A431 cells, fibroblasts, and the like, are genetically engineered to express MARCH-protein or MARCH-protein-target/receptor are used for screening purposes. Preferably, host cells genetically engineered to express a functional MARCH-protein-target/receptor protein that responds to MARCH-protein or MARCH-protein peptides or fusion proteins, are used as an endpoint in the assay. Such end points, for example, are provided by measurement of art-recognized changes in chemical, physiological, biological, or phenotypic properties, such as MARCH-protein-target/receptor-mediated signal transduction (based on, e.g., measurement of phosphorylation or activation of an intracellular protein (e.g., PAK; p21-activated kinase), organization of the actin cytoskeleton, gene transcription (e.g., a “reporter” gene), lipid metabolism, vesicle trafficking or cellular transformation (e.g., ras-mediated)), MARCH-protein-target/receptor:MARCH-protein-mediated inhibition of endocytosis (based on measurement of, e.g., MHC-1, CD4 or transferrin receptor endocytosis, or measurement of vesicle formation), MARCH-protein ubiquitination activity, receptor up- or down-regulation, cell motility (based on measurement of lamellipodia formation, or of organization of the actin cytoskeleton) or cell growth rate.

Preferably, for screening assays, the host cells expressing functional MARCH-protein or MARCH-protein-target/receptor proteins should provide for a significant MARCH-protein-based end-point response, preferably greater than 5-fold induction over background. Host cells preferably possess a number of characteristics, depending on the readout, to maximize the MARCH-protein-based inductive response.

For example, for detecting induction of a CRE “reporter” gene as part of a reporter gene-based assay, a set of assay conditions comprising: (a) a low natural level of cAMP, (b) a high level of adenylyl cyclase, (c) a high level of protein kinase A, (d) a low level of phosphodiesterases, and (e) a high level of cAMP response element binding protein is advantageous. In addition, alternative pathways for induction of the CRE reporter are eliminated to reduce basal levels.

To increase MARCH-protein-based responses, host cells are engineered to express a greater amount of favorable factors (e.g., enhanced or constitutive MARCH-protein and/or MARCH-protein-target/receptor expression when screening for compounds that act as MARCH-protein antagonists) or a lesser amount of unfavorable factors (e.g., enhanced MARCH-protein-target/receptor expression when screening for MARCH-protein agonists).

In utilizing such cell systems, the cells expressing the MARCH-protein or MARCH-protein-target/receptor proteins are exposed to a test compound or controls (and, in the case of ubiquitination assays, a ubiquitination substrate). After exposure, the cells are assayed to measure the expression and/or activity of components of the MARCH-protein-target/receptor:MARCH-protein signal transduction pathway. Alternatively, the activity of the signal transduction pathway itself is assayed. Alternatively, MARCH-protein ubiquitination activity is assayed.

For example, after exposure, cell lysates are assayed, e.g., for calcium mobilization, induction of cAMP, or modulation of protein tyrosine kinase (PTK), serine/threonin kinases (e.g., PAK (“p21-activated kinase”)), MARCH-protein ubiquitination activity, g-proteins, Ras, PKA, RAP1, B-Raf, Mek, or MAPK activity or phosphorylation, or for the phosphorylation or activation of any other intracellular protein known in the art to be involved in the particular signal transduction pathway in question. Activation or deactivation of particular signal transduction pathways and upstream activators can lead to activation or deactivation of many transcription factors, which in turn can mediate, among other things, cellular proliferation, differentiation, quiescence or death.

This is exemplified, e.g., in the case of neuronal differentiation of PC12 cells, by PKA's phosphorylation of active sites on a small GTP-binding protein, Rap 1, and subsequent activation of a serine/threonin kinase, B-Raf and the MAPK kinase, Mek which leads to activation of MAPK. This same pathway has been implicated in the NE differentiation of specific prostate cancer cell lines, such as LNCaP and PC-3M.

In screening for compounds that act as antagonists of MARCH-protein-target/receptor or the MARCH-protein-target/receptor:MARCH-protein interaction, it is advantageous to over-express MARCH-protein to test for inhibition of signal transduction by the test compound as compared to controls. In screening for compounds that may act as agonists of MARCH-protein-target/receptor or the MARCH-protein-target/receptor:MARCH-protein interaction, it is advantageous to use cell lines that express little or no MARCH-protein to test for activation of MARCH-protein-target/receptor/MARCH-protein-mediated endocytosis or signal transduction by the test compound as compared to controls.

MARCH-protein has been shown herein to mediate endocytosis of, for example, MHC-1, CD4 and and transferrin receptors Example 3 and FIG. 8). Thus, in further embodiments, test compounds are selected based on their ability to regulate or inhibit endocytosis of receptor or adhesion molecules.

Non-cell-based assays. In addition to homogeneous cell-based assays, homogeneous non-cell-based assay systems are used to identify compounds that modulate ubiquitination activity, or interact with, e.g., bind to MARCH-protein, MARCH-protein-target/receptor or to the MARCH-protein-target/receptor:MARCH-protein complex. Such compounds act as agonists or antagonists of MARCH-protein activity and are used in the treatment of, e.g., cancer. Recombinant MARCH-protein proteins, polypeptides, fusion proteins or soluble MARCH-protein proteins are expressed and utilized (with or without MARCH-protein-target/receptor proteins, polypeptides or fusion proteins) in non-cell based assays to identify compounds that bind to MARCH-protein, MARCH-protein-target/receptor or the MARCH-protein-target/receptor: MARCH-protein complex. Alternatively, polypeptides corresponding to one or more MARCH-protein or MARCH-protein-target/receptor domains, or fusion proteins containing one or more of the MARCH-protein or MARCH-protein-target/receptor domains are used in non-cell based assay systems to identify compounds that modulate ubiquitination activity, or that bind to MARCH-protein, MARCH-protein-target/receptor or to the MARCH-protein-target/receptor:MARCH-protein complex. Compounds identified are therapeutically useful to modulate MARCH-protein-target/receptor:MARCH-protein-mediated activities, such as modulation of cell growth, cell motility, signal transduction, or of inhibition of endocytosis.

For example, assays based on fluorescent reporter moieties are examples of homogeneous non-cell-based assays. Such an assay, for example, comprise a fluorescent “reporter moiety” and a “quencher moiety,” each covalently bound to linker moieties (e.g., phosphoramidites, or other appropriate linkers available in the art) whereby each is attached to one member of the interacting protein pair at issue (e.g., a reporter moiety attached to the ligand, and a quencher moiety attached to the interacting receptor). Examples of suitable reporter and quencher molecules are 5′ fluorescent reporter dyes 6FAM (“FAM”; 2,7 dimethoxy-4,5-dichloro-6-carboxy-fluorescein), TET (6-carboxy-4,7,2′,7′-tetrachlorofluorescein), and the 3′ quencher dye TAMRA (6-carboxytetramethylrhodamine) (Livak et al., PCR Methods Appl. 4:357-362, 1995; Gibson et al., Genome Res. 6:995-1001; and 1996; Heid et al., Genome Res. 6:986-994, 1996).

When the reporter and quencher moieties are in close proximity, the reporter moiety emission is transferred efficiently to the quenching moiety, and the fluorescent-emission spectrum (e.g., at 518 nm) is quenched or masked. However, on separation, the reporter moiety emission is no longer transferred efficiently to the quenching moiety, resulting in an increase of the reporter moiety fluorescent-emission spectrum. Typically, fluorochromes are chosen such that the fluorescent intensity of the quenching moiety (e.g., TAMRA), changes very little over the course of the reaction. Several factor influence the efficiency of such fluorescent-based assays, including magnesium and salt concentrations, reaction conditions (time and temperature), protein sequence, size and composition. Optimization of these factors to produce the optimum fluorescence intensity for a given genomic locus is obvious to one skilled in the relevant art.

Accordingly, in one embodiment embraced by the present invention, a preformed MARCH-protein-target/receptor:MARCH-protein complex is prepared in which either MARCH-protein-target/receptor or MARCH-protein (or both) is labeled with a suitable fluorescent tag, whereby the signal generated by the label or tag is quenched due to complex formation (see, e.g., U.S. Pat. No. 4,109,496). The addition of a test substance that competes with and displaces one of the species from the preformed complex results in the generation of a signal above background. In this way, test substances that antagonize the MARCH-protein-target/receptor: MARCH-protein interaction are identified.

In another embodiment, cell-based or non-cell-based assays are used to screen for modulators of ubiquitination activity.

MARCH antagonists of the present invention include, where applicable, functional equivalents. For example, molecules may differ in length, structure, components, etc. but may still retain one or more of the defined functions. More particularly, functional equivalents of the antibodies, antibody fragments or peptides of the present invention may include mimetic compounds, i.e., constructs designed to mimic the proper configuration and/or orientation for antigen binding.

Preferred MARCH antagonists may optionally be modified by addition of side groups, etc., e.g., by amino terminal acylation, carboxy terminal amidation or by coupling of additional groups to amino acid side chains. Antagonists may also comprise one or more conservative amino acid substitutions. By “conservative amino acid substitutions” is meant those changes in amino acid sequence that preserve the general charge, hydrophobicity/hydrophilicity and/or steric bulk of the amino acid substituted. For example, substitutions between the following groups are conservative: Gly/Ala, Val/Ile/Leu, Asp/Glu, Lys/Arg, Asn/Gln, Ser/Cys/Thr, and Phe/Trp/Tyr. Such modifications will not substantially diminish the efficacy of the MARCH antagonists and may impart such desired properties as, for example, increased in vivo half life or decreased toxicity.

Antisense oligomers. MARCH-specific oligomers and corresponding peptide nucleoic acid oligomers (PNA), and including antisense oligomers, are encompassed within the scope of the present invention.

Examples of inventive oligonucleotides of length X (in nucleotides), as indicated by polynucleotide positions with reference to, e.g., SEQ ID NO:44, include those corresponding to sets (sense and antisense sets) of consecutively overlapping oligonucleotides of length X, where the oligonucleotides within each consecutively overlapping set (corresponding to a given X value) are defined as the finite set of Z oligonucleotides from nucleotide positions:
n to (n+(X−1));
where n=1, 2, 3, . . . (Y—(X−1));
where Y equals the length (nucleotides or base pairs) of SEQ ID NO:1 (4,450);
where X equals the common length (in nucleotides) of each oligonucleotide in the set (e.g., X=20 for a set of consecutively overlapping 20-mers); and
where the number (Z) of consecutively overlapping oligomers of length X for a given SEQ ID NO of length Y is equal to Y−(X−1). For example Z=4,450−19=4,431 for either sense or antisense sets of SEQ ID NO:1, where X=20.

Examples of inventive 20-mer oligonucleotides include the following set of 4,431 oligomers (and the antisense set complementary thereto), indicated by polynucleotide positions with reference to SEQ ID NO:44:
1-20, 2-21, 3-22, 4-23, 5-24, . . . 4,429-4,448, 4,430-4,449 and 4,431-4,450.

Likewise, examples of inventive 25-mer oligonucleotides include the following set of 4,426 oligomers (and the antisense set complementary thereto), indicated by polynucleotide positions with reference to SEQ ID NO:1:
1-25, 2-26, 3-27, 4-28, 5-29, . . . 4,424-4,448, 4,425-4,449 and 4,426-4,450.

The present invention encompasses, for each of SEQ ID NOS:38, 40, 42, 44, 46, 48, 50 and 52 (sense and antisense), multiple consecutively overlapping sets of oligonucleotides or modified oligonucleotides of length X, where, e.g., X=9, 10, 17, 20, 22, 23, 25, 27, 30 or 35 nucleotides.

Combinations of antagonists. Having identified more than one MARCH antagonist that is effective in an animal model, it may be further advantageous to mix two or more such MARCH antagonists together to provide still improved efficacy against cancer metastasis and/or bone loss associated with cancer metastasis. Compositions comprising one or more MARCH antagonist may be administered to persons or mammals suffering from, or predisposed to suffer from cancer.

By the present methods, compositions comprising MARCH antagonists may be administered parenterally, topically, orally or locally for therapeutic treatment. Preferably, the compositions are administered orally or parenterally, i.e., intravenously, intraperitoneally, intradermally or intramuscularly. Thus, this invention provides methods which employ compositions for administration which comprise one or more MARCH antagonists in a pharmaceutically acceptable carrier, preferably an aqueous carrier. A variety of aqueous carriers may be used, e.g., water, buffered water, 0.4% saline, 0.3% glycine and the like, and may include other proteins for enhanced stability, such as albumin, lipoprotein, globulin, etc., subjected to mild chemical modifications or the like.

MARCH antagonists useful as therapeutics for cancer will often be prepared substantially free of other naturally occurring immunoglobulins or other biological molecules. Preferred MARCH antagonists will also exhibit minimal toxicity when administered to a mammal afflicted with, or predisposed to suffer from, cancer.

The compositions of the invention may be sterilized by conventional, well known sterilization techniques. The resulting solutions may be packaged for use or filtered under aseptic conditions and lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride and stabilizers (e.g., 1-20% maltose, etc.).

The MARCH antagonists of the present invention may also be administered via liposomes. Liposomes, which include emulsions, foams, micelles, insoluble monolayers, phospholipid dispersions, lamellar layers and the like, can serve as vehicles to target the MARCH antagonists to a particular tissue as well as to increase the half life of the composition. A variety of methods are available for preparing liposomes, as described in, e.g., U.S. Pat. Nos. 4,837,028 and 5,019,369, which patents are incorporated herein by reference.

The concentration of the MARCH antagonist in these compositions can vary widely, i.e., from less than about 10%, usually at least about 25% to as much as 75% or 90% by weight and will be selected primarily by fluid volumes, viscosities, etc., in accordance with the particular mode of administration selected. Actual methods for preparing orally, topically and parenterally administrable compositions will be known or apparent to those skilled in the art and are described in detail in, for example, Remington's Pharmaceutical Science, 19th ed., Mack Publishing Co., Easton, Pa. (1995), which is incorporated herein by reference.

Determination of an effective amount of a composition of the invention to treat cancer in a patient can be accomplished through standard empirical methods which are well known in the art.

Compositions of the invention are administered to a mammal already suffering from, or predisposed to, cancer in an amount sufficient to prevent or at least partially arrest the development of cancer. An amount adequate to accomplish this is defined as a “therapeutically effective dose.” Effective amounts of a MARCH antagonist will vary and depend on the severity of the disease and the weight and general state of the patient being treated, but generally range from about 1.0 μg/kg to about 100 mg/kg body weight, with dosages of from about 10 μg/kg to about 10 mg/kg per application being more commonly used. Administration is daily, weekly or less frequently, as necessary depending on the response to the disease and the patient's tolerance of the therapy. Maintenance dosages over a prolonged period of time may be needed, and dosages may be adjusted as necessary.

Single or multiple administrations of the compositions can be carried out with the dose levels and pattern being selected by the treating physician. In any event, the formulations should provide a quantity of MARCH antagonist sufficient to effectively prevent or minimize the severity of cancer. The compositions of the present invention may be administered alone or as an adjunct therapy in conjunction with other therapeutics known in the art for the treatment of cancer metastasis and/or bone loss associated with cancer metastasis.

Methods Used in the Examples Herein Below

In the Examples 1 through 5, below, and unless otherwise indicted, Hela-Tet-Off cells were obtained from Clontech (Palo Alto, Calif.) and maintained in Dulbecco's modified Eagle medium (DMEM) (from Mediatech, Inc., Herndon, Va.) with 10% fetal calf serum (Hyclone, Logan, Utah) and 1×Pen-Strep-Glutamine (Invitrogen, Carlsbad Calif.). All transfections were done using Effectene reagent (Qiagen, Valencia, Calif.) according to the manufacturer's protocol. A plasmid containing the MARCH-I cDNA was a gift from Sumio Sugano, University of Tokyo (cDNA FLJ20668, clone KAIA585). The open reading frame from MARCH-I was amplified and a C-terminal Flag-tag added via PCR using the primers: Forward 5′-CCT AGA ATT CAC CAT GAC CAG CAG CCA CG-3′(SEQ ID NO:1), Reverse 5′-ATC GGG ATC CTC ACT TGT CAT CGT CAT CCT TGT AGT CGA CTG ATA CAA CTT C-3′ (SEQ ID NO:3). The resulting fragment was then digested with EcoRI and BamHI and ligated between the corresponding sites of the pUHD 10-1 vector.

The DNA fragment encoding the N-terminal 137 amino-acids of MARCH-I, containing the PHD domain, was amplified via PCR using the above construct as template and the above forward primer with the Reverse primer: 5′-ATC GGC GGC CGC TCA TTT CCT CCT TTC ACT T-3′ (SEQ ID NO:4). This fragment was then inserted between the EcoRI and NotI sites of the pGEX-4T-1 vector (AP Biotech).

A plasmid containing the MARCH-II cDNA was obtained via the IMAGE consortium (clone ID 24525). The MARCH-II open reading frame was amplified and a C-terminal Flag-tag added via PCR using the primers: Forward 5′-CCT AGA ATT CAC CAT GAC GAC GGG TGA C-3′(SEQ ID NO:5), Reverse 5′-ATC GGG ATC CTC ACT TGT CAT CGT CAT CCT TGT AGT CTA CTG GTG TCT CCT CTG C-3′ (SEQ ID NO:7). Upon restriction digest, the resulting fragment was inserted between the EcoRI and BamHI sites of the pUHD10⁻¹ vector. DNA coding for the N-terminal 137 amino-acids of MARCH-II, containing the PHD domain, was amplified using the above forward primer with the Reverse primer: 5-ATC GGC GGC CGC TCA TGT CCG CTT CTC CGT CC-3′ (SEQ ID NO:62). The resulting fragment was inserted between the EcoRI and NotI sites in the pGEX-4T-1 vector (AP Biotech).

N-terminal His₆-tagged B2 constructs of human ubc6 and ubc7 were constructed using the NhcI and BamII sites of pET28 (Novagen, Inc. Madison, Wis.). The human ubc7 gene is a homologue of yeast Ubnc7 and is also called UBE2G2, to distinguish it from another human E2, namely UbCH7. cDNA for human ubc7 was obtained from the IMAGE consortium (clone ID 563616), through Research Genetics (Invitrogen, Carlsbad, Calif.). The following primers were purchased from Invitrogen: (hUbc6-forward: 5′-ATA TGC TAG CGC CAT GAG GAG CAC CAG CAG TAA G-3′ (SEQ ID NO:63)), (hUbc6-REVERSE: 5′-ATA TGC ATC CTC ACT CCT GCG CGA TGC TCC TC-3′ (SEQ ID NO:64)), (hUbc7-forward: 5′-ATA TGC TAG CGC CAT GGC GGG GAC CGC GCT CAA G-3′ (SEQ ID NO:65)), (hUbc7-reverse:5′-ATA GGG ATC CTC ACA GTC CCA GAG ACT TCT GG-3′ (SEQ ID NO:66)). DNA sequencing confirmed the sequence for human ubc-7. Sequencing of the human ubc6 clone showed that it differs from the sequence with the Accession number AF296658 by having three point insertions in the DNA region corresponding to amino acids 214 to 238. The resulting frameshifted sequence, with identical point insertions, is also found in a database entry NM-058167. All three variants of human ubc6 are identical in protein sequences between amino acids 45 and 213 (using the numbering from AF296658).

In the Examples 6 through 8, below, and unless otherwise noted, the following experimental conditions were used:

Cell culture. Hela-Tet-Off cells were obtained from Clontech (Palo Alto, Calif.) and maintained in Dulbecco's modified Eagle medium (DMEM) (from Cellgro) with 10% fetal calf serum (Hyclone) and 1×Pen-Strep-Glutamine (Invitrogen).

Reagents. Conanamycin A, Leupeptin (both from Sigma) and lactacystin (Boston, Biochem) were used at final concentration of 50 nM, 0.3 mM and 20 μM, respectively. Protein A/G beads were from Santa Cruz Biotechnology.

Plasmids and cloning. A plasmid containing the MARCH-I cDNA was a gift from Sumio Sugano, University of Tokyo (cDNA FLJ20668, clone KAIA585). Plasmids containing the MARCH-II, MARCH-V, MARCH-VII, and MARCH-VIII cDNA was obtained via the IMAGE consortium (clone IDs 24525, 3905766, 3449089, and 4830278 respectively). A plasmid containing the MARCH-IV cDNA was obtained from the Kazusa DNA Research Institute (Japan) (clone KIAA1399). Each MARCH gene was amplified via PCR both with and without a C-terminal FLAG epitope tag. The PCR products were then restricted and ligated into either the pUHD10-1 or pUHG10-3 vector. Additionally, the PHD domains of MARCH-I, MARCH-II, MARCH-IV, and MARCH-VIII were amplified and cloned into the pGEX-4T-1 vector (AP Biotech). The reference numbers and cDNA sources are summarized in TABLE 1.

TABLE 1
MARCH cDNA Reference Numbers and Sources

| | | cDNA | Protein |
|---|---|---|---|
| | | Accession | Accession |
| Protein | cDNA Source | numbers | numbers |
| MARCH I | Sumio Sugano, University of Tokyo; | | BAA91319 |
| | FLJ20668 cDNA; KAIA585 | | (SEQ ID NO: 54) |
| | | AL713759 | CAD2852.1 |
| | | (SEQ ID NO: 38) | (SEQ ID NO: 39): |
| MARCH II | IMAGE consortium; 24525 cDNA | | AAF36360 |
| | | | (SEQ ID NO: 55) |
| | | AF151074 | AAF36160.1 |
| | | (SEQ ID NO: 40) | (SEQ ID NO: 41) |
| MARCH III | | | XP_055276 |
| | | | (SEQ ID NO: 56) |
| | | XM_055278 | XP_055278.1 |
| | | (SEQ ID NO: 42) | (SEQ ID NO: 43) |
| MARCH IV | Kazusa DNA Research Institute | | XP_046685 |
| | (Japan); KIAA1399 cDNA | | (SEQ ID NO: 57) |
| | | AB037820 | BAA92637.1 |
| | | (SEQ ID NO: 44) | (SEQ ID NO: 45) |
| MARCH V | IMAGE consortium; 3905766 | | BAA91173 |
| | cDNA | | (SEQ ID NO: 58) |
| | | NM_017824 | NP_060294.1 |
| | | (SEQ ID NO: 46) | (SEQ ID NO: 47) |
| MARCH VI | | | BAA25523 |
| | | | (SEQ ID NO: 59) |
| | | AB011169 | BAA25523.1 |
| | | (SEQ ID NO: 48) | (SEQ ID NO: 49) |
| MARCH VII | IMAGE consortium; 3449089 | | AAH03404 |
| | cDNA | | (SEQ ID NO: 60) |
| | | BC003404 | AAH03404.1 |
| | | (SEQ ID NO: 50) | (SEQ ID NO: 51) |
| MARCH | IMAGE consortium; 4830278 | | AAH25394 |
| VIII | cDNA | | (SEQ ID NO: 61) |
| | | BC025394 | AAH25394.1 |
| | | (SEQ ID NO: 52) | (SEQ ID NO: 53) |

All primers used in cloning and their applicable restriction sites are listed herein below:

MARCH-I:

(SEQ ID NO:1)

Forward (EcoRI)
5′-CCT AGA ATT CAC CAT GAC CAG CAG CCA CG-3′;

(SEQ ID NO:2)

Reverse (BamHI)
5′-ATC GGG ATC CTC AGA CTG ATA CAA CTT C-3′;

(SEQ ID NO:3)

Reverse + FLAG (BamHI)
5′-ATC GGG ATC CTC ACT TGT CAT CGT CAT CCT TGT AGT

CGA CTG ATA CAA CTT C-3′;
(SEQ ID NO:4)

Reverse for RING-CH (NotI)
5′-ATC GGC GGC CGC TCA TTT CCT CCT TTC ACT T-3′;
MARCH-II:

(SEQ ID NO:5)

Forward (EcoRI)
5′-CCT AGA ATT CAC CAT GAC GAC GGG TGA C-3′;

(SEQ ID NO:6)

Reverse (KpnI)
5′-ATC GGG ATC CTC ATA CTG GTG TCT CCT CTG C-3′;

(SEQ ID NO:7)

Reverse + FLAG (KpnI)
5′-ATC GGG ATC CTC ACT TGT CAT CGT CAT CCT TGT AGT

CTA CTG GTG TCT CCT CTG C-3′;
(SEQ ID NO:8)

Reverse for RING-CH (NotI)
5′-ATC GGC GGC CGC TCA TGT CCG CTT CTC CGT CC-3′;
MARCH-III:

(SEQ ID NO:9)

Forward (EcoRI)
5′-ATC GGA ATT CAC CAT GAC AAC CAG CCG C-3′;

(SEQ ID NO:10)

Reverse (BamHI)
5′-ATC GGG ATC CTC AAA CAA CTG TCT CCT-3′;

(SEQ ID NO:11)

Reverse + FLAG (BamHI)
5′-ATC GGG ATC CTC ACT TGT CAT CGT CAT CCT TGT AGT

CAA CAA CTG TCT CCT-3′;

MARCH-IV:

(SEQ ID NO:12)

Forward (EcoRI)
5′-ATC GGA ATT CAC CAT GCA CGG CGA CCC CC-3′;

(SEQ ID NO:13)

Forward full length (EcoRI)
5′-ATC GGA ATT CAC CAT GCT GAT GCC CCT GTG TGG-3′;

(SEQ ID NO:14)

Reverse (KpnI)
5′-ATC GGG TAC CTC ACA CTG TCG TGA CTC-3′;

(SEQ ID NO:15)

Reverse + FLAG (KpnI)
5′-ATC GGG TAC CTC ACT TGT CAT CGT CAT CCT TGT AGT

CCA CTG TCG TGA CTC-3′;
(SEQ ID NO:16)

Reverse for RING-CH (NotI)
5′-TCG ATA CGC GGC CGC TCA CTG AAC CTT CTC AAT

GAC-3′;
(SEQ ID NO:17)

Forward (pUHG10-3) (BamHI)
5′-GGA TCC ACC ATG CAC GGC GAC CCC-3′;

(SEQ ID NO:18)

Reverse (pUHG10-3) (XbaI)
5′-CGC GTC TAG AGC GTC ATC ACA CTG TCG TGA CTC

T-3′;
(SEQ ID NO:19)

MARCH-V: Forward (KpnI)
5′-ATC GGG TAC CAC CAT GCC GGA CCA AGC C-3′;

(SEQ ID NO:20)

Reverse (BamHI)
5′-ATC GGG ATC CTC ATG CTT CTT CTT GTT CT-3′;

(SEQ ID NO:21)

Reverse + FLAG (BamHI)
5′-ATC GGG ATC CTC ACT TGT CAT CGT CAT CCT TGT AGT

CTG CTT CTT CTT GTT CT-3′;

MARCH-VIII:

(SEQ ID NO:22)

Forward (EcoRI)
5′-ATC GGA ATT CAC CAT GAG CAT GCC ACT GC-3′;

(SEQ ID NO:23)

Reverse (KpnI)
5′-ATC GGG TAC CTC AGA CGT GAA TGA TTT C-3′;

(SEQ ID NO:24)

Reverse + FLAG (KpnI)
5′-ATC GGG TAC CTC ACT TGT CAT CGT CAT CCT TGT AGT

CGA CGT GAA TGA TTT C-3′;
(SEQ ID NO:25)

Reverse for RING-CH (NotI)
5′-TCG ATA CGC GGC CGC TCA CTT CCT GCG CTC GCT GGA

CGT CAT CT-3′.

Flow cytometry. Cells were removed from tissue culture dishes with 0.05% Trypsin-EDTA (Invitrogen), washed with ice-cold PBS, and incubated with appropriate antibody for 30 minutes at 4° C. The cells were washed with ice-cold PBS and either resuspended in ice-cold PBS or incubated with PE-conjugated anti-mouse secondary antibody (DAKO) and washed again before analysis with a BD Biosciences (San Jose, Calif.) FACScalibur flow cytometer.

Uptake Assay. Uptake of MHC I was measured as previously described (Ishido et al., 2000b, supra). In short, HeLa cells were transfected with MARCH-IV. Twenty four hours post-transfection cells were washed and an anti-MHC I antibody allowed to bind at 4° C. Cells were then either fixed immediately or transferred to 37° C. for 120 minutes to allow uptake of antibody bound glycoproteins. Antibody bound to glycoprotein was visualized using an Alexaflour:594-conjugated secondary antibody.

Immune fluorescence. 1.5×10⁴ cells were plated on 15 mm coverslips (Fisher) and allowed to adhere overnight prior to transfection. Following transfection, cells were washed with PBS, fixed with 2% paraformaldehyde for 20 minutes at room temperature, and permeabilized with 0.2% Triton X-100 for 3 minutes at room temperature. Non-specific binding sites were blocked with 3% BSA and 0.5% fish gelatin in PBS for 30 minutes at 37° C. The fixed cells were incubated overnight at 37° C. with primary antibody diluted in blocking solution. Secondary and conjugated antibodies were diluted in blocking solution and incubated with the cells for at least 30 min at 37° C. Cells were washed 6 times with PBS between all antibody treatments. Slides were fixed a second time in 2% paraformaldehyde after the final antibody treatment and washed twice with PBS. Coverslips were then mounted on slides and covered with Vectashield H-1200+DAPI (Vector Laboratories, Burlingame, Calif.). Slides were visualized using an Axioskop 2 light microscope (Zeiss, Thornwood, NY). All pictures were taken in monochrome; contrast enhanced, and artificially colored using Openlab software (Improvision, Lexington, Mass.). Primary antibodies used for immunofluorescence were as follows: anti-EEA-1 (BD Transduction Labs (San Diego, Calif.) at 1:200), anti-Golgin-97 (Molecular Probes (Eugene, Oreg.) at 1:100), anti-Calnexin (Stressgen (British Columbia, Canada) at 1:500), anti-Flag:FitC (Sigma at 1:400), anti-LAMP-I (Univserity of Iowa) at 1:1000).

Metabolic labeling, immunoprecipitation and Western blotting. HeLa cells were grown to 80% confluency in 100 mm tissue culture dishes and transfected as above. Cells were incubated in serum-free and methionine-free medium for 30 minutes, and metabolically labeled with ³⁵S-cysteine/³⁵S-methionine (250 μCi, Amersham) for the indicated time. After labeling, cells were washed twice with PBS and either lysed immediately in PBS containing 1% Triton X-100 and protease inhibitors (Roche, Indianapolis, Ind.), or the label was chased with excess (2 mM) unlabeled cysteine and methionine in the presence or absence of specific inhibitors for the indicated time. The cell lysates were precleared with protein A/G agarose beads and incubated with 3 μg of antibody for one hour and followed by two hours incubation with protein A/G beads. Immunoprecipated proteins were washed five times with 0.1% Triton X-100 in PBS. All samples were boiled in SDS buffer and analyzed by SDS-PAGE gel electrophoresis. Where indicated, samples were treated with Endoglycosidase H (Roche) according to manufacturer's instructions. Briefly, samples were initially denatured at 100° C. in 40 μl of 0.3% SDS, 150 mM 2-mercaptoethanol, 50 mM sodium acetate, pH 5.5 and incubated with 4 milli-Units of Endo H overnight at 37° C. All samples were boiled in SDS buffer and analyzed by SDS-PAGE gel electrophoresis. Gels were fixed, dried and exposed to Kodak BioMax MR film. Western blotting was accomplished using the WesternBreeze Chemiluminescent Detection System (Invitrogen) following Semi-dry transfer to PVDF membranes (Millipore, Bedford, Mass.).

Ubiquitination assay. UbCH2 and UbCH3 were obtained from Boston Biochem. (His)₆-UbCH5a and GST fusion proteins were expressed in BL21-DE3 cells (Stratagene) and BL21 cells (Amersham), respectively, and purified as previously described (Boutell, C., S. Sadis and R. D. Everett, J. Virol. 76:841-850, 2002; Tiwari, S. and A. M. Weissman, J. Biol. Chem. 276:16193-16200, 2001). Purified E3 proteins were stored at 4° C., and E2 proteins were stored at −80° C. In vitro reaction mixtures (20 μl total) included: 50 nM rabbit E1 (Boston Biochem), 28 μM Ubiquitin, 5 mM ATP, 50 mM Tris-HCl pH 7.5, 50 mM NaCl, and 2 mM DTT. E2 enzymes were used at concentrations varying from 0.1 to 2.3 μM, and E3 concentrations ranged from 3 to 30 μM. After 90 minutes of incubation at 30° C., reactions were stopped by boiling in SDS sample buffer. Samples were electrophoresed through 12% polyacrylamide SDS gels and blotted using anti-ubiquitin mouse antibody, P4D1 (Santa Cruz Biotech).

Real-Time PCR. Short (50-100 bp) fragments from MARCH-I, MARCH-II, MARCH-IV or MARCH-VIII cDNA were amplified via PCR using the primers listed below. The PCR reaction was carried out in the following buffer: 1×SYBR Green PCR Buffer (PE Biosystems), 3 mM MgCl₂, 0.8 mM dNTP's, 0.625 U Amplitaq Gold (PE Biosystems), 0.01 μl Amperase (PE Biosystems), and 50 nM primers. 23 μl buffer was added to 2 μl template cDNA and run under the following conditions: 95° C. for 10 min, followed by 40 cycles of 95° C. for 15 seconds, 60° C. for 1 minute. Amplification was tracked via CYBR-Green (PE Biosystems) incorporation using an ABI-PRISM 7700 Sequence Detection System (Applied Biosystems, Foster City, Calif.). Absolute standards were generated using known quantities of cloned MARCH cDNA's.

MARCH-I:

(SEQ ID NO:26)

Forward 5′-CCA CAA ACA GGT GCA AAT TCA-3′;

(SEQ ID NO:27)

Reverse 5′-TCC CAA CAG GTT CCA TCA GAC-3′;
MARCH-II:

(SEQ ID NO:28)

Forward 5′-GCT GCG ACA TGG TGT GTT TC-3′;

(SEQ ID NO:29)

Reverse 5′-AGG CAC AAC CAG CCT GAG AT-3′;
MARCH-IV

(SEQ ID NO:30)

Forward 5′-TGG CTC ATC TGG TCA ACT TTC-3′;

(SEQ ID NO:31)

Reverse 5′-TGT CTT GTC ATA GTT CAG CAC-3′;
MARCH-VIII:

(SEQ ID NO:32)

Forward 5′-TCC TCC GTC AGC ATC AGC T-3′;

(SEQ ID NO:33)

Reverse 5′-GGC TGG ATG GCG TGA TAG A-3′.

The following examples are offered by way of illustration not limitation.

### EXAMPLE 1

### (MARCH-I and MARCH-II were Found to be Expressed in a Tissue-Specific Manner)

A 67 bp fragment from the MARCH-I ORF was amplified via PCR using the following primers: Forward 5′-AGA ATC TGT CAC TGC GAA GGG-3′ (SEQ ID NO:34); and Reverse 5′-AGT GTC CCA GTG CAG CGA C-3′ (SEQ ID NO:35). A 63 bp fragment from the MARCH-II ORF was amplified via PCR using the primers: Forward 5′-GCT GCG ACA TGG TGT GTT TC-3′ (SEQ ID NO:36); and Reverse 5′-AGG CAC AAC CAG CCT GAG AT-3′ (SEQ ID NO:37). PCR reactions were carried out in the following buffer: 1×SYBR Green PCR Buffer (PE Biosystems), 3 mM MgCl₂, 0.8 mM dNTP's, 0.625 U Amplitaq Gold (Applied Biosystems, Foster City, Calif.), 0.01 μl Amperase (Roche), and 50 nM primers. A volume of 23 μl buffer was added to 2 μl template, and run under the following conditions: 95° C. for 10 min, followed by 45 cycles of 60° C. for 1 min, 95° C. for 1 min. Amplification was tracked via CYBR-Green (PE Biosystems) incorporation using an ABI-PRISM 7700 Sequence Detection System (Applied Biosystems, Foster City, Calif.). Absolute standards were generated using a known quantity MARCHI-Flag/pUHD10-1 or MARCHII-Flag/pUHD10-1.

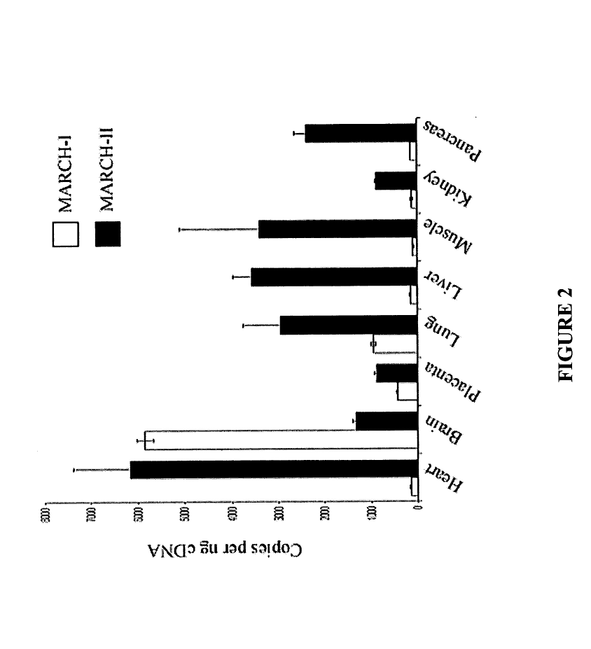

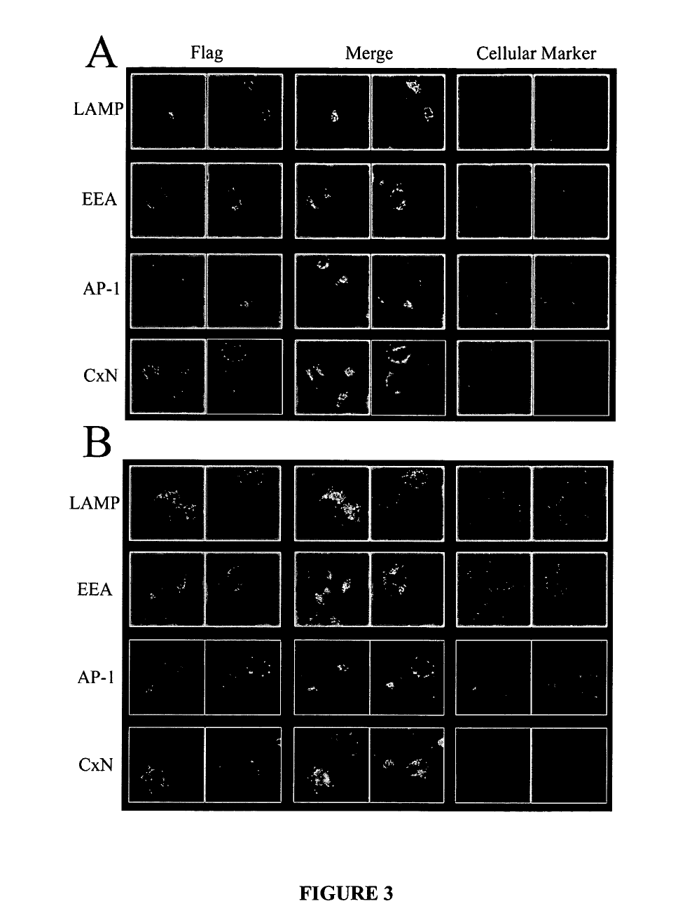

To determine if the human proteins were functionally related to the viral homologs, the MARCH-I and MARCH-II genes were selected for further studies. Both MARCH-I and II encode two predicted transmembrane domains. Expressed sequence tags corresponding to MARCH-I and II have been isolated from a variety of cDNA libraries without any clear indication of tissue specificity. To investigate whether the proteins are ubiquitously expressed or tissue-specific, Real-time RT-PCR was performed on cDNA from a number of human tissue samples. As shown in FIG. 2, MARCH-II was found in most tissues, whereas expression of MARCH-I was highest in brain tissues.

To further investigate whether MARCH protein expression is ubiquitous or tissue-specific, real-time RT-PCR was performed using primers specific for MARCH-I, II, IV and VIII on a panel of human tissue samples (FIG. 7C). The most abundant expression was observed for MARCH-II, which was expressed in every tissue analyzed at high copy numbers (note the different scale). The highest level of MARCH-IIexpression was observed in the heart sample.

In contrast, transcripts for the other three MARCH-proteins tested were detected in only some tissues and at relatively lower levels. These data indicate a cell-specific rather than wide-spread expression of MARCH-I, IV and VIII.

### EXAMPLE 2

### (Subcellular Localizations of MARCH-I and MARCH-II were Determined)

In this EXAMPLE, 1.5×10⁴ cells were plated on 15 mm coverslips (Fisher) and allowed to adhere overnight prior to transfection. Following transfection, cells were washed with PBS, fixed with 2% paraformaldehyde for 20 minutes at room temperature, and permeabilized with 0.2% Triton X-100 for 3 minutes at room temperature. Non-specific binding sites were blocked with 3% BSA and 0.5% fish gelatin in PBS for 30 minutes at 37° C. The fixed cells were incubated overnight at 37° C. with primary antibody diluted in blocking solution. Secondary and conjugated antibodies were diluted in blocking solution and incubated with the cells for at least 30 min at 37° C. Cells were washed 6-times with PBS between all antibody treatments. Slides were fixed a second time in 2% paraformaldehyde after the final antibody treatment and washed twice with PBS. Coverslips were then mounted on slides and covered with Vectashield H-1200+DAPI (Vector Laboratories, Burlingame, Calif.). Slides were visualized using an Axioskop 2 light microscope (Zeiss, Thornwood, N.Y.). All pictures were taken in monochrome; contrast enhanced, and false colored using Openlab software (Improvision, Lexington, Mass.). Primary antibodies used for immunofluorescence were as follows: anti-EEA-1 (BD Transduction Labs (San Diego, Calif.) at 1:200), anti-Golgin-97 (Molecular Probes (Eugene, Oreg.) at 1:100), anti-Calnexin (Stressgen (British Columbia, Canada) at 1:500), anti-Flag:FitC (Sigma at 1:400), anti-LAMP-I (University of Iowa) at 1:1000).

The prediction of two transmembrane domains for MARCH-I and MARCH-II suggests that both are transmembrane proteins. To examine this, the subcellular localization of both proteins was determined. cDNA clones coding for MARCH-I and II were obtained (TABLE 1) and cloned into the mammalian expression vector pUHD10-1. To allow their detection a sequence coding for the FLAG-epitope was fused to the 3′-end of both coding regions. Similar epitope fusion constructs were previously shown not to affect the function of homologous viral proteins (Coscoy, L., and D. Ganem, 2001, supra; Paulson et al., 2001, supra). The resulting constructs were transfected into HeLa cells. Transfected cells were permeabilized and stained with antibodies to the FLAG epitope as well as antibodies to various markers of subcellular compartments including: lysosomes (LAMP-1); early endosomes (EEA1), Golgi (AP-1) and the endoplasmic reticulum (ER) (Calnexin).

As shown in FIG. 3, both MARCH-I and MARCH-II displayed an intracellular, vesicular staining pattern. This staining pattern did not perfectly overlap with any of the subcellular markers, however, frequent co-staining was observed with lysosomal markers. Only minimal colocalization with the endoplasmic reticulum was observed. Additionally, both MARCH-I and MARCH-II co-fractionate with subcellular membranes (data not shown). Therefore, these proteins localize to the membranes of vesicular post-ER compartments. The staining observed is similar to that for the myxoma M153R-protein (unpublished observations), but distinct from the ER-staining observed for the gamma-2 herpesvirus proteins (Coscoy and Ganem, 2001, supra; Paulson et al., 2001, supra).

The central hydrophobic core in MARCH-proteins implied that they localize to membranous compartments. To determine the subcellular localization for some members of this family, cDNA clones of MARCH-I, II, III, IV, V and VIII were obtained and the predicted open reading frame were inserted into a mammalian expression vector. A truncated version of MARCH-IV was expressed that lacks the amino-terminal 63 amino-acids for reasons detailed herein. To facilitate detection of the encoded proteins, a FLAG-epitope was added in frame to the carboxy-terminus of each MARCH-protein. It was previously shown that carboxy-terminal epitope tagging does not interfere with the function of K3-family proteins (Coscoy and Ganem, 2000, supra; Ishido et al., 2000a, supra; Paulson et al., 2001, supra).

FLAG-tagged MARCH-proteins were expressed in HeLa cells using transient transfection. Transfectants were permeabilized and decorated with antibodies to the FLAG epitope. In addition, co-stained was performed with antibodies to various markers of subcellular compartments: lysosomes, early endosomes, Golgi and the endoplasmic reticulum (ER). FIG. 8 shows results where complete or partial co-staining was observed between one of the marker proteins and the respective MARCH protein. MARCH-I, MARCH-III, MARCH-VIII showed a distinct punctuate staining pattern. These vesicular structures partially overlapped with some of the endocytic or lysosomal vesicles. However, most vesicles did not co-stain with any of the markers tested. MARCH-II showed an ER-like staining pattern that overlapped with calnexin staining (staining a). In some cells however, MARCH-II showed a vesicular staining that did not overlap with calnexin but co-stained partially with Lamp (staining b). MARCH-IV co-stained very clearly with the Golgi-marker, whereas MARCH-V showed a reticular ER-like staining that partially overlapped with calnexin staining. The staining pattern observed for most of the MARCH proteins is thus quite distinct from the ER-staining observed for the gamma-2 herpesvirus and poxvirus proteins (Coscoy et al., 2001, supra; Paulson et al., 2001, supra). However, evidence exists that KSHV-K3 exits the ER (Hewitt et al., 2002, supra; Means, R. E., S. Ishido, X. Alvarez and J. U. Jung, EMBO J. 21:1638-1649, 2002) and we also observed a vesicular staining pattern of MV-M153R upon expression from vaccinia-virus vectors (Mansouri et al., 2003, supra). MARCH proteins are associated with subcellular membranes, most likely as transmembrane proteins. However, several of the MARCH-proteins displayed distinct steady-state distributions.

### EXAMPLE 3

### (MARCH Proteins Functioned to Downregulate Transmembrane Glycoproteins)

Cells were transfected with appropriate plasmids as well as GFP to track transfection efficiency. At 16-48 hours post-transfection, cells were removed from tissue culture dishes with 0.05% Trypsin-EDTA (Invitrogen), washed twice with ice-cold PBS, and incubated with either anti-transferrin receptor antibody (1:100 dilution from Roche), or anti-FastL antibody (1:100 dilution from Imgenex) for 30 minutes at 4° C. The cells were then washed three times with ice-cold PBS, and incubated with a PE-conjugated anti-mouse F (a,b)₂ fragment (1:100 dilution from DAKO). Cells were washed three-times in ice-cold PBS and resuspended in 2% paraformaldehyde for analysis with a BD Bioscience (San Jose, Calif.) FACScalibur flow cytometer.

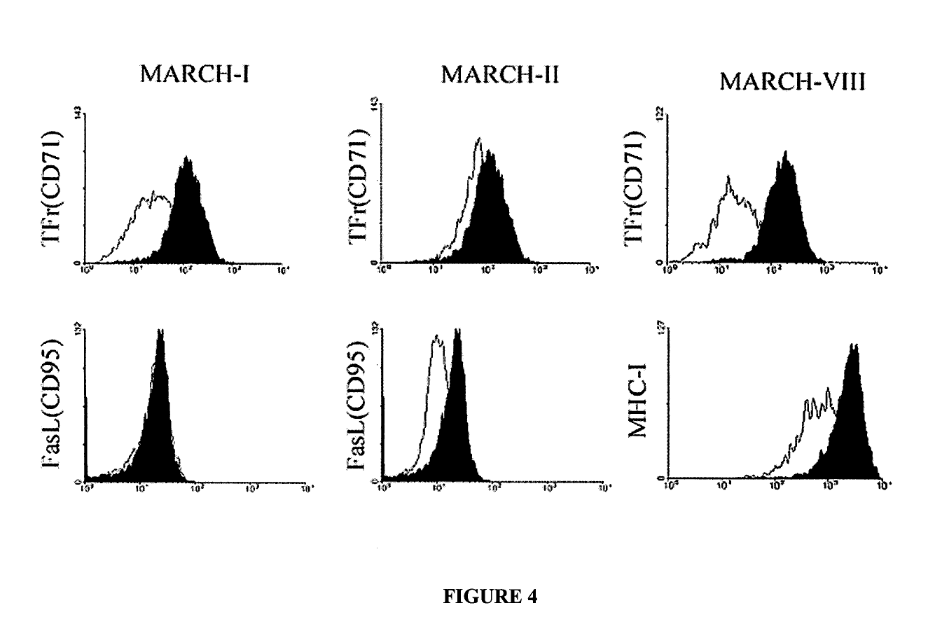

Transfection of the viral K3-family preotiens into Hela cells results in downregulation of MHC I from the cell surface. MARCH I and MARCH-II were analyzed by cytofluorometry for their ability to downregulate MHC I. No significant MHC I downregulation was observed. By contrast, as shown in FIG. 4, transfection of MARCH-VIII reduced the surface expression of MHC I. Additional surface glycoproteins were analyzed for their expression levels in the presence of MARCH-I and MARCH-II. As shown in FIG. 4, MARCH-I reduced the surface levels of transferring receptor, a type II surface glycoprotein, whereas MARCH-II reduced surface levels of CD95 (APO-1; FAS), or Pas-ligand, a type I glycoprotein. Therefore, like their viral counterparts, MARCH-proteins regulate the surface levels of transmembrance proteins. Both MARCH-I AND MARCH-II, as well as MARCH-VIII, perform a similar function in cells compared to viral proteins.

### EXAMPLE 4

### (MARCH-I and MARCH-II Formed High Molecular Weight Complexes)

HeLa cells were grown to 80% confluency in 100 mm tissue culture dishes and transfected as above. At 24 hours post-infection, cells were incubated in serum-free and methionine-free medium for 30 minutes, and metabolically labeled with ³⁵S-cysteine/³⁵S-methionine (300 μCi/plate, Amersham) for 2 hr. After labeling, cells were washed twice with PBS and lysed immediately in PBS containing 1% Digitonin and protease inhibitors (Roche, Indianapolis, Ind.). The cell lysate was precleared with protein A/G agarose beads overnight and incubated with 3 μg of antibody for one hour, followed by one hour with protein A/G beads. Immunoprecipated proteins were washed five times with 0.25% Digitonin in PBS. All samples were boiled in Laemmli buffer and analyzed by SDS-PAGE gel electrophoresis. Gels were fixed, dried and exposed to Kodak BioMax MR film. Western blotting was accomplished using the WesternBreeze Chemiluminescent Detection System (Invitrogen), following semi-dry transfer to PVDF membranes (Millipore, Bedford, Mass.).

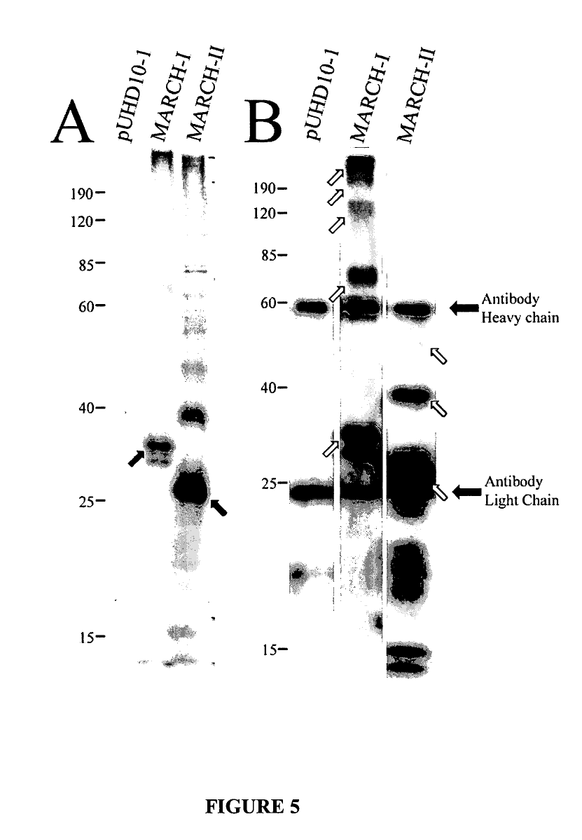

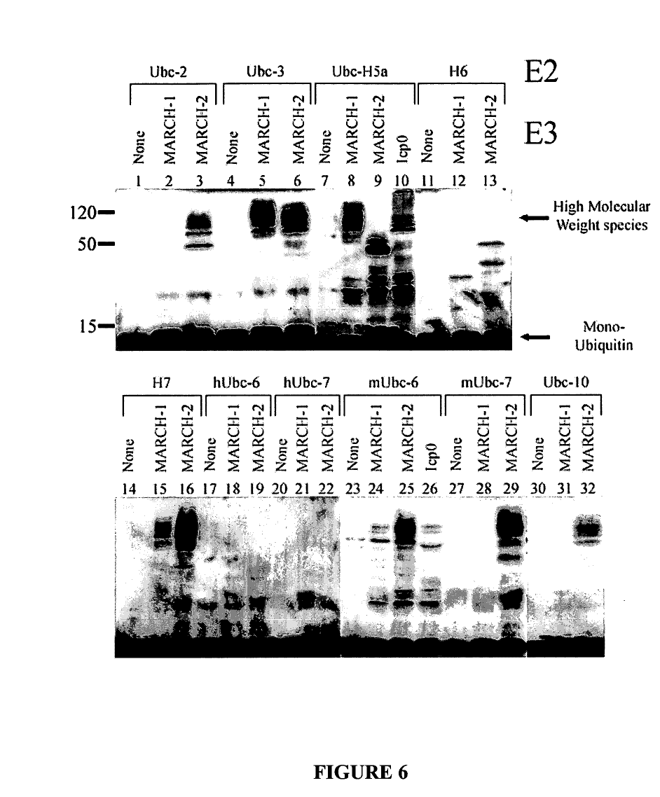

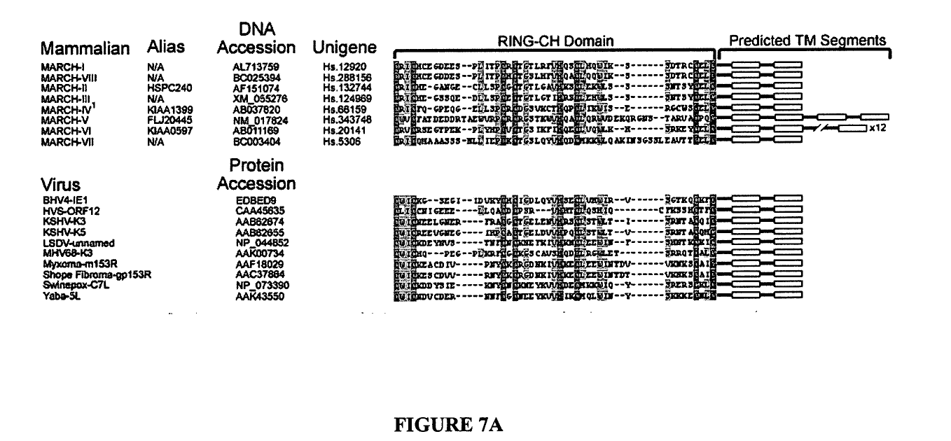

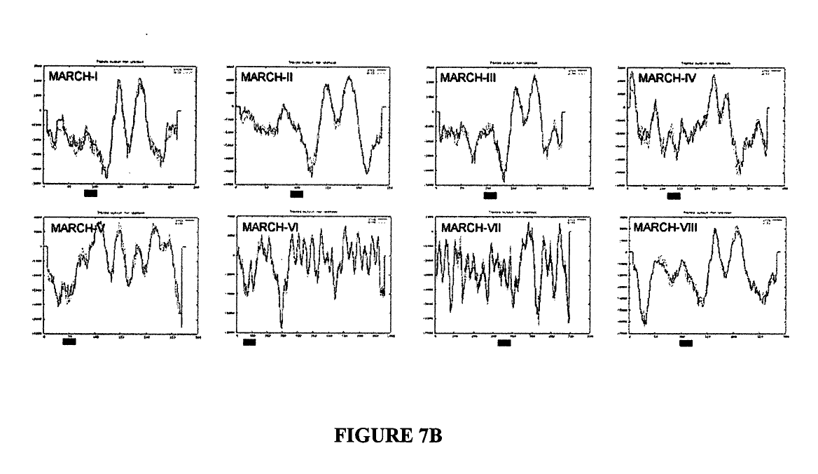

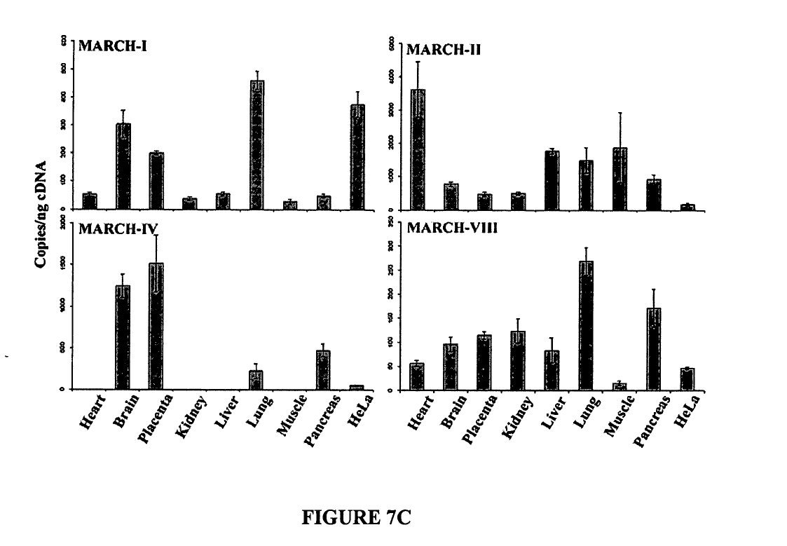

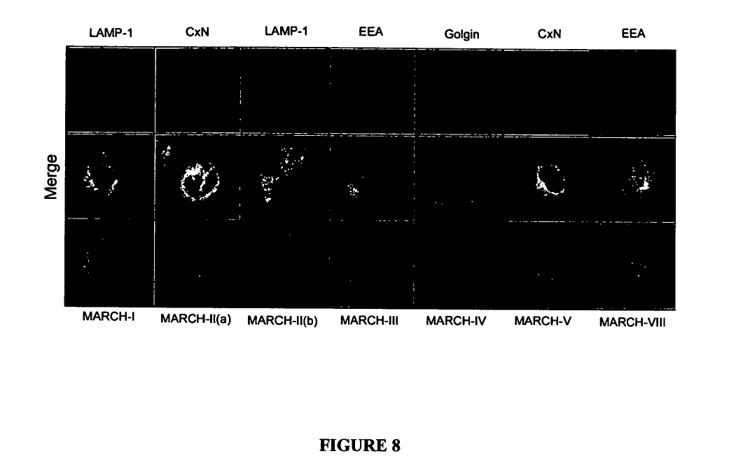

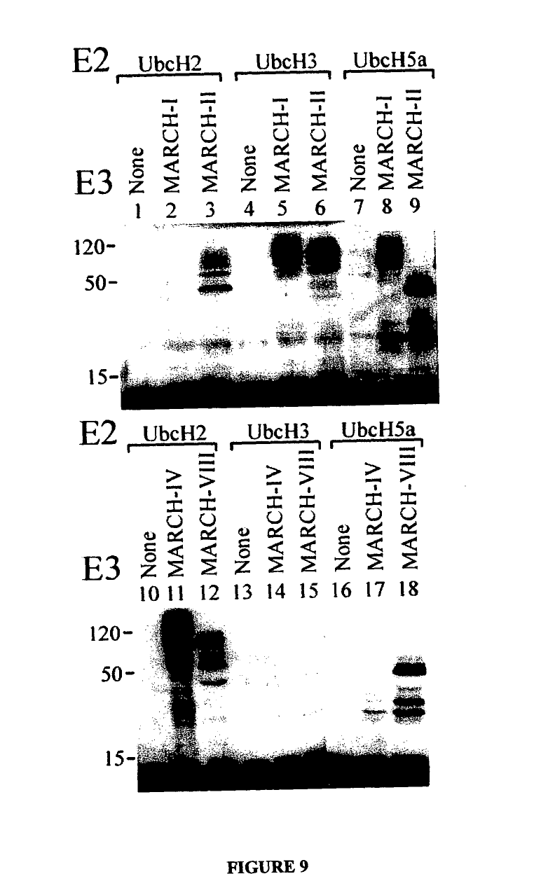

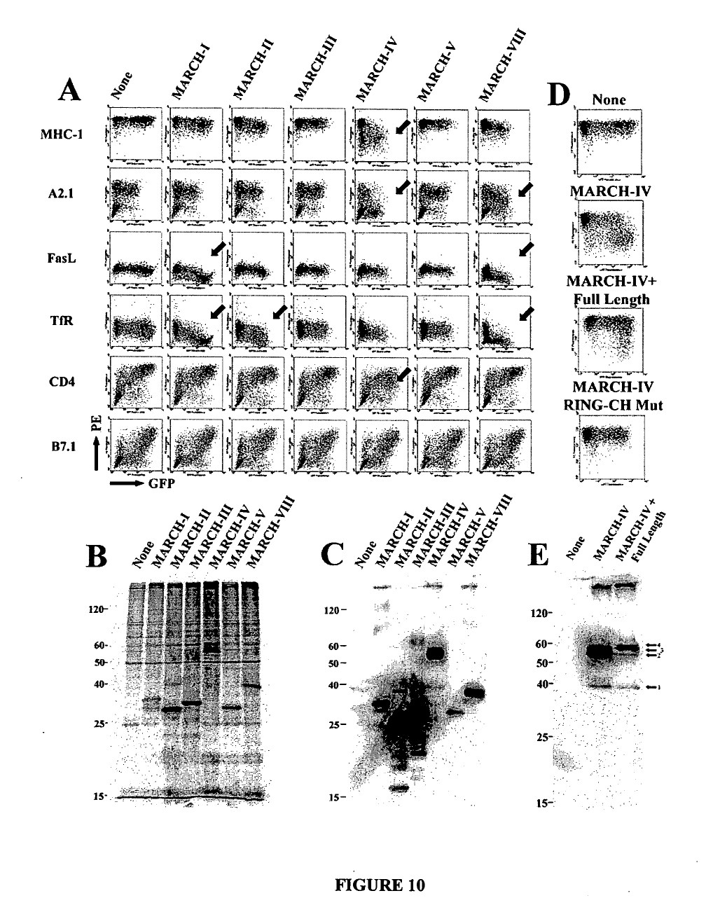

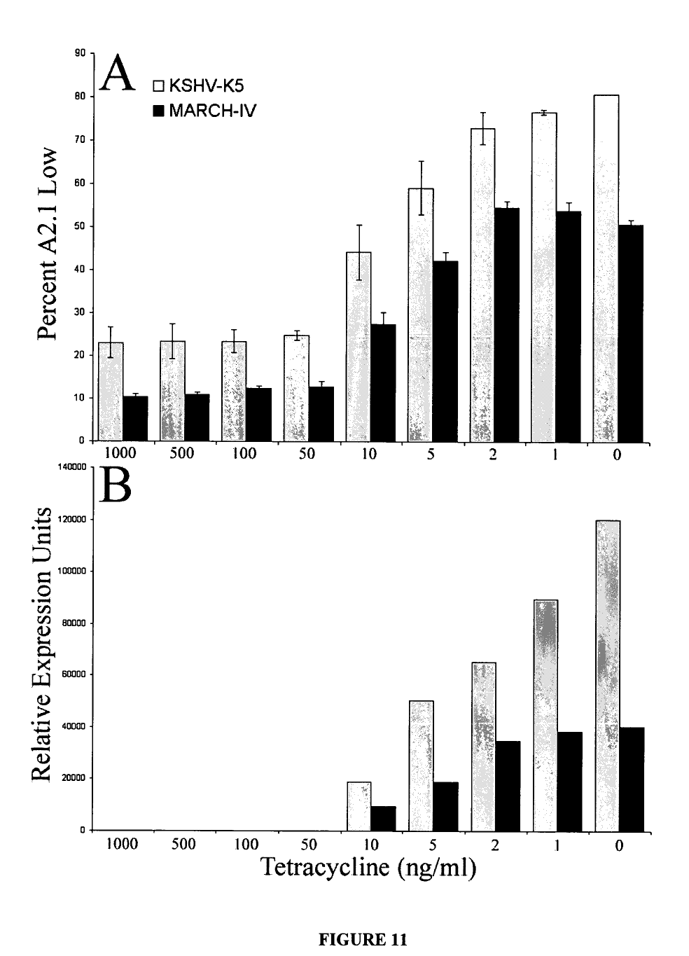

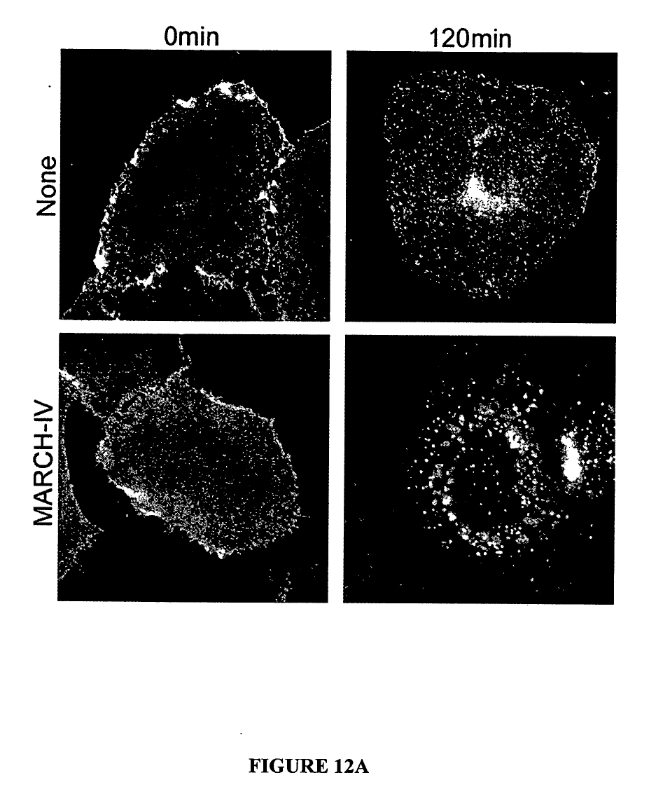

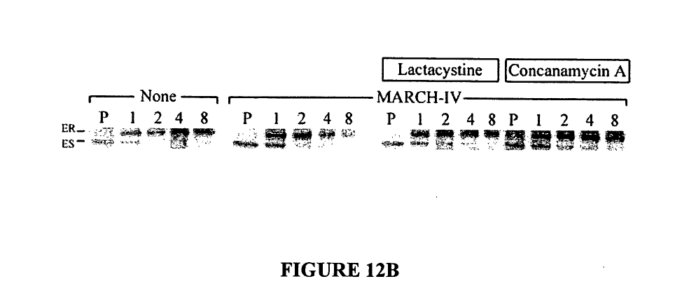

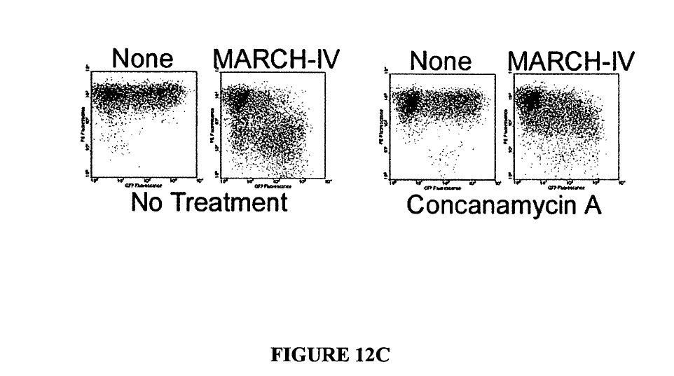

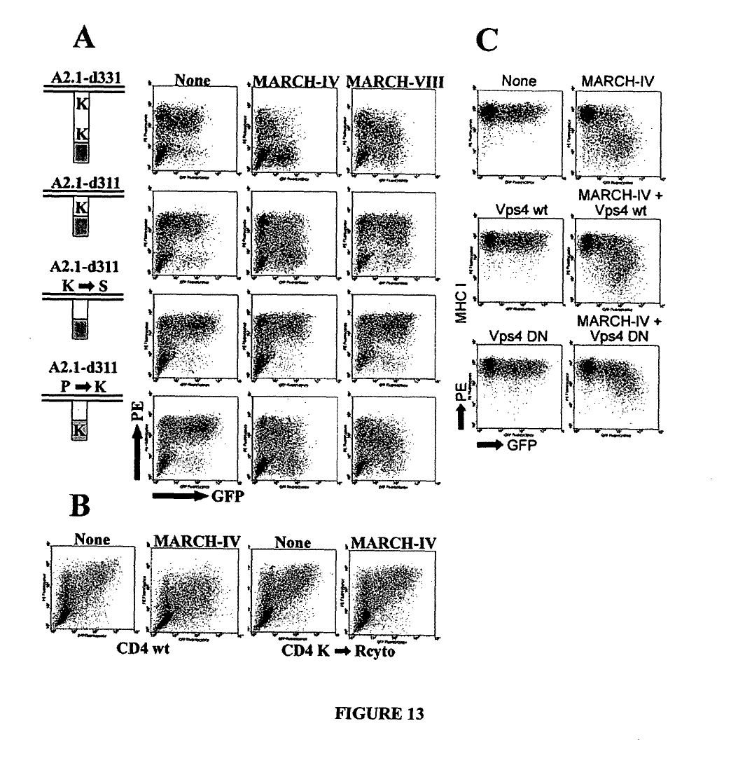

As shown in FIGS. 5 and 6, several of the high-molecular weight bands reacted with the FLAG-antibody suggesting that they represent SDS-stable protein complexes containing MARCH-I or MARCH-II. Lower molecular weight bands were also visible in MARCH-II precipitations. The small fragments are likely to be carboxy-terminal fragments since they still contain the FLAG-epitope. The higher molecular weight forms of MARCH-I match the predicted size of dimers and higher multimers. In addition, MARCH-I formed very high molecular weight complexes that were not separated in SDS-PAGE suggesting that most of MARCH-I exists in a multimeric complex. The viral protein KSHV-K5 forms similar high molecular weight complexes [Sanchez, 2001 #361] suggesting that multimer formation is one of the characteristics of this gene family. The higher molecular weight complexes of MARCH-II could also be multimeric forms, however, their molecular weight does not match the predicted size of MARCH-II dimmers and trimers. Therefore, these higher molecular weight complexes may be associations of the smaller forms with the full-length molecule. Alternatively, these higher molecular weight forms could contain a cellular protein. A potential candidate for a cellular protein was ubiquitin, however, this was not confirmed in either re-immunprecipitation or western blot experiments (data not shown).

### EXAMPLE 5

### (MARCH-I and MARCH-II were Demonstrated to have Ubiquitin-Ligase Activity)

UbCH2, (His)₆-UbCH3, GST-UbCH5a, (His)₆-UbCH6, and (His)₆-UbcH10 were obtained from Boston Biochem. GST-MmUbc6 and GST-MmUbc7 were kind gifts of A. Weissman. GST-ICP0-RING (amino acids 1-241 of ICP0) was generously provided by R. Everett, Glasgow. GST-fusion proteins were expressed in BL21 cells (Amersham), and purified as described herein.

His₆-tagged constructs of human ubc6 and ubc7 were expressed in BL21 DE3 cells, induced with 0.4 mM IPTG at OD₆₀₀ of 0.6 for 3 hours. Cells were resuspended in PBS with 0.1% NP-40, incubated for 20 minutes on ice with lysozyme and then sonicated. The soluable fraction was loaded into Ni-NTA beads (Qiagen), washed with PBS and 20 mM Imidazole. Protein was buffer exchanged, and cluted with 50 mM Tris pH 7.5, 50 mM NaCl, 10% glycerol, 0.5 β-mercapoethanol, and 200 mM Imidazole. Purified E3 proteins were stored at 4° C. and E2 proteins were stored at −80° C.

In vitro reaction mixtures (20 μl total) included: 50 nM rabbit E1 (Boston Biochem), 28 μM Ubiquitin, 10 mM ATP, 50 mM Tris-HCl pH 7.5, 50 mM NaCl, and 2 mM DTT. E2 enzymes were used at concentrations varying from 0.7 to 2.3 μM, and E3 concentrations ranged from 3 to 25 μM. After 90 minutes of incubation at 30° C., reactions were stopped by addition of 4×reducing sample buffer. Samples were electrophoresed through 12% SDS gels and blotted using anti-ubiquitin mouse antibody (Santa Cruz Biotech).

The most highly conserved feature of MARCH-I and II is the amino-terminal RING-CH domain. The related RING-H and RING-H2 domains are found in many ubiquitin ligases or E3-enzymes (Joazeiro & Weissman, Cell 102:549-552, 2000), where the RING-domain interacts with the ubiquitin-conjugating enzyme, or E2. The addition of purified RING-E3s to ubiquitin-activating enzymes (E1), E2-enzyme, and ubiquitin promotes the formation of high-molecular weight ubiquitin complexes in vitro (Joazeiro et al., Science 286:309-312, 1999). In many cases this in vitro function can be mediated by a protein fragment containing only the RING-domain. For example, the purified RING-domains of both the herpes simplex immediate early transactivator ICP0, and the RING-CH-domain-containing fragment of KSHV-K5 display ubiquitin ligase activity in vitro (Hagglund et al., Proc. Natl'l. Acad. Sci. USA 99:631-636, 2002; Boutell et al., 2002, supra; Coscoy & Ganem, 2001, supra).

To examine if the RING-CH domains of MARCH-I and MARCH-II would act as ubiquitin ligases in vitro, DNA fragments encoding the amino-terminal RING-CH-domain of both proteins were fused to Glutathione-S-transferase. The resulting constructs were sequence confirmed and expression of GST-MARCH protein verified by immunoblot. The GST-fusion protein was expressed and purified from E. coli cultures using Glutathione-columns. The ICP0-RING-domain was also purified, in parallel with the MARCH-I and MARCH-II constructs. A panel of E2 enzymes was either purchased from commercial sources or purified either by nickel-chelate chromatography or on glutathione columns. All E2-enzymes formed mercapto-ethanol-sensisitve complexes with ubiquitin thus showing ubiquitin conjugating activity. Purified ubiquitin and E1 was obtained from commercial sources.

The different E2 enzymes were co-incubated with ubiquitin, ATP, and E1 with or without RING-domain-GST fusions. As shown in FIG. 6, ICPO promoted the formation of high-molecular weight complexes in the presence of the E2 enzyme ubCH5a as observed previously (Hagglund et al., 2002, supra; Boutell et al., 2002, supra). The ICP0-RING domain also showed some activity with the murine ER-bound E2 enzyme MmUbc6 (Tiwari and Weissman, 2001, supra). MARCH-II was able to promote high molecular weight ubiquitinated complexes with several of the E2 enzymes tested. Particularly strong reactions were observed for Ubc-3, UbCH7, MmUbc6 and MmUbc7. By comparison, MARCH-I was less active with most E2 enzymes tested with the notable exception of UbCH5a. These anti-ubiquitin reactive high molecular weight complexes were not observed in the absence of E3-enzymes, as shown in FIG. 6, or in the presence of purified GST (not shown). Therefore, MARCH-I and MARCH-II proteins were determined to function as ubiquitin ligases.

### EXAMPLE 6

### (RING-CH Domains of MARCH Proteins were Found to have Ubiquitin-Ligase Activity)

The presence of a predicted RING-CH domain suggests that MARCH-proteins possess ubiquitin ligase activity. RING-E3s are known to catalyze the formation of ubiquitin-adducts in vitro when co-incubated with ubiquitin, ATP, E1, and an appropriate E2 (Joazeiro et al., 1999, supra). In many cases a protein fragment containing only the RING-domain can mediate this in vitro function, e.g., the RING-CH domains of KSHV-K5, MV-M153R or yeast-SSM4 (Coscoy et al., 2001, supra; Mansouri et al., 2003, supra; Swanson et al., 2001, supra).

To examine if the RING-CH domains of MARCH proteins act as ubiquitin ligases in vitro, a DNA fragment encoding the amino-terminal RING-CH-domains of MARCH-I, II, IV and VIII were fused to Glutathione-S-transferase (GST) for bacterial expression. The GST-fusion proteins were purified from E. coli cultures using Glutathione-sepharose-columns. Approximately thirty E2 proteins are found in the human genome compared to only one E1 enzyme (Pickart, 2001, supra). This implies that E3s possess a certain degree of specificity towards certain E2s. The specificity and selectivity of E3s with respect to E2s, however, is not well characterized. To cover a spectrum of E2s, a panel of E2 enzymes (Mansouri et al., 2003, supra) was acquired. The various E2 enzymes were incubated with or without the RING-CH-GST fusions in the presence of ubiquitin, ATP, E1. The formation of multiubiquitin-adducts was examined by immunoblotting with antibodies against ubiquitin.

Representative results obtained with three human E2 enzymes UbCH2, UbCH3 and UbCH5a, are shown in FIG. 9. Anti-ubiquitin reactive high molecular weight complexes were not observed in the absence of E3-enzymes (FIG. 9), or in the presence of purified GST. The RiNG-CH-GST fusion proteins of all four MARCH-proteins tested were able to catalyze the formation of high-molecular weight molecular ubiquitin complexes in the presence of at least one of these three enzymes. The yield of these complexes, however, varied depending on the combination of MARCH-protein and E2 enzyme. MARCH-II was able to promote high molecular weight ubiquitinated complexes with several of the E2 enzymes tested. In comparison, MARCH-IV was mostly active with UbCH2. These results further demonstrate that MARCH proteins act as ubiquitin ligases. Moreover, the data show that the RING-CH domain acts as an ubiquitin ligase module.

### EXAMPLE 7

### (Cell Surface Receptors were Downregulated by MARCH-Proteins)

All viral K3-family members examined to date were shown to downregulate expression of cell surface molecules. The ability to downregulate MHC I is common to KSHV-K3, KSHV-K5, MHV68-K3 and MV-M153R (Coscoy and Ganem, 2000, supra; Guerin et al., 2002, supra; Ishido et al., 2000b supra; Stevenson et al., 2000, supra). Additional substrates are ICAM-1 and B7.2 (but not B7.1) for KSHV-K5, as well as CD95 and CD4 for M153R (Coscoy and Ganem, 2001, supra; Guerin et al., 2002, supra; Ishido et al., 2000a, supra; Mansouri et al., 2003, supra). Since only low copy numbers were detected in HeLa cells for transcripts corresponding to each of the MARCH-proteins (FIG. 7C, and data not shown), HeLa cells were transfected with expression plasmids for MARCH-I, II, III, IV, V and VIII. Surface expression of selected glycoproteins was monitored upon transfection. The following known substrates for viral K3-family members were tested: Fas (CD95) and HLA class I (MHC I), both of which are endogenously expressed in HeLa cells, as well as transfected HLA-A2.1 or CD4. Endogenously expressed transferrin receptor and transfected B7.1 were used as controls, neither of which is downregulated by the viral proteins (Coscoy and Ganem, 2001, supra; Guerin et al., 2002, supra; Ishido et al., 2000a, supra; Mansouri et al., 2003, supra). Surface expression of the respective markers was analyzed by flow cytometry. GFP was used to identify transfected cells.

The results are plotted as fluorescence of the respective surface markers versus fluorescence of GFP (FIG. 10A). Interestingly, the expression of every surface glycoprotein was affected by at least one of the MARCH-proteins with the notable exception of B7.1 (discussed herein). Endogenous MHC I, detected by the pan-specific monoclonal antibody W6/32, was significantly downregulated by MARCH-IV, whereas all other MARCH-proteins had no or only marginal effects on MHC I. MARCH-IV also reduced the surface expression of transfected HLA-A2.1 and CD4. Surface levels of transfected A2.1 were also reduced by MARCH-VIII, although more striking was the almost complete elimination of transferrin receptor (TfR) surface levels by this protein. TfR-downregulation was not previously reported for any of the viral proteins and TfR is the only type II transmembrane protein included in our panel. Some reduction of TfR was also seen in MARCH-I and II transfected cells. In addition, both MARCH-I and MARCH-VIII reduced the surface expression of Fas. No reduction in surface levels was observed for any of the glycoproteins in cells transfected with MARCH-III or the four-transmembrane MARCH-V. To eliminate the possibility that this was due to a lack of expression we examined the expression of FLAG-tagged versions of each MARCH-protein by immunoprecipitation (FIG. 10B) and immunoblot (FIG. 10C). While there were differences in expression levels, all of the proteins were readily detectable in transfected cells. Since there was no clear correlation between expression levels and observed effect on surface levels of specific glycoproteins, the differential effects seen in FIG. 10A are caused by different substrate specificities of the individual MARCH-proteins. This result is reminiscent of the viral proteins K3 and K5 which share the ability to downregulate HLA-A and B alleles but differ with respect to HLA-C as well as ICAM-1 and B7-2 (Coscoy and Ganem, 2001, supra; Ishido et al., 2000a, supra).

The molecular weight of all MARCH-proteins corresponded to the predicted molecular weight with the exception of MARCH-IV. As already mentioned, the MARCH-IV construct used in these experiments lacks the first 63 amino-acids and is thus predicted to have a MW of 38 kd. The observed MW however, is closer to 55 kd. The reason for this shift in molecular weight is currently not known. However, the molecular weight is too low to represent a dimer or multimer as observed for K5 (Sanchez et al., 2001, supra). To further evaluate the molecular weight of MARCH-IV, the predicted full-length version of MARCH-IV was expressed as a FLAG-tagged construct. Immuno-precipitation followed by immunoblot revealed four distinct molecular weight forms (FIG. 10E). The molecular weight of the shortest (product 1) corresponds in to the MW of the construct lacking the first 63 amino-acids. In addition, a doublet of higher MW weight proteins (products 2 and 3) is also found in cells expressing the truncated version. Also present is a protein product 4 corresponding to the predicted molecular weight version of the full-length construct. Thus, MARCH-IV is translated into several products that share the carboxyterminus since they all react with the FLAG-antibody. The full-length version downregulates MHC I (FIG. 10D). Most likely, however, the shorter forms mediate this downregulation since they are the predominant translational product of full-length MARCH-IV. Since the shorter version more closely resembles the viral proteins, this version was used in examples 6 through 8.

To determine if a functional RING-CH domain is required for the observed downregulation of MHC I by MARCH-IV, a mutant of the MARCH-IV protein with two conserved cysteines replaced by serines was generated. Upon transfection of HeLa cells, the resulting construct was unable to reduce the surface expression of MHC I (FIG. 10D). This result indicates that the ubiquitin ligase activity is required for downregulation of surface receptors by MARCH-IV. This is consistent with reported requirement of the RING-CH domain for the viral proteins (Boname and Stevenson, 2001, supra; Ishido et al., 2000b, supra; Paulson et al., 2001, supra; Sanchez et al., 2001, supra).

With regards to whether MHC I downregulation by MARCH-IV was only observed at very high expression levels, since the viral immune evasion genes seem to be dedicated to MHC I downregulation, MHC I expression was compared at different expression levels of MARCH-IV or KSHV-K5 by taking advantage of the tetracycline-regulatable system to partially express either protein (Gossen, M. and H. Bujard, Proc. Natl. Acad. Sci. USA 89:5547-5551, 1992). Tet-off HeLa cells were transfected with either MARCH-IV or with KSHV-K5 in the presence of increasing amounts of tetracycline to shut off transcription. After 48 hours, surface expression of MHC I molecules was analyzed by flow cytometry (FIG. 11A) and expression of MARCH-IV and K5 (both FLAG-tagged) was determined by immunoblot (FIG. 11B). In both cases, partial MHC I downregulation below the detectable level of RING-CH protein expression was observed. MARCH-IV was consistently expressed at lower levels compared to KSHV-K5 which might indicate that high levels of MARCH-IV expression are not well tolerated by transfected cells. A lower percentage of MARCH-IV transfectants showed reduced MHC I expression levels compared to KSHV-K5, although this could be explained by the lower expression levels of MARCH-IV. The efficient downregulation of MHC I at low expression levels suggests an efficient MHC I downregulation by MARCH-IV comparable to KSHV-K5.

### EXAMPLE 8

### (Molecular Mechanism of MHC I Downregulation by MARCH-IV)

The results obtained above suggest that the MARCH-family is functionally as well as structurally related to the viral K3-family. The observed downregulation of MHC I and CD4 by MARCH-IV permitted the study of the molecular mechanism by which MARCH-proteins reduce glycoprotein levels. Most members of the viral K3-family reduce expression of their target substrates by inducing a rapid internalization from the cell surface. The only known exception is MHV68-K3 which mediates proteasomal degradation of MHC I prior to its exit from the ER (Boname and Stevenson, 2001, supra). To study if MHC I was internalized by MARCH-IV, the recycling of MHC I in MARCH-IV transfected and non-transfected cells over the course of 2 hours was compared. MHC I on MARCH-IV transfected or non-transfected cells was labeled with anti-MHC I antibody at 4° C. Cells were then either fixed immediately or transferred to 37° C. for 120 minutes to allow uptake of MHC I. After fixation and visualization with a secondary antibody, a distinct punctuate staining was observed in transfected cells (a representative example is shown in FIG. 12A), whereas a uniform surface staining was observed in non-transfected cells. This rapid internalization is very similar to the uptake of MHC I observed in KSHV-K5, KSHV-K3 and MV-M153R-transfected cells (Coscoy and Ganem, 2000, supra; Ishido et al., 2000b, supra; Mansouri et al., 2003, supra).

To determine the ultimate fate of the MHC I molecules in the presence of MARCH-IV, the maturation and turnover of co-transfected HLA-A2.1 in pulse-chase experiments (FIG. 12B) was followed. In the absence of MARCH-IV, HLA-A2.1 molecules acquired Endo H resistance after 1 hour of chase indicating exit from the ER. A similar kinetic of exit was observed in MARCH-IV-transfected cells. However, lower amounts of HLA-A2.1 were immunoprecipitated at later time points in MARCH-IV-transfected cells whereas HLA-amounts did not decrease in the absence of MARCH-IV. This indicates that HLA-A2.1 exits the ER but is degraded in the presence of MARCH-IV. In order to determine if this degradation occurred via the proteasomal pathway or via the endosomal/lysosomal system, MARCH-IV and HLA-A2.1 co-transfected cells with proteasomal or lysosomal inhibitors during the labeling and chase periods were treated. While the proteasome inhibitor lactacystin did not increase recovery of HLA-A2.1 heavy chains compared to untreated cells, inhibition of endosomal acidification by concanamycin A delayed the degradation of HLA-A2.1. Concanamycin A has no effect in the absence of MARCH-IV. Overnight treatment with concanamycin A also restored surface expression of MHC I in MARCH-IV transfected cells (FIG. 12C). These results are consistent with HLA-A2.1 exiting the ER, being transported to the cell surface and then rapidly internalized by endocytosis for delivery to the lysosome.

The proposed function of this protein family as ubiquitin ligases implies a role for ubiquitination in the internalization process. Ubiquitinated cell surface receptors are internalized and sorted to lysosomes via MVBs, a process that is regulated by several sequential multiprotein ESCRT complexes (Babst, M., D. J. Katzmann, E. J. Estepa-Sabal, T. Meerloo and S. D. Emr, Dev. Cell 3:271-282, 2002a; Babst, M., D. J. Katzmann, W. B. Snyder, B. Wendland, and S. D. Emr, Dev. Cell 3:283-289, 2002b; Katzmann, D. J. M. Babst and S. D. Emr, Cell 106:145-155, 2001). After completion of the MVB sorting, the AAA-type ATPase vps 4 (vacuolar protein sorting protein 4) is required for the dissociation of the ESCRT complex from the membrane (Babst et al., 2002a, supra). Using a mutant form of vps4 that lacks the ATP-binding domain (Garrus, J. E., U. K. von Schwedler, O. W. Pornillos, S. G. Morham, K. H. Zavitz, H. E. Wang, D. A Wettstein, K. M. Stray, M. Cote and R. L. Rich, Cell 107:55-65, 2001) whether, the internalization of MHC I in MARCH-IV-transfected cells involves MVB formation was examined. Surface expression of MHC I was monitored on HeLa cells, co-transfected with MARCH-IV and either wildtype or mutant vps 4. Neither mutant nor wildtype vps4 changed the surface levels of MHC I in HeLa cells in the absence of MARCH-IV. In contrast, co-transfection of mutant vps4 with MARCH-IV restored surface MHC-I expression (FIG. 13C). MARCH-IV-mediated MHC I internalization, therefore, involves the MVB pathway.

The most likely targets for ubiquitination on transmembrane glycoproteins are lysines in the cytoplasmic tail. To examine the role of lysines for MHC I and CD4 downregulation by MARCH-IV, a panel of constructs derived from the human MHC I molecule HLA-A2.1 and a lysine-deleted version of CD4 (Mansouri et al., 2003, supra) were used. In addition to MARCH-IV, MARCH-VIII was included in this analysis since it reduced HLA-A2.1 surface expression (FIG. 10A). Flow cytometry revealed that removal of lysines from the tail of HLA-A2.1 or CD4 renders these molecules resistant to MARCH-IV or MARCH-VIII downregulation (FIGS. 13A and 13B). To rule out the possibility that these mutations changed the target molecules in such a way that they are no longer recognized by MARCH-IV or VIII, a lysine residue into a HA-tag at the carboxyterminal end of HLA-A2.1 was introduced. This re-introduction of a lysine residue into a sequence not related to HLA-A2.1 restored downregulation both by MARCH-IV and by MARCH-VIII.

Therefore, lysines in the tail are an absolute requirement for MARCH-IV and MARCH-VIII. These results are consistent with reports that glycoprotein targets that lack lysines in their cytosolic tail are resistant to downregulation by the viral K3-family (Boname and Stevenson, 2001, supra; Coscoy et al., 2001, supra; Mansouri et al., 2003, supra). Moreover, transfer of the lysine-containing tail of B7.2 onto B7.1 confers susceptibility to K5-mediated internalization (Coscoy et al., 2001, supra). The lack of lysines in the tail of B7.1 is thus the most likely reason why this molecule was the only one not affected by any of the MARCH proteins (FIG. 10A). Taken together, the MARCH-proteins represent a family of transmembrane ubiquitin ligases that target lysines in the tail of their substrates for ubiquitination and subsequent internalization.

No license is expressly or implicitly granted to any patent or patent applications referred to or incorporated herein. The discussion above is descriptive, illustrative and exemplary and is not to be taken as limiting the scope defined by any appended claims.

1. A method for treating or preventing a condition affected by endocytosis or down-regulation of cell surface receptors, comprising administering to a patient in need thereof a therapeutically effective amount of a MARCH-protein antagonist or pharmaceutical composition thereof, wherein the antagonist inhibits MARCH-protein-mediated endocytosis or down-regulation of type I or type II cell surface receptors.

2. The method of claim 1, wherein the MARCH antagonist is selected from the group consisting of anti-MARCH-protein antibodies or eptitope-binding fragments thereof, MARCH-specific antisense oligomers, proteins and polypeptides, small molecule MARCH inhibitors of MARCH-protein-mediated ubiquitination, and combinations thereof.

3. The method of claim, 2, wherein the MARCH-specific antisense oligonucloetide comprises a sequences of at least 12 contiguous nucleotides of a sequence selected from the group consisting of SEQ ID NOS:38, 40, 42, 44, 46, 48, 50 and SEQ ID NO:52, and complements thereof

4. The method of claim 1, wherein the MARCH-protein is selected from the group consisting of MARCH-I through MARCH-VII, and MARCH-VIII, corresponding to SEQ ID NOS:39 and 54, SEQ ID NOS:41 and 55, SEQ ID NOS:43 and 56, SEQ ID NOS:45 and 57, SEQ ID NOS:47 and 58, SEQ ID NOS:49 and 59, SEQ ID NOS:51 and 60, SEQ ID NOS:53 and 61, respectively.

5. The method of claim 1, wherein the MARCH-protein-mediated endocytosis of type I or type II cell surface receptors, involves a receptor selected from the group consisting of transferrin receptor, FAS (APO-1/CD95), MHC-I and other histocompatibility antigens, HLA-A2.1 or CD4, ICAM-1, B7.2, and combinations thereof.

6. The method of claim 1, wherein the condition is selected from the group consisting of leukemia and other cancers, mental retardation, thalessemia, autoimmune disease and neurological disease.

7. A method for identifying test compounds having therapeutic activity for a condition affected by endocytosis of cell surface receptors, comprising:
(a) contacting a test compound with a MARCH-protein target/receptor and a functional MARCH-protein wherein at least one of the proteins bears a detectable label;
(b) assaying any resulting MARCH-protein target/receptor:MARCH-protein complex for the presence of the label; and
(c) determining whether the test compound antagonizes binding of the MARCH-protein target/receptor to the MARCH-protein, whereby test compounds that antagonize said binding are, at least in part, identified as therapeutic compounds.

8. The method of claim 7, wherein the MARCH-protein is selected from the group consisting of MARCH-I through MARCH-VII, and MARCH-VIII, corresponding to SEQ ID NOS:39 and 54, SEQ ID NOS:41 and 55, SEQ ID NOS:43 and 56, SEQ ID NOS:45 and 57, SEQ ID NOS:47 and 58, SEQ ID NOS:49 and 59, SEQ ID NOS:51 and 60, SEQ ID NOS:53 and 61, respectively.

9. The method of claim 7, wherein the MARCH-protein target/receptor is selected from the group consisting of transferrin receptor, FAS (APO-1/CD95), MHC-I and other histocompatibility antigens, HLA-A2.1 or CD4, ICAM-1, B7.2, and combinations thereof.

10. The method according to claim 7 wherein either the functional MARCH-protein target/receptor or the functional MARCH-protein is immobilized onto a solid phase.

11. The method according to claim 7 wherein the MARCH-protein target/receptor or the MARCH-protein is labeled with a radiolabel, a fluorescent reporter or quencher moiety, an enzymic label that catalyzes a colorometric or fluorometric change, or combinations thereof.

12. A method for identifying test compounds having therapeutic activity for a condition affected by endocytosis of cell surface receptors, comprising:
(a) contacting a test compound with a cell expressing a functional MARCH-protein target/receptor and a MARCH-protein; and
(b) determining whether the test compound antagonizes at least one of MARCH-protein-mediated ubiquination activity, or MARCH-protein-mediated receptor endocytosis, whereby test compounds that antagonize at least one of such activity are identified as therapeutic compounds.

13. The method of claim 12 wherein the cell expresses a recombinant MARCH-protein target/receptor or a recombinant MARCH-protein.

14. The method of claim 12, wherein determination of antagonism of MARCH-protein-mediated activity is based on an assay selected from the group consisting of: inhibition of MARCH-protein-mediated endocytosis assays, receptor up- or down-regulation assays, cell motility assays, cell growth rate assays, apoptosis assays, ubiquination assays, and MARCH-protein-mediated or MARCH-protein target/receptor-mediated signal transduction assays.

15. The method of claim 14 wherein the MARCH-protein-mediated endocytosis assay is based on measurement of a cell-surface receptor selected from the group consisting of transferrin receptor, FAS (APO-1/CD95), MHC-I and other histocompatibility antigens, HLA-A2.1 or CD4, ICAM-1, B7.2, and combinations thereof.

16. The method of claim 14 wherein the MARCH-protein-mediated or MARCH-protein target/receptor-mediated signal transduction assay is based on measurement of cellular processes selected from the group consisting of phosphorylation or activation of an intracellular protein, organization of the actin cytoskeleton, gene transcription, lipid metabolism, vesicle trafficking, cellular transformation, cellular death, and combinations thereof, whereby test compounds that alter said cellular process, relative to those of control cells, are identified as therapeutic compounds.

17. The method according to claim 14, wherein the condition characterized by MARCH-protein-mediated cell growth, cell motility, or inhibition of endocytosis is cancer, mental retardation, or thalessemia.

18. A pharmaceutical composition, comprising an antagonist of a MARCH-protein, and a pharmaceutically acceptable carrier or diluant.

19. The pharmaceutical composition of claim 18, wherein the MARCH-protein antagonist is a MARCH-specific antisense oligonucleotide comprising at least 12 contiguous nucleotides of a sequence selected from the group consisting of of SEQ ID NOS:38-45, and complements thereof.

20. An anti-MARCH-protein antibody.

21. The anti-MARCH-protein antibody of claim 20, wherein the antibody is a monoclonal antibody.

22. The anti-MARCH-protein monoclonal antibody of claim 21, wherein the monoclonal antibody is a single-chain antibody, chimeric antibody, humanized antibody or Fab fragment.

23. A method for inhibiting endocytosis or down-regulation of cell-suface receptors, comprising contacting a cell, having a cell-surface receptor that is at least to some extent subject to MARCH-protein-meidated endocytosis, with a MARCH-protein antagonist, whereby said endocytosis or down-regulation is, at least to some extent, inhibited.

24. The method of claim 23, wherein the MARCH antagonist is selected from the group consisting of anti-MARCH-protein antibodies or eptitope-binding fragments thereof, MARCH-specific antisense oligomers, proteins and polypeptides, small molecule MARCH inhibitors of MARCH-protein-mediated ubiquitination, and combinations thereof.

25. The method of claim, 24, wherein the MARCH-specific antisense oligonucloetide comprises a sequences of at least 12 contiguous nucleotides of a sequence selected from the group consisting of SEQ ID NOS:38, 40, 42, 44, 46, 48, 50 and SEQ ID NO:52, and complements thereof

26. The method of claim 23, wherein the MARCH-protein is selected from the group consisting of MARCH-I through MARCH-VII, and MARCH-VIII, corresponding to SEQ ID NOS:39 and 54, SEQ ID NOS:41 and 55, SEQ ID NOS:43 and 56, SEQ ID NOS:45 and 57, SEQ ID NOS:47 and 58, SEQ ID NOS:49 and 59, SEQ ID NOS:51 and 60, SEQ ID NOS:53 and 61, respectively.

27. The method of claim 23, wherein the MARCH-protein-mediated endocytosis or down-regulation of type I or type II cell surface receptors, involves a receptor selected from the group consisting of transferrin receptor, FAS (APO-1/CD95), MHC-I and other histocompatibility antigens, HLA-A2.1 or CD4, ICAM-1, B7.2, and combinations thereof.


SEQUENCE LISTING (66 sequences)

SEQ ID NO: 1 - DNA, 29 bp, Homo sapiens
cctagaattcaccatgaccagcagccacg

SEQ ID NO: 2 - DNA, 28 bp, Homo sapiens
atcgggatcctcagactgatacaacttc

SEQ ID NO: 3 - DNA, 51 bp, Homo sapiens
atcgggatcctcacttgtcatcgtcatccttgtagtcgactgatacaactt

SEQ ID NO: 4 - DNA, 31 bp, Homo sapiens
atcggcggccgctcatttcctcctttcactt

SEQ ID NO: 5 - DNA, 28 bp, Homo sapiens
cctagaattcaccatgacgacgggtgac

SEQ ID NO: 6 - DNA, 31 bp, Homo sapiens
atcgggatcctcatactggtgtctcctctgc

SEQ ID NO: 7 - DNA, 55 bp, Homo sapiens
atcgggatcctcacttgtcatcgtcatccttgtagtctactggtgtctcctctgc

SEQ ID NO: 8 - DNA, 32 bp, Homo sapiens
atcggcggccgctcatgtccgcttctccgtcc

SEQ ID NO: 9 - DNA, 28 bp, Homo sapiens
atcggaattcaccatgacaaccagccgc

SEQ ID NO: 10 - DNA, 27 bp, Homo sapiens
atcgggatcctcaaacaactgtctcct

SEQ ID NO: 11 - DNA, 51 bp, Homo sapiens
atcgggatcctcacttgtcatcgtcatccttgtagtcaacaactgtctcct

SEQ ID NO: 12 - DNA, 29 bp, Homo sapiens
atcggaattcaccatgcacggcgaccccc

SEQ ID NO: 13 - DNA, 33 bp, Homo sapiens
atcggaattcaccatgctgatgcccctgtgtgg

SEQ ID NO: 14 - DNA, 27 bp, Homo sapiens
atcgggtacctcacactgtcgtgactc

SEQ ID NO: 15 - DNA, 51 bp, Homo sapiens
atcgggtacctcacttgtcatcgtcatccttgtagtccactgtcgtgactc

SEQ ID NO: 16 - DNA, 36 bp, Homo sapiens
tcgatacgcggccgctcactgaaccttctcaatgac

SEQ ID NO: 17 - DNA, 24 bp, Homo sapiens
ggatccaccatgcacggcgacccc

SEQ ID NO: 18 - DNA, 34 bp, Homo sapiens
cgcgtctagagcgtcatcacactgtcgtgactct

SEQ ID NO: 19 - DNA, 28 bp, Homo sapiens
atcgggtaccaccatgccggaccaagcc

SEQ ID NO: 20 - DNA, 29 bp, Homo sapiens
atcgggatcctcatgcttcttcttgttct

SEQ ID NO: 21 - DNA, 53 bp, Homo sapiens
atcgggatcctcacttgtcatcgtcatccttgtagtctgcttcttcttgttct

SEQ ID NO: 22 - DNA, 29 bp, Homo sapiens
atcggaattcaccatgagcatgccactgc

SEQ ID NO: 23 - DNA, 28 bp, Homo sapiens
atcgggtacctcagacgtgaatgatttc

SEQ ID NO: 24 - DNA, 52 bp, Homo sapiens
atcgggtacctcacttgtcatcgtcatccttgtagtcgacgtgaatgatttc

SEQ ID NO: 25 - DNA, 44 bp, Homo sapiens
tcgatacgcggccgctcacttcctgcgctcgctggacgtcatct

SEQ ID NO: 26 - DNA, 21 bp, artificial sequence
  MARCH-I Forward Primer
ccacaaacaggtgcaaattca

SEQ ID NO: 27 - DNA, 21 bp, artificial sequence
  MARCH-I Reverse primer 2
tcccaacaggttccatcagac

SEQ ID NO: 28 - DNA, 20 bp, artificial sequence
  MARCH-2 Forward primer
gctgcgacatggtgtgtttc

SEQ ID NO: 29 - DNA, 20 bp, artificial sequence
  MARCH-2 Reverse primer
aggcacaaccagcctgagat

SEQ ID NO: 30 - DNA, 21 bp, artificial sequence
  MARCH-IV Forward primer 2
tggctcatctggtcaactttc

SEQ ID NO: 31 - DNA, 21 bp, artificial sequence
  MARCH-IV Reverse primer 2
tgtcttgtcatagttcagcac

SEQ ID NO: 32 - DNA, 19 bp, artificial sequence
  MARCH-VIII Forward primer 2
tcctccgtcagcatcagct

SEQ ID NO: 33 - DNA, 19 bp, artificial sequence
  MARCH-VIII Reverse primer 2
ggctggatggcgtgataga

SEQ ID NO: 34 - DNA, 21 bp, artificial sequence
  MARCH-I orf forward primer
agaatctgtcactgcgaaggg

SEQ ID NO: 35 - DNA, 19 bp, artificial sequence
  MARCH-I orf reverse primer
agtgtcccagtgcagcgac

SEQ ID NO: 36 - DNA, 20 bp, artificial sequence
  MARCH-II orf forward primer
gctgcgacatggtgtgtttc

SEQ ID NO: 37 - DNA, 20 bp, artificial sequence
  MARCH-II orf reverse primer
aggcacaaccagcctgagat

SEQ ID NO: 38 - DNA, 2715 bp, Homo sapiens
  CDS (978)..(1847)
agaggaggggagaggactggggagccgagccagagccgggctgcctgccacccggctgctcgtccgctagctggggaggagcgctccacccgcaactgacaaaggatgggagaatgcccgcgccccgggatgccggccgcacgcagcctggcggccgcctgagctacttcaccctccgccggtaagtgactgcaaacatcattcattcaatcagcctcactgggagccccttctctccggctggtagtcctgggcggcttgtccctgatcccgagcggggcttggcacagcatcagccctggagggcaggcagcaggtgcctttgcctggtgggtccactggggagcgtggctggggttcgcggcgggtgctgccacccaacctgcgggcggcgggctcgcccagtaggcgcctctctggtgagaggaggcggctccagcccgcatcctggggtagttgctactattggcccccagcgcccgctctgcgcgcgcgccgtttctggcggatccccagtgcgcggcgcgctgtttacaccggcgtggtactagtcacggagccgcacccctcggaaagcgcggagtcgatgacagccacttcacaggctcacgcgctcctagtgtgggcttgaaggggacggggaccgattaccaaaggagagcgctgagtacggaagacacagggcagcctttgtcttgggtttagcgctgatgcgctcaaccctgagtcgggttcactgcaactgttgtgtccgatttcggttccctgcaaccgccctcctgggcgagagatgtcattgtgttcctgcggccagcgggactgagagctgggacttaagacgccaggagggtcctgcgctcacgggaaatgtaccccaaaagaactctgagagaatatactcaactgtcctgctgtgattaaacaagactgctgtattttaatttcagaaattgaaaagggataggaggaaggggaaaatgctgggctggtgtgaagcgatagcccgtaaccctcacagaattccaaacaacacgcgaacacccgagatctcaggggatttggctgacgcctcacaaacctccacattgaatgaaaaatccccagggcgatctgcaagtcgatcaagtaacatttcaaaagcaagcagcccaacaacagggacagctcccaggagccagtcaaggttgtctgtctgtccatccactcaggacatctgcagaatctgtcactgcgaaggggatgaagagagccccctcatcacaccctgtcgctgcactgggacactgcgctttgtccaccagtcctgcctccaccagtggataaagagctcagatacacgctgctgtgagctctgcaagtatgacttcataatggagaccaagctcaaacccctccggaagtgggagaaactacagatgaccacaagtgaaaggaggaaaatattctgctctgtcacattccacgtaatcgcgatcacctgtgtggtttggtctttgtatgtattgatagaccggacagcggaggaaatcaagcaaggcaatgacaatggtgtccttgaatggccattttggacaaaactggttgtggtagccattggcttcacaggaggtcttgtcttcatgtacgtacagtgtaaagtctatgttcagttgtggcgcaggctgaaggcctacaaccgtgtgatctttgtacaaaattgcccagacactgccaaaaaactggagaagaacttctcatgtaatgtaaacacagacatcaaagatgctgtggtagtgcctgtaccacaaacaggtgcaaattcactgccatctgcagagggtggcccccctgaagttgtatcagtctgatggaacctgttgggagtttcttcaccgaagaatatctttctagccctcagccactacaaatgacagaagtgaccttgaattatttactcccttcagctcctcctttctcctactgacacatttttcctgactttgttcaaagaggaaaggagaaaaacaaacaaacagaccaaatgcccaggagcccatgaagtaatagcgtaaagtaaagtatgatatggaaatgtgaagtttgcaagagaatgatttccaagacaattaagaactactggggcaatgaatgcttttaggcagtaatcaaagattaaatggacccatgatactcttcttcacagtaacaggggaaaagttcaagaatacagacttgaattgcgatgtgtattacttctagggccttgtaatgttaactgtctcatctggaaataataactaacatatttggttttaagcctgaaattgtctgcattatccctaagtcacattggaagtgaacttggaggatgcatattttgatatgctttgacagctaacagatttgtatggtttagtggagtctggttattttgacagatgcatgttttttttaaatagatgcaatatacatttgaagacattgatatttggaattaattatgtttgtttaagtcacgcaaaagattttcagaaaatgttcggatataattagctctgttaaatacccacagaactgttatcaggtcttatatttattttcatctggttcctctaatacagtgctgtccaatagaaacacaacagccacaaatgcaggccacagatgcaaatatttaacttcccagtagccctattttaaaaagtaaaaataaatgtttgtttgttaaaaaaaaaa

SEQ ID NO: 39 - protein, 289 aa, Homo sapiens
MLGWCEAIARNPHRIPNNTRTPEISGDLADASQTSTLNEKSPGRSASRSSNISKASSPTTGTAPRSQSRLSVCPSTQDICRICHCEGDEESPLITPCRCTGTLRFVHQSCLHQWIKSSDTRCCELCKYDFIMETKLKPLRKWEKLQMTTSERRKIFCSVTFHVIAITCVVWSLYVLIDRTAEEIKQGNDNGVLEWPFWTKLVVVAIGFTGGLVFMYVQCKVYVQLWRRLKAYNRVIFVQNCPDTAKKLEKNFSCNVNTDIKDAVVVPVPQTGANSLPSAEGGPPEVVSV

SEQ ID NO: 40 - DNA, 1614 bp, Homo sapiens
  CDS (352)..(1092)
gcggccgcccggcccggccgcggatcagagactggagactgggtcttgctctgttacccaggctggagtgcagtggtgtgatcatagctcactacagccttgaactcctgggctcaagaggttgaggctccctcctcagcctcccaagtagctgggactacaggctttcagcaccatgcctggctaattcaaaaaaaccttcagagagatagggtctctctatgttgccctagctcgtctcaaactcctggcctcaagtgatcctcctgcttggacctcccaaagcgctgggattacaggctcctggaaccatgggcctcaggccctgaggatacggggctcccggtggccatgacgacgggtgactgctgccacctccccggctccctgtgtgactgctccggcagccctgccttctccaaggtcgtggaggctacgggcctcggaccgccccagtatgtggcacaggtgacttcaagggatggccggctcctctccaccgtcatccgtgccttggacacaccgagtgatggtcctttctgccggatctgccatgagggagcgaacggggagtgcttgctgtccccgtgtggctgcaccggcacgctgggtgccgtgcataagagctgtctggagaagtggctttcctcatctaacaccagctactgcgagctgtgccacacggagtttgcagtggagaaacggcctcgacccctcacagagtggctgaaggacccggggccgcggacggagaagcggacactgtgctgcgacatggtgtgtttcctgttcatcacaccgctggccgccatctcaggctggttgtgcctgcgcggggcccaggaccacctccggctccacagccagctggaggccgtgggtctcattgccctcaccatcgccctcttcaccatctatgtcctctggacgctggtctccttccgctaccactgccagctgtactccgagtggagaaagaccaaccagaaagttcgcctgaagatccgggaggcggacagccccgagggcccccagcattctccactggcagctggactcctgaagaaggtggcagaggagacaccagtatgaatgctgggctctccggaccctgcagcagagaggccagaggtagctggtgataccctgtcctgtggaaggacttccacttcaacacttccacttcaacagttcccgcacggcctgaacgcttcttaggccaagagacaccatgcggagcctagtctgtgatcctgtgtgaagatattttcagggtttttttttttttttttttgcatatggaggacaggtggacatggtcctgagctctggacggagcaggcaccctgatctcattctgaggtccacatggcaccttctgggccagcagctgtggccggtgtatcaagggcgcccttaaagctggaacattccagcaagcttcttgcgcttctctgcacccggcaggcccactttcctggcaccctcgactttatataaaagttgcactgcgtttcaaaaacccaccctgaatgaataaaaggagccctggctggacgtaaaaaaaaaaaaaaaaaaaaaaaaaaaaaaaaaaaaaaaaaaaaaaaaaaaaaa

SEQ ID NO: 41 - protein, 246 aa, Homo sapiens
MTTGDCCHLPGSLCDCSGSPAFSKVVEATGLGPPQYVAQVTSRDGRLLSTVIRALDTPSDGPFCRICHEGANGECLLSPCGCTGTLGAVHKSCLEKWLSSSNTSYCELCHTEFAVEKRPRPLTEWLKDPGPRTEKRTLCCDMVCFLFITPLAAISGWLCLRGAQDHLRLHSQLEAVGLIALTIALFTIYVLWTLVSFRYHCQLYSEWRKTNQKVRLKIREADSPEGPQHSPLAAGLLKKVAEETPV

SEQ ID NO: 42 - DNA, 1571 bp, Homo sapiens
  CDS (11)..(319)
accagaagagatggagctggacagagctgtgggggtcctgggcgctgccaccctgctgctctctttcctgggcatggcctgggctctccaggcggcagacacctgtccagaggtgaagatggtgggcctggagggctctgacaagctcaccattctccgaggctgtccggggctgcctggggcccctgggcccaagggagaggcaggcaccaatggaaagagaggagaacgtggcccccctggacctcctgggaaggcaggaccacctgggcccaacggagcacctggggagccccagccgtgcctgacaggtgactgaccacccccacactcctcccacggcttgtggctgcccttggctggaagtccagggtcatacgacgccattgccagaatgaagtgacaaatatgaacagaagaaaatggttgcttgcccgtttccttgtcccctaattattcaaggaagtgacaaatattgtccagacaacttacatacactccatggcaatttttttctatttataatctacgccagaaggccgggtgcccaggagctgaaggtgggggtgacactgggaggtgggagggccaggccaactggctgcggccatcacagctgcgtggccctgggggccgtgtccctgtccaggcctcagagtcccgctctgttcatacagacgcctatggccctgcttcttcctcccaggcctccctcctactgcctgtgccctgcccagggctcctgtcctgcagccattccccgggttcccttcccaggcccgcgtacctgcaaggacctgctagaccgagggcacttcctgagcggctggcacaccatctacctgcccgactgccggcccctgactgtgctctgtgacatggacacggacggagggggctggaccgtgagtgtggggctgggcagaggcggtcagcctgggagtcccggaggccaggctgcacacctggtgggagaacacactctggaattctctattctcctggtcggggacagtcagagatgatgggggagatgaccggtgggtaaaagtccaggcctttgggggaggctgggagaggttgggtgctctcctgctgtgtggcttggggcttgttgcctcccctctctgagcccccatttcctccttcatcctgtggagtctgtgaggagaacaggtgggcgtgctggtgaccccgcctgtgatgctctgccaactacaaatgctgctcctctggagggcgggtcccccgtgctgtgggacgtcggcctggcccccccggctcctgtcccctggcttctccacaggttttccagcggagggtggatggctctgtggacttctaccgggactgggccacgtacaagcagggcttcggcagtcggctgggggagttctggctggggaatgacaacatccacgccctgaccgcccagggaaccagcgagctccgtgtagacctggtggactttgaggacaactaccagtttgctaagtacagatcattcaaggtggccgacgaggcggagaagtacaatctggtcctgggggccttcgtggagggcagtgcgg

SEQ ID NO: 43 - protein, 102 aa, Homo sapiens [sequence omitted]

SEQ ID NO: 44 - DNA, 4450 bp, Homo sapiens
  CDS (1642)..(3000)
cccacctcggccacactgcttcgctcccccctccccaccttcagctggcaccgcaaacaagccttacctcgttgcatctgcgaggagaggtagcaacagcgagcctagccagccagaggcggtggagaggaggaaggggcggggtgggggggcagagagcgggccgaggccgcccttggtgggggtagcgggggcagagctgccgagcagacccggcagccgccctcctccgcccccaccctccagaagcactcttgcctgagaaccgaattattccactagtattatttcattttttattactccccctcctcactccccagccgcccccacccccacgccggcctcgcctctggttgcatggcagcgctgcccgggcgcgggggctcagggctggcccccagggaagggggaggaggaggaggatcatgaaggccggagtcgcgaccgcgccggacggcgggcagcagccagaggacgagccggagcagcccccgccccggagacacccggacgccgagcagcagtcgcctccgccgccgcagcagcagctgcgggcgcgggccgacccggagccggaggaggagggcgaccgcgacccagaggaggaagaggaggagaaggcgaggccactcgcccgcccggacctgcccttctctcggctcttgcccctcgaccgaagggacctttgatggaaccgaggggagggcgccacggatttgccgactgcagcaggggtgggctgggggctgagataatgtaaccactcctttctcctgttctctcccacacgcccctctcctctacccctattctctgctccactgccctctcaccccggtacacacacccttcctctagccaggatcttcatgctcaggaaggaggcgcctctgcaagggttaaacgatcttttctttttctcccatcctttttccttcccaaacctctattttacctcccttctcctaattggctttcccctcttcggtgtaaccctttggctgcggaggcaaagcacaagcccctagcccagtttcacctgcaacacccctcccccacccaactgctctcttaaaagcaactctggtgcttctgggggttaattgccccagttttctgcccaggagaattaaaacttctcccaatcttctctcctcccctaccttgactcccccaacccctaccacctgagaaaaacgatcttttctctctcacacatgcagtcctcaattcttcattgagctagttttctctaagcccagctcaatccactccagatttgatttacaattgtccccacccttttatataaaagaaagatttctcactgcgtaggaatttgagaagaacccaataatcctttcctggggaacttttaaacaattcgacattgatttaaacaattcgacagaggctctagtggcccctccaccacgcttcttcaatccctctgctcctaccagtgtcctcaggtcaaaagcagaaaggagacacactgaacaaagttggaggttggggtgggtgtgtgagggcaagaaaaacttttttgttattgggctttccaggtggagttcagaaccagtgactcacacttctcagtcctgggagcaatttatttgctacttggaggggttgtaagaaaagccagtgagaaagcagactccccccacaacacagatccactgtggacccccaaaacctgtcctgtccccctcttttaagactccagccacccctcttgggctctctacttccacggggcacatgctgatgcccctgtgtgggctgctctggtggtggtggtgctgctgctccggctggtactgctatggattgtgtgccccagccccccagatgttgcgccaccagggtctcctcaagtgccgctgccgcatgctcttcaatgacctgaaggttttcttactgcggcgccctcctcaagcgcccctgcccatgcacggcgacccccagccccccggtttggcggccaacaacacccttccggctctgggcgccggggggtgggcaggctggaggggcccccgagaagtggtgggcagggagccccctcctgtgccacctccaccccccttgccaccttcttctgtggaagatgactggggtggcccagccacagagccacctgcctcgctgctcagcagtgcctcctcagatgacttctgtaaggagaagaccgaggatcgctactcactgggcagcagcttggacagtggtatgaggaccccactctgccgcatctgcttccaggggccagaacagggggagctgctgagcccatgccgctgtgatggctcggtcaagtgcacacaccagccttgcctcatcaagtggatcagcgagcggggctgctggagctgcgagctgtgctactacaagtaccacgtcatcgccataagcacaaaaaatcctctgcagtggcaggccatctctctgacggtcattgagaaggttcaggttgcagccgccatcctgggctccctcttcctcatcgccagtatttcttggctcatctggtcaactttcagcccctcggcaagatggcagcgccaagaccttctcttccagatctgctacgggatgtatggcttcatggacgtggtgtgcataggtctcatcatccatgaaggaccctcggtgtaccgcatctttaaacggtggcaggctgtcaaccagcagtggaaagtgctgaactatgacaagacaaaagacctggaggatcaaaaggcaggaggcaggaccaacccccggacctcctcatccacccaggccaatatcccctcctcggaagaggagaccgcaggcacccctgcccctgagcagggccctgcccaggctgccggccacccctcaggccctctgtcccatcaccactgtgcttataccatcctgcacatcctgagtcacttgagacctcatgaacagcgaagtcccccaggcagcagccgagagctggtcatgagagtcacgacagtgtgagagcagaggcccggaaggaaggccatgaccaccactgagggcccggagcagggtggggaggtgcagtggcacccccggagccaacagagggagcaggcagagggtgggggacctggcgggagccctggggtagtgtcagagcgggagtgaggctggtgcaggagcagttctgctatttccaatcagtcaatgccactctccacaacaacaatgaaaaccaacaccaactcaacaacaaagtgcaatacaggctgaacctggcccaacagaaaaaccctgccccaatgcacctgcaggcaaggtacccgaagaagcagaggctgagggcaggcaaagcctgtgtgactgtggcagtgccggaggccaagggggccaagaggaaaagcatctgtggtctgccctgctctcaccctgtttggttttgtttctcctggggctgtgttctgcaggcagccagaaaaggaggaggcacgggtgagctggcagggacacactgcctttggggctcctgggctcatttggatgagcaagattcgctgacaaatggctgtggggatggtggggtggatggtcagggagggatcctcagggagggatatgctggtgtgagcagccagagggagagtgtgtctccttcctgaaggaacttccaaatggaactcccgatttcaggtgggctaaaagagggcttaggtttggaaaagggtgtccttctgtgcccttgttaatttattttatagtgatttggttcaaagatgtttacaggacacacacacacacacacacacacacacacacacacacacacccctagagaaaagtacagatttccagtggatatttcaagcacagttctgctgctgtggcttcagctttggaagctgtcaatcccggagcaactttcccaactacccaaccccaccatggccaggacatgtgcaatgccagccccttcttgtcttggcacatgcacagacccagtcccctcacggtagggcacccctgacctacgggcttccaagagagcagctgcagtggttgggaggagcttgaccagtgtgccccaaggagtggagtagagcccaatctaagtattccttgctgcttggaaccctccctgtttggaaccctccccaaagaggcagtcaggctgatgctcagtgctttgtgctccctgctccttcccgcgtagccaggtgggcccaagggtgcctggcagggagcactacccctggacccctcctgctcgctctggggaccctgccagggaaggccactgggtgttcacctgcaaagtttctggttgtcactgcacagtggtcgcgtcatccatgggtattaaaaggacactgtcaagtacttttttaaactagtttttagggttttttaaaactctctgttgtttgtaatattctcttaaaagcttgaaaataaaacttctttccctacc

SEQ ID NO: 45 - protein, 452 aa, Homo sapiens
EKPVRKQTPPTTQIHCGPPKPVLSPSFKTPATPLGLSTSTGHMLMPLCGLLWWWWCCCSGWYCYGLCAPAPQMLRHQGLLKCRCRMLFNDLKVFLLRRPPQAPLPMHGDPQPPGLAANNTLPALGAGGWAGWRGPREVVGREPPPVPPPPPLPPSSVEDDWGGPATEPPASLLSSASSDDFCKEKTEDRYSLGSSLDSGMRTPLCRICFQGPEQGELLSPCRCDGSVKCTHQPCLIKWISERGCWSCELCYYKYHVIAISTKNPLQWQAISLTVIEKVQVAAAILGSLFLIASISWLIWSTFSPSARWQRQDLLFQICYGMYGFMDVVCIGLIIHEGPSVYRIFKRWQAVNQQWKVLNYDKTKDLEDQKAGGRTNPRTSSSTQANIPSSEEETAGTPAPEQGPAQAAGHPSGPLSHHHCAYTILHILSHLRPHEQRSPPGSSRELVMRVTTV

SEQ ID NO: 46 - DNA, 3896 bp, Homo sapiens
  CDS (293)..(1129)
ggcgcccgcggtccatggaccggaacctcgggccgacggacgggaacccgggccgcgatcgccgcctccccgcctcaggctcctcctcctcgctctccgccgcctccgccggactcccgcaggccctgcaccgccgccgccaggctagcggagctgccccgggaagctgggtgacgggttcgcggctgccgccggactgcggcctactccgccgcctctcagtgctattgtccctgggcctggccttgagcgggtccactggggaaggccgtgtgcgccggctccgcggaagatgccggaccaagccctacagcagatgctggacagaagttgctgggtttgttttgctactgatgaagatgatagaacagctgaatgggtgagaccatgcaggtgcagaggatctacaaaatgggttcaccaggcctgtctacaacgctgggtggatgaaaagcaaagaggaaacagtacagccagagtggcatgtcctcagtgcaatgctgaatacctaatagtttttccaaaattgggtccagtggtttacgtcttggatcttgcagatagactgatctcaaaagcctgtccatttgctgcagcaggaataatggtcggctctatctattggacagctgtgacttatggagcagtgacagtgatgcaggttgtaggtcataaagaaggtctggatgttatggagagagctgatcctttattccttttaattggacttcctactattcctgtcatgctgatattaggcaagatgattcgctgggaggactatgtgcttagactgtggcgcaaatactcgaataaactacaaattttaaatagtatatttccagggataggttgtcctgttcctcgaattccagctgaggccaatcctttagcagatcatgtctctgctactcgaatcttgtgtggagcccttgtctttcctactattgctacaatagttggtaaattgatgttcagtagtgttaactctaatttacaaaggacaatcttgggtggaattgcgtttgttgccataaaaggagcatttaaagtttacttcaaacagcagcaatatttacgacaggcacaccgcaaaattctgaattatccagaacaagaagaagcataaaactgacttctggttgttctgcagttctctcatccttatgaatctgttgtgttgttttgattccatcattaatgcacttgtggagacttgtgataagctgctgctcctatattttttaagaaatataataaagcacttagggcaggggaaatcatctcggtaatcatggaacctaaggatgtgatttgttttcattgtttgtatgtactacttttatggcagtcatatgaaccattatcttagcatggtaaacctgggttttgttcatattttctccagacagaaatgcaaagatcaaactgtgcaaatattaaaaaaatgcacatgctgttttattcaaatgcctcttttgtacatgttcatgtttagtgttttctcagaatcagcaactcaaggtactatgaggatttttctcactgacataatttgattacatactaaataagaggatatgttaatatgaggaaatgtaaattaaattagttataaataaataaccaaaaatgtatgtaaacattcaaatgattatctgaacaaatgagattttgtggtgttttctttaacccatgtgatgtcctccaaaatgtgtagggtaaaaattcacagggcttccagatcactttttcaatattaaattttatttacataatgttgacatctcatacttcatgaagtaattttgactcatgcagtcgtgtgtgtgtgtgtgtgtgtgtgtgtgtatttgtgtgtttcagtgtttcatcaggtccttccatctctgggagttttttcaacccatatttctagaaattacagctgccagtttatagtagtttgagcagaggatgatttgcaaaaataaaaataaagttattttactctcctcttgcattgattcagttattgagatattcgttgatcacctcctatttcccaggcactgtgcagagtgctgtaggacatagtggtgaacaagacagacttggtccaagctcttacagagttgacagtctagtttcagcacatagaaaaccagcagtgaataaaccaaggtgaggccttgagctctttgtttttatattaatgaggagaaaagtatgaaaacaagaagtcttaagtaaatattgaggtcattgtttttggcttaagtttattatagaaaatcaacactaatgtttttagattttaattgtcctattgatgaggctagcaccttaatcactgtttagttttgtattcatttttaaaagcaattattgaagccattttcaatagattggccattttaatgttcagcaacctgaatggttatttttgttaattaaaattaaatttttaagagatattttcaaaaccctatttattttcttgttcacagtaatgcatgtcaataataaatgtttccccttactgataagcggccactttaggagtgtagcaaatatagattgagctatgttagtttgcaataatatatgttaactttagtaattaaagactggtttctatagtatgaatgtcttaattttgagttatatgatgtttatttgaatgactgttgaacattcaaatttgtattatttggagatgaagatttgactaacagtgagccttattaagaacactactacagttctgaaggggaaatataacatctatggttatatattttaaaaacttagattataggctgtaattataaaatatattggcttttgttttcaatgtgaaagatacattaaatggacacatatcttgcaaaattttgttgtatagaacagtttttaggcagcctttactaaagttatgcaaacacacagttccccatttactacattaatgcccttgacagggagtagctgcttggttttataggtattagggctcatgaaggccggggaacaactctaatccttttggggcaggagggagggtttttttagggttgcggggagggaactgcaagtgcctgaggaccagagtggcctctagccccgaattgaacacttttaaacctaaagagccttattattattagctcgagaaataccacatgccagtcttcccaggaaggtgacctgcctgaccatgaagcaacaaaagagcataggcagacttaaagttggatagacctgggttcaaatcatagttcagctttcaataactgtatgtatgattttggctaaaggatttgactttctctcaactgcagttttcttaatctttgaaatgcaaataatgtttatctcagaagactatttctaggattaactgagagattaaaggaacagcagcacttaggctcaaaatcgcccctacaaagcagtagttgtttcttaattgctaagtaatctgtttgcactctaggaaaagtaaatggagaggtaaaagaaaggcattggggacaaatgaaagagatgatgtagatttttcggatgggtgggtatattttattttccagtttattttcatatttctttactgtgttatttctggaaactttaatgttttaaacctcttttataaaattcaatgacgactactagaattctattcttttcaaaagttaacttatctccaaagtggctgaaaatattgtttaccattacatgattatgaaatgactgtgaagaatataaaattaaacttagtgacttaattagtcactgccttgctaactttgctgtaatttttttaaacgtcttgtttttaatatagcaaactatctcaatactggctggattaggttaaataaagaatttttatgttcaaaaaaaaaaaaaaa

SEQ ID NO: 47 - protein, 278 aa, Homo sapiens
MPDQALQQMLDRSCWVCFATDEDDRTAEWVRPCRCRGSTKWVHQACLQRWVDEKQRGNSTARVACPQCNAEYLIVFPKLGPVVYVLDLADRLISKACPFAAAGIMVGSIYWTAVTYGAVTVMQVVGHKEGLDVMERADPLFLLIGLPTIPVMLILGKMIRWEDYVLRLWRKYSNKLQILNSIFPGIGCPVPRIPAEANPLADHVSATRILCGALVFPTIATIVGKLMFSSVNSNLQRTILGGIAFVAIKGAFKVYFKQQQYLRQAHRKILNYPEQEEA

SEQ ID NO: 48 - DNA, 4611 bp, Homo sapiens
  CDS (1)..(2916)
gtgtcgctcgctttctgtcagcctctctccctctccctctcccctctccttcctctcgcttcctctctcgcacctgagcgtacgcacctgcccgggcccggctccctcctcctctcccctccctctttccccgcccggccgcgggagcctcgtggctgcgtcaccgccgcccccccagacaagatggacaccgcggaggaagacatatgtagagtgtgtcggtcagaaggaacacctgagaaaccgctttatcatccttgtgtatgtactggcagtattaagtttatccatcaagaatgcttagttcaatggctgaaacacagtcgaaaagaatactgtgaattatgcaagcacagatttgcttttacaccaatttattctccagatatgccttcacggcttccaattcaagacatatttgctggactggttacaagtattggcactgcaatacgatattggtttcattatacacttgtggcctttgcatggttgggagttgttcctcttacagcatgccgcatctacaagtgcttgtttactggctccgtgagctcactactgacgctgccattagatatgctgtcaacggaaaatttgttggcagattgtttgcagggttgttttgtggtgacgtgcacactgtgtgcattcatcagcctggtgtggttgagagagcagatagtccatgggggagcaccaatttggttggagcatgctgccccaccgttcaatgctgcggggcatcaccaaaatgaggctccagcaggaggaaatggtgcagaaaatgttgctgctgatcagcctgctaacccaccagctgagaacgcagtggtgggggaaaaccctgatgcccaggatgaccaggcagaagaggaggaggaggacaatgaggaggaagatgacgctggtgtggaggatgcggcagatgctaataacggagcccaggatgacatgaattggaatgctttagaatgggaccgagctgctgaagagcttacatgggaaagaatgctaggacttgatggatcactagtttttctggaacatgtcttctgggtggtatctttaaatacactgttcattcttgtttttgcattttgcccttaccatattggtcatttctcccttgttggtttgggatttgaagaacacgtccaagcatctcattttgaaggcctaatcacaaccatagttgggtatatacttttagcaataacactgataatttgtcatggcttggcaactcttgtgaaatttcatagatctcgtcgcttactgggagtctgctatattgttgttaaggtctctttgttagtggtggtagaaattggagtattccctctcatttgtggttggtggctggatatctgttccttggaaatgtttgatgctactctgaaagatcgagaactgagctttcagtcggctccaggtactaccatgtttctgcattggctagtgggaatggtatatgtcttctactttgcctccttcattctactactgagagaggtacttcgacctggtgtcctgtggtttctaaggaatttgaatgatccagatttcaatccagtacaggaaatgatccatttgccaatatataggcatctccgaagatttattttgtcagtgattgtctttggctccattgtcctcctgatgctttggcttcctatacgtataattaagagtgtgctgcctaattttcttccatacaatgtcatgctctacagtgatgctccagtgagtgaactgtccctcgagctgcttctgcttcaggttgtcttgccagcattactcgaacagggacacacgaggcagtggctgaaggggctggtgcgagcgtggactgtgaccgccggatacttgctggatcttcattcttatttattgggagaccaggaagaaaatgaaaacagtgcaaatcaacaagttaacaataatcagcatgctcgaaataacaacgctattcctgtggtgggagaaggccttcatgcagcccaccaagccatactccagcagggagggcctgttggctttcagccttaccgccgacctttaaattttccactcaggatatttctgttgattgtcttcatgtgtataacattactgattgccagcctcatctgccttactttaccagtatttgctggccgttggttaatgtcgttttggacggggactgccaaaatccatgagctctacacagctgcttgtggtctctatgtttgctggctaaccataagggctgtgacggtgatggtggcatggatgcctcagggacgcagagtgatcttccagaaggttaaagagtggtctctcatgatcatgaagactttgatagttgcggtgctgttggctggagttgtccctctccttctggggctcctgtttgagctggtcattgtggctcccctgagggttcccttggatcagactcctcttttttatccatggcaggactgggcacttggagtcctgcatgccaaaatcattgcagctataacattgatgggtcctcagtggtggttgaaaactgtaattgaacaggtttacgcaaatggcatccggaacattgaccttcactatattgttcgtaaactggcagctcccgtgatctctgtgctgttgctttccctgtgtgtaccttatgtcatagcttctggtgttgttcctttactaggtgttactgcggaaatgcaaaacttagtccatcggcggatttatccatttttactgatggtcgtggtattgatggcaattttgtccttccaagtccgccagtttaagcgcctttatgaacatattaaaaatgacaagtaccttgtgggtcaacgactcgtgaactacgaacggaaatctggcaaacaaggctcatctccaccacctccacagtcatcccaagaataaagtagttgtctcaacaacttgaccttcccctttacatgtccttttttgtggacttctctctttggagatttttcccagtgatctctcagcgttgtttttaagttaaatgtatttgacttgtgttctcagcattcagagagcagcggtgtaagattctgctgttctccctggatcttctgacattactgctgtctgagatttgtatatgtgtaaatacaagttccttgataccctaaaaccttggattaaacagaatgtgcattgtacatctttaaacaaaatgtatattaatttattaaatctagttgtcactttattttggacctgctgtgatctcgacaggaaacgtgccacagagcagtagtgcgcaggcaagacttttcagtgacgccttgtggaacgcagttcatgatgtcctagcagctctcactaagggaactgtacattctttctttcttggctattcagaccttaccaagaacgttaaaggaaacaagtagaaatcagcagtggagtgtctgtggtaagaaaacatgaactttatgcttcactgttagttgtttgtggaagttattttgtataacaccaaagctgttgtacatttcctactgcctgatttttttcatgtgtctgtgtttgtaatattgtatagtatcttgtgctaggtgaggaaattatttttaattttgataatttaatattcctagtgtgatcagcattgggagttgggtttcagtggggcatgtctatacttagagaaaaaaagtccaaatgaagattttcatgagtcagcccccccgcccgcccccaccccacacccacatcctctcttttccacacacaactatctgtttattttttgtagcagtggccgaaagtcctgcaaggtcataaatctttcagagtgacatcaccaactgtactgcatcttactggatttaggacttctgagatgcttgtgaagtatagatgtggttgtggtcttagattgacagcattagagaagactggttagaacatctggtctcgctggttagtgcctcgttggctgaggactaggtgtgcatttctcctagcttttcatcaggaaatcccaaagtttccaaagctttttgtttacagaataaaacttcaaataaaaccaattcattatttgtccagaaggaagcttggctgagctggccttttaacataggaatgtatttcgttggaaacattctgaaaaatctcagagaactgaacccttacaaactttgttttccctcataaccaaagcttcaggttagaagtttagaaaaatagaatggttgggtacatgatctaaatgtttaatgctaaaggtatatcgtaagggtagtgtttgtttttgaacgataatttagaagttctcatagaaagcgtataacataggtcttcagaaactataaaagaattttcatatagtattaaaatccatagactaaaatctgagaattttttaacatatgcaagtcagccaaacataagctaccaaaataaagagcaatgtgttctggctgttttatacttcaacaattttttccctaagtggtaagcaattactttaaaacatatttttaaaaacatcggtatcgggagctgcggtggctccggccggttgtcctggcacacaaggaggcgaggctatgcgttcgaggccaacctaggcaaaattgg

SEQ ID NO: 49 - protein, 971 aa, Homo sapiens
VSLAFCQPLSLSLSPLLPLASSLAPERTHLPGPGSLLLSPPSFPARPREPRGCVTAAPPDKMDTAEEDICRVCRSEGTPEKPLYHPCVCTGSIKFIHQECLVQWLKHSRKEYCELCKHRFAFTPIYSPDMPSRLPIQDIFAGLVTSIGTAIRYWFHYTLVAFAWLGVVPLTACRIYKCLFTGSVSSLLTLPLDMLSTENLLADCLQGCFVVTCTLCAFISLVWLREQIVHGGAPIWLEHAAPPFNAAGHHQNEAPAGGNGAENVAADQPANPPAENAVVGENPDAQDDQAEEEEEDNEEEDDAGVEDAADANNGAQDDMNWNALEWDRAAEELTWERMLGLDGSLVFLEHVFWVVSLNTLFILVFAFCPYHIGHFSLVGLGFEEHVQASHFEGLITTIVGYILLAITLIICHGLATLVKFHRSRRLLGVCYIVVKVSLLVVVEIGVFPLICGWWLDICSLEMFDATLKDRELSFQSAPGTTMFLHWLVGMVYVFYFASFILLLREVLRPGVLWFLRNLNDPDFNPVQEMIHLPIYRHLRRFILSVIVFGSIVLLMLWLPIRIIKSVLPNFLPYNVMLYSDAPVSELSLELLLLQVVLPALLEQGHTRQWLKGLVRAWTVTAGYLLDLHSYLLGDQEENENSANQQVNNNQHARNNNAIPVVGEGLHAAHQAILQQGGPVGFQPYRRPLNFPLRIFLLIVFMCITLLIASLICLTLPVFAGRWLMSFWTGTAKIHELYTAACGLYVCWLTIRAVTVMVAWMPQGRRVIFQKVKEWSLMIMKTLIVAVLLAGVVPLLLGLLFELVIVAPLRVPLDQTPLFYPWQDWALGVLHAKIIAAITLMGPQWWLKTVIEQVYANGIRNIDLHYIVRKLAAPVISVLLLSLCVPYVIASGVVPLLGVTAEMQNLVHRRIYPFLLMVVVLMAILSFQVRQFKRLYEHIKNDKYLVGQRLVNYERKSGKQGSSPPPPQSSQE

SEQ ID NO: 50 - DNA, 1925 bp, Homo sapiens
  CDS (1)..(678)
cctggttccttattccggtttgcagtccccccagcacttgggagtaatttgaccgacaatgtcatgatcacagtagatattattccttcaggttggaattcagctgatggtaaaagtgataaaactaaaagtgcgccttcaagagatccagaaagattgcagaaaataaaagagagcctccttttagaggactcagaagaagaagaaggtgacttatgtagaatttgtcaaatggcagctgcatcatcatctaatttgctgatagagccatgcaagtgcacaggaagtttgcagtatgtccaccaagactgtatgaaaaagtggttacaggccaaaattaactctggttcttcattagaagctgtaaccacctgtgaactatgtaaagagaagttggagcttaacctggaggattttgatattcatgaactacatagagctcatgcaaatgaacaagctgagtatgagtttatcagctctggtctctacctagtggtgttattgcacttgtgcgaacaaagcttttctgatatgatgggaaatacaaatgaaccaagcacacgtgtccgatttattaaccttgcaagaactcttcaggcacatatggaagatctcgaaacttcagaggatgattccgaagaagacggagaccataacaggacatttgatattgcctaacttcatataagacagatggatgatctgtgaacataagtgtttattaaaaatggcaattaaatataaattacttttgtgggggaatgcctaataaatacattgactatatataaaatgaatatatacatacacatgtatgcctgtatatatatattcattctccagtgttgctgaattaaaattctgctggactttttaacatagcaaatccgatgtttataaactggtaatcaaaaaggttttttcttttaggtgagtgggaaagtattacccttgttttaaatatctaagcaatgcctatcaacccttttttgtgttatgattactgtagtcatatttatgaaaaaaggtttgtgttttactcttgctagtgagaaaagtgggacaaaatatacttttgaaataaaatgctatatggcacctaattattttttcttttaaaatgccttaagttgcagtctcattttgataatcatttgcttccagtgtttaaaaattaaaaaaagaatggggagaaggttatgagaagagcattattaagtttccaaatttaatttgaattccaaattcacctagcaataaaatctaatttttaaaaagtatataaatataaaatgtataaatgatggatagatttttgtattgatttgcaaaatgcagattatatttgataggctatagtatgtagatattccttttaggaatattacagctgtaaattatatgagacttgccagtcaaatgctatttggtttaaaaaaattattgcaatctcaagttaatggaatatttttaaatcccacattcagagtttaaaacactggttttcaatgtgttttttagtgttgtcacttgtttatagataaatatataaataacctgtttggatcctggtcctttttaactgttccttggtaattctgagcatttatttgatgacttaatatttttcactacctttggagaacagatgaacattattcaccatgaatggatctatactgtgtggtcatgagttgtgtatacttccataacactgtatttttcttctgtcagtacccttaggatacactttaaaacaccttaaggtctgatgttatggcaacaaactactttttcaaacctaaataggaaccatgtaatttctcaaaagtgattgaacagtttgcccacacttagtttgttggtcttatgtaaaacattggctcaaaataaagtacacactgatttaaaaaaaaaaaaaaaaa

SEQ ID NO: 51 - protein, 225 aa, Homo sapiens
PGSLFRFAVPPALGSNLTDNVMITVDIIPSGWNSADGKSDKTKSAPSRDPERLQKIKESLLLEDSEEEEGDLCRICQMAAASSSNLLIEPCKCTGSLQYVHQDCMKKWLQAKINSGSSLEAVTTCELCKEKLELNLEDFDIHELHRAHANEQAEYEFISSGLYLVVLLHLCEQSFSDMMGNTNEPSTRVRFINLARTLQAHMEDLETSEDDSEEDGDHNRTFDIA

SEQ ID NO: 52 - DNA, 1863 bp, Homo sapiens
  CDS (245)..(1120)
agcggatggtaggtatagggctgtggatatcgtcagtttgaccaagtattccaagaggccccatggagtttgtcatctgtaatagaggacgttagtgcattagtgatgaaaaaactgcttaccactgactattgtgatctcccagggagtataaggcagctccgcacttgaaatccatggcccaaaatgactctaccagtggagactctcttctgtgaagaagacgaccagataagaggttgggatgagcatgccactgcatcagatctctgccattccatcccaggatgccatctctgctagagtctacagaagtaagaccaaagaaaaggagagggaagaacagaatgagaagactttgggacatttcatgagtcattcaagcaacatttctaaggctgggagtcctccgtcagcatcagctccggctccggtgtcctccttctctcgcacttctatcacgccatccagccaggacatctgcaggatctgccactgtgaaggagatgatgagagccccctgatcaccccctgccactgcacaggaagcctccacttcgtgcaccaggcctgcctgcagcagtggatcaagagctccgacacgcgctgctgcgagctctgcaagtatgagttcatcatggagaccaagctgaagccactgagaaaatgggagaagttgcagatgacgtccagcgagcgcaggaagatcatgtgctcagtgacattccacgtcattgccatcacatgtgtggtctggtccttgtatgtgctcattgaccgtactgctgaggagatcaagcaggggcaggcaacaggaatcctagaatggcccttttggactaaattggtggttgtggccatcggcttcaccggaggacttctttttatgtatgttcagtgtaaagtgtatgtgcaattgtggaagagactcaaggcctataatagagtgatctatgttcaaaactgtccagaaacaagcaaaaagaatatttttgaaaaatctccactaacagagcccaactttgaaaataaacatggacatggaatctgtcattccgacacaaactcttcttgttgcacagagcctgaagacactggagcagaaatcattcacgtctgattgtgtgcgggttgtcattttcctggacatccatgaagagctgaaggaaattgtttactgccaattgtatacctttcttatgtcctttaatagcatagactggacaggtgactatttatagtggcttctctttttctaaaccctccttagtctcctagaaaaccttcctgtgggccaggcatgcctgggtcctgcctctgcctggcagctctgtgggaaagtggaagaccccatgatgacatcatggggagccagcagagttcctgcccatggtcttgagctgaatgagagaataaaatgccaatcccaagggaagaggaggagcaggggtgcccaggccctgatacccagccgcctccagcttgcagtggtccccagcctggagcagagcattggggagtgtctagccatgacgagaagattccctctgcatcacggcgaaccccaggagatggtattgaaacagacccccaaacacagactcctgcctgccctctgccgatgctgcctcctccatgctcttgagcaggtggagccatggtgctctgtggtggcgcatgattcactgagcaaacagcactttacagaagaaaatctttattttgtaatatgtgtgtccagcgggattgacactcaaaaaaagtctcacttagaaatcttcccttccttacctttgtatctcctttacatcatgagagatcaaaaatccattttgccttacatatgcaaaaaaaaaaaaaaaa

SEQ ID NO: 53 - protein, 291 aa, Homo sapiens
MSMPLHQISAIPSQDAISARVYRSKTKEKEREEQNEKTLGHFMSHSSNISKAGSPPSASAPAPVSSFSRTSITPSSQDICRICHCEGDDESPLITPCHCTGSLHFVHQACLQQWIKSSDTRCCELCKYEFIMETKLKPLRKWEKLQMTSSERRKIMCSVTFHVIAITCVVWSLYVLIDRTAEEIKQGQATGILEWPFWTKLVVVAIGFTGGLLFMYVQCKVYVQLWKRLKAYNRVIYVQNCPETSKKNIFEKSPLTEPNFENKHGHGICHSDTNSSCCTEPEDTGAEIIHV

SEQ ID NO: 54 - protein, 272 aa, Homo sapiens
MTSSHVCCNFLNMWKKSKISTMYYLNQDAKLSNLFLQASSPTTGTAPRSQSRLSVCPSTQDICRICHCEGDEESPLITPCRCTGTLRFVHQSCLHQWIKSSDTRCCELCKYDFIMETKLKPLRKWEKLQMTTSERRKIFCSVTFHVIAITCVVWSLYVLIDRTAEEIKQGNDNGVLEWPFWTKLVVVAIGFTGGLVFMYVQCKVYVQLWRRLKAYNRVIFVQNCPDTAKKLEKNFSCNVNTDIKDAVVVPVPQTGANSLPSAEGGPPEVVSV

SEQ ID NO: 55 - protein, 25 aa, Homo sapiens
MEEPQSDPSVEPPLSQETFSDLWKL

SEQ ID NO: 56 - protein, 253 aa, Homo sapiens
MTTSRCSHLPEVLPDCTSSAAPVVKTVEDCGSLVNGQPQYVMQVSAKDGQLLSTVVRTLATQSPFNDRPMCRICHEGSSQEDLLSPCECTGTLGTIHRSCLEHWLSSSNTSYCELCHFRFAVERKPRPLVEWLRNPGPQHEKRTLFGDMVCFLFITPLATISGWLCLRGAVDHLHFSSRLEAVGLIALTVALFTIYLFWTLVSFRYHCRLYNEWRRTNQRVILLIPKSVNVPSNQPSLLGLHSVKRNSKETVV

SEQ ID NO: 57 - protein, 410 aa, Homo sapiens
MLMPLCGLLWWWWCCCSGWYCYGLCAPAPQMLRHQGLLKCRCRMLFNDLKVFLLRRPPQAPLPMHGDPQPPGLAANNTLPALGAGGWAGWRGPREVVGREPPPVPPPPPLPPSSVEDDWGGPATEPPASLLSSASSDDFCKEKTEDRYSLGSSLDSGMRTPLCRICFQGPEQGELLSPCRCDGSVKCTHQPCLIKWISERGCWSCELCYYKYHVIAISTKNPLQWQAISLTVIEKVQVAAAILGSLFLIASISWLIWSTFSPSARWQRQDLLFQICYGMYGFMDVVCIGLIIHEGPSVYRIFKRWQAVNQQWKVLNYDKTKDLEDQKAGGRTNPRTSSSTQANIPSSEEETAGTPAPEQGPAQAAGHPSGPLSHHHCAYTILHILSHLRPHEQRSPPGSSRELVMRVTTV

SEQ ID NO: 58 - protein, 278 aa, Homo sapiens; identical to SEQ ID NO: 47

SEQ ID NO: 59 - protein, 971 aa, Homo sapiens; identical to SEQ ID NO: 49

SEQ ID NO: 60 - protein, 225 aa, Homo sapiens; identical to SEQ ID NO: 51

SEQ ID NO: 61 - protein, 291 aa, Homo sapiens; identical to SEQ ID NO: 53

SEQ ID NO: 62 - DNA, 32 bp, artificial sequence; identical to SEQ ID NO: 8
  MARCH-II PHD domain reverse primer

SEQ ID NO: 63 - DNA, 34 bp, artificial sequence
  hUbc6 forward primer
atatgctagcgccatgaggagcaccagcagtaag

SEQ ID NO: 64 - DNA, 32 bp, artificial sequence
  hUbc6 reverse primer
atatgcatcctcactcctgcgcgatgctcctc

SEQ ID NO: 65 - DNA, 33 bp, artificial sequence
  hUbc7 forward primer
atatgctagcgccatggcggggaccgcgctcaa

SEQ ID NO: 66 - DNA, 32 bp, artificial sequence
  hUbc7 reverse primer
atagggatcctcacagtcccagagacttctgg